(19) United States
(12) Patent Application Publication
(10) Pub. No.: US 2005/0266032 A1
(43) Pub. Date: Dec. 1, 2005

(54) Dosage form containing multiple drugs

(75) Inventors: Viswanathan Srinivasan, The Woodlands, TX (US); Ralph Brown, Southlake, TX (US); David Brown, Colleyville, TX (US); Himanshu Patel, North Richland Hills, TX (US); Juan Carlos Menendez, Bedford, TX (US); Venkatesh Balasubramanian, Arlington, TX (US); Somphet Peter Suphasawud, Fort Worth, TX (US)

Correspondence Address: GREENBLUM & BERNSTEIN, P.L.C., 1950 ROLAND CLARKE PLACE, RESTON, VA, 20191, US

(73) Assignee: Sovereign Pharmaceuticals, Ltd., Forth Worth, TX (US)

(21) Appl. No.: 11/115,321

(22) Filed: Apr. 27, 2005

Related U.S. Application Data
(63) Continuation-in-part of application No. 10/736,902, filed on Dec. 17, 2003.
(63) Continuation-in-part of application No. 10/798,884, filed on Mar. 12, 2004.
(63) Continuation-in-part of application No. 10/910,806, filed on Aug. 4, 2004.
(63) Continuation-in-part of application No. 10/939,351, filed on Sep. 14, 2004.
(63) Continuation-in-part of application No. 11/012,264, filed on Dec. 16, 2004.

(51) Int. Cl. A61K 31/485; A61K 31/137

(52) U.S. Cl. 424/400; 514/282; 514/649

(57) ABSTRACT

A pharmaceutical dosage form comprising a first drug and a second drug, both of which are selected from decongestants, antitussives, expectorants, analgesics and antihistamines. The dosage form provides a plasma concentration within a therapeutic range of the second drug over a period which is coextensive with at least about 70% of a period over which the dosage form provides a plasma concentration within a therapeutic range of the first drug. This Abstract is neither intended to define the invention disclosed in this specification nor intended to limit the scope of the invention in any way.

### CROSS-REFERENCE TO RELATED APPLICATIONS

The present application is a continuation-in-part of U.S. patent application No. 10/736,902, filed Dec. 17, 2003, Ser. No. 10/798,884, filed Mar, 12, 2004, Ser. No. 10/910,806, filed Aug. 4, 2004, Ser. No. 10/939,351, filed Sep. 14, 2004, and Ser. No. 11/012,267, filed Dec. 16, 2004. The entire disclosures of these applications are expressly incorporated by reference herein.

### BACKGROUND OF THE INVENTION

1. Field of the Invention

The present invention relates to a pharmaceutical dosage form which comprises a first drug and a second drug, both of which are selected from decongestants, antitussives, expectorants, analgesics and antihistamines. The dosage form releases the first drug and the second drug so as to provide pharmaceutically effective plasma concentrations of these drugs over similar periods of time. The present invention also relates to a process for manufacturing the dosage form and to methods for alleviating conditions which can be alleviated by a combination of these drugs.

2. Discussion of Background Information

Conditions such as colds and allergies are accompanied by a variety of symptoms which frequently can not satisfactorily be ameliorated or treated with a single drug but require the administration of two or more drugs. Drugs which are indicated for the amelioration of cold and allergy related symptoms often belong to at least one of the following classes: antihistamines, decongestants, antitussives, expectorants and analgesics. To facilitate and simplify the administration of the different drugs that are required for the amelioration of the various symptoms that a patient is experiencing it would be expedient to administer all of these drugs in a single dosage form, or at least in as few dosage forms as possible. However, in many cases these different drugs have significantly different pharmacokinetic properties. For example, a single dose of a first indicated drug may provide a therapeutically effective plasma concentration for a period of time which is significantly longer than the therapeutically effective plasma concentration that is provided by a single dose of a second indicated drug. As a result, there appears to be virtually no benefit in combining the first drug and the second drug in a single dosage form because with a corresponding combination, the first drug would still provide the desired therapeutic effect when the other drug has ceased to be effective and would have to be administered again.

Accordingly, it would be desirable if patients suffering from symptoms for which a first drug is indicated would also obtain relief, over a similar time period, from one or more symptoms for which one or more second drugs are indicated, by administering a single dose of a dosage form such as, e.g., a tablet, liquid, syrup, suspension, gel, capsule, suppository and the like. In other words, it would be desirable to have available a dosage form which provides the therapeutic effects of drugs with significantly different pharmacokinetic properties over a similar period of time.

### SUMMARY OF THE INVENTION

The present invention provides a pharmaceutical dosage form which comprises a first drug and a second drug, both of which are selected from decongestants, antitussives, expectorants, analgesics and antihistamines and preferably belong to different classes of drugs. The dosage form provides a plasma concentration within the therapeutic range of the second drug over a period which is coextensive with at least about 70% of the period over which the dosage form provides a plasma concentration within the therapeutic range of the first drug.

In one aspect of the dosage form, the first drug may comprise a decongestant. By way of non-limiting example, the decongestant may comprise phenylephrine and/or pseudoephedrine and/or one or more pharmaceutically acceptable salts of one or both of two decongestants.

In another aspect of the dosage form, the first drug may comprise an antitussive. By way of non-limiting example, the antitussive may comprise one or more of a morphine derivative such as, e.g., codeine, dihydrocodeine, hydrocodone and hydromorphone, dextromethorphan, carbetapentane, chlophedianol, benzonatate, caramiphen, noscapine and/or one or more pharmaceutically acceptable salts of one or more of these antitussive drugs.

In another aspect of the dosage form, the first drug may comprise an antihistamine. Non-limiting examples of antihistamines for use in the present invention include acrivastine, astemizole, azatadine, azelastine, bromodiphenhydramine, brompheniramine, carbinoxamine, carebastine, cetirizine, chlophedianol, chlorcyclizine, clemastine, chlorothen, chlorpheniramine, cyclizine, cyproheptadine, descarboethoxyloratadine, desloratadine, dexbrompheniramine, dexchlorpheniramine, dimethindene, diphenhydramine, diphenylpyraline, doxylamine, ebastine, epanastine, efletirizine, fexofenadine, hydroxyzine, isothipendyl, ketotifen, loratadine, meclizine, methapyrilene, mizolastine, mequitazine, mianserin, montelukast, noberastine, norastemizole, picumast, phenindamine, pheniramine, phenyltoloxamine, promethazine, prophenpyridamine, pyrilamine, temelastine, terfenadine, thenyldiamine, thonzylamine, trimeprazine, tripelennamine, triprolidine, and pharmaceutically acceptable salts thereof. By way of non-limiting example, the antihistamine may comprise at least one of promethazine, diphenhydramine, chlorpheniramine, carbinoxamine and pharmaceutically acceptable salts thereof.

In yet another aspect of the dosage form, the first drug may comprise an analgesic. By way of non-limiting example, the analgesic may comprise one or more of aspirin, acetaminophen, ibuprofen, ketoprofen, naproxen, sodium naproxen, meloxicam, hydrocodone, oxycodone, morphine, meperidine and fentanyl.

In a still further aspect of the dosage form of the present invention, the first drug may comprise an expectorant such as, e.g., guaifenesin and/or a pharmaceutically acceptable salt thereof.

In another aspect of the dosage form of the present invention, the second drug may comprise a decongestant and/or an antitussive and/or an antihistamine and/or an analgesic and/or an expectorant. Non-limiting specific examples of these drugs include those which are given above as non-limiting examples of the first drug.

In another aspect of the dosage form, the plasma half-life of the second drug may differ from the plasma half-life of the first drug by at least about 2 hours, e.g., by at least about 3 hours, or by at least about 4 hours.

In yet another aspect of the dosage form of the present invention, the plasma concentration within the therapeutic range of the second drug may be coextensive with at least about 80%, e.g., at least about 90%, or at least about 95%, of the period of a plasma concentration within the therapeutic range of the first drug.

In a still further aspect of the dosage form, the dosage form may comprise a tablet such as, e.g., a bi-layered tablet. By way of non-limiting example, the tablet may comprise a matrix which comprises the first drug or the second drug and has dispersed therein particles which comprise the other one of the first drug and the second drug.

In another aspect of the dosage form, the dosage form may comprise a solution or a suspension.

The present invention also provides a bi-layered tablet which comprises a first layer and a second layer. The first layer comprises a first drug which is selected from decongestants, antitussives, expectorants, analgesics and antihistamines, and the second layer comprising a second drug which is also selected from decongestants, antitussives, expectorants, analgesics and antihistamines but is different from the first drug and preferably belongs to a different class than the first drug. The bi-layered tablet provides a plasma concentration within the therapeutic range of the second drug over a period which is coextensive with at least about 70% of the period over which the bi-layered tablet provides a plasma concentration within the therapeutic range of the first drug.

In one aspect of the bi-layered tablet of the present invention, the first layer and/or the second layer may comprise at least one drug that is selected from phenylephrine, pseudoephedrine, codeine, dihydrocodeine, hydrocodone, dextromethorphan, carbetapentane, chlophedianol, benzonatate, caramiphen, noscapine, guaifenesin, acrivastine, astemizole, azatadine, azelastine, bromodiphenhydramine, brompheniramine, carbinoxamine, carebastine, cetirizine, chlophedianol, chlorcyclizine, clemastine, chlorothen, chlorpheniramine, cyclizine, cyproheptadine, descarboethoxyloratadine, desloratadine, dexbrompheniramine, dexchlorpheniramine, dimethindene, diphenhydramine, diphenylpyraline, doxylamine, ebastine, epanastine, efletirizine, fexofenadine, hydroxyzine, isothipendyl, ketotifen, loratadine, meclizine, methapyrilene, mizolastine, mequitazine, mianserin, montelukast, noberastine, norastemizole, picumast, phenindamine, pheniramine, phenyltoloxamine, promethazine, prophenpyridamine, pyrilamine, temelastine, terfenadine, thenyldiamine, thonzylamine, trimeprazine, tripelennamine, triprolidine and pharmaceutically acceptable salts thereof, aspirin, acetaminophen, ibuprofen, ketoprofen, naproxen, sodium naproxen, meloxicam, hydrocodone, oxycodone, morphine, meperidine and fentanyl.

In another aspect of the bi-layered tablet, the period of a plasma concentration within the therapeutic range of the second drug may be coextensive with at least about 80%, e.g., at least about 90%, or at least about 95%, of the period of a plasma concentration within the therapeutic range of the first drug.

In yet another aspect of the bi-layered tablet, the plasma half-life of the second drug may differ from the plasma half-life of the first drug by at least about 2 hours, for example, by at least about 3 hours.

In a still further aspect of the bi-layered tablet, the first or the second layer may be an immediate release layer, or the first and the second layers may both be controlled release layers.

The present invention also provides a multi-layered tablet which comprises at least a first layer and a second layer. The first layer comprises a first drug which is selected from decongestants, antitussives, expectorants, analgesics and antihistamines and the second layer is a controlled release layer and comprises a second drug which is selected from decongestants, antitussives, expectorants, analgesics and antihistamines and is different from the first drug and preferably belongs to a different class than the first drug.

In one aspect of the multi-layered tablet, the first layer may be an immediate release layer and/or the second layer may be a controlled release layer.

In another aspect of the multi-layered tablet, the second drug may have a plasma half-life which differs from the plasma half-life of the first drug by at least about 2 hours, for example, by at least about 3 hours.

In yet another aspect of the multi-layered tablet, the tablet may provide a plasma concentration within the therapeutic range of the second drug over a period which is coextensive with at least about 90% of the period over which the tablet provides a plasma concentration within the therapeutic range of the first drug.

In a still further aspect of the multi-layered tablet, the layers thereof may be discrete zones which are arranged adjacent to each other, or one of the first and second layers may be partially or completely surrounded by the other one of the first and second layers.

The present invention also provides a pharmaceutical dosage form which comprises a first drug which is selected from antihistamines and has a first plasma half-life, and a second drug which is selected from decongestants, antitussives, analgesics and expectorants and has a second plasma half-life that differs from the first plasma half-life by at least about 3 hours. The dosage form provides a plasma concentration within the therapeutic range of the second drug over a period which is coextensive with at least about 80% of the period over which the dosage form provides a plasma concentration within the therapeutic range of the first drug.

In one aspect of the dosage form, the first plasma half-life may be longer by at least about 4 hours, e.g., longer by at least about 6 hours, than the second plasma half-life. In another aspect, the first plasma half-life may be at least about 8 hours.

In another aspect of the dosage form of the present invention, the period of a plasma concentration within the therapeutic range of the at least one second drug may be coextensive with at least about 90%, e.g., at least about 95%, or about 100% of the period of a plasma concentration within the therapeutic range of the first drug.

In yet another aspect, the dosage form may be associated with instructions to administer the dosage form three or fewer times per day, e.g., once or twice per day.

In a still further aspect, the dosage form may comprise a tablet.

In another aspect of the dosage form, the second drug may comprise a decongestant.

In another aspect, the antihistamine may be selected from acrivastine, astemizole, azatadine, azelastine, bromodiphenhydramine, brompheniramine, carbinoxamine, carebastine, cetirizine, chlorcyclizine, clemastine, chlorothen, chlorpheniramine, cyclizine, cyproheptadine, descarboethoxyloratadine, desloratadine, dexbrompheniramine, dexchlorpheniramine, dimethindene, diphenhydramine, diphenylpyraline, doxylamine, ebastine, epanastine, efletirizine, fexofenadine, hydroxyzine, isothipendyl, ketotifen, loratadine, meclizine, methapyrilene, mizolastine, mequitazine, mianserin, montelukast, noberastine, norastemizole, picumast, phenindamine, pheniramine, phenyltoloxamine, promethazine, prophenpyridamine, pyrilamine, temelastine, terfenadine, thenyldiamine, thonzylamine, trimeprazine, tripelennamine, triprolidine, and pharmaceutically acceptable salts thereof.

The present invention also provides dosage forms as set forth above, including the various aspects thereof, which dosage forms are substantially free of antihistamines (and/or contain less than therapeutically effective amounts thereof).

The present invention also provides a method of alleviating a condition which can be alleviated by administration of an antihistamine and at least one other condition which can be alleviated by administration of a drug which is at least one of a decongestant, an antitussive, an expectorant and an analgesic, wherein the method comprises administering a dosage form of the present invention, including the various aspects thereof, to a subject in need thereof.

The present invention also provides a process of making a pharmaceutical dosage form of the present invention, wherein the method comprises preparing a first composition which comprises the first drug and a second composition which comprises the second drug, and combining the first and the second compositions, for example, by using a tablet press.

The pharmaceutical dosage forms of the present invention comprise at least two drugs, i.e., a first drug and a second drug. It is noted that the terms “a first drug” and “a second drug” do not exclude, but rather include the presence of more than one first or second drug. For example, the dosage form may comprise one first drug and two or more (e.g., three, four, five, etc.) second drugs. In this case, the dosage form desirably provides plasma concentrations within the therapeutic ranges of all of the second drugs over periods of time which individually are coextensive with at least about 70% (e.g., at least about 80%, at least about 90%, at least about 95%, or at least about 100%) of the period of time over which the dosage form provides a plasma concentration within the therapeutic range of the first drug. Moreover, in cases where the dosage form comprises more than one first drug and more than one second drug, the first drug with the longest period of a plasma concentration with the therapeutic range is the standard, i.e., preferably all other drugs that are present in the dosage form show plasma concentrations within the therapeutic ranges thereof over periods of time which are coextensive with at least about 70% (e.g., at least about 80%, at least about 90%, at least about 95%, or at least about 100%) of the period for the first drug with the longest period of a plasma concentration with the therapeutic range.

The pharmaceutical dosage form which constitutes one aspect of the present invention comprises first and second drugs which may be present in the form of a pharmaceutically acceptable salt thereof. The term “pharmaceutically acceptable salt” as used herein and in the appended claims refers to those salts of a particular drug that are not substantially toxic at the dosage administered to achieve the desired effect and do not independently possess significant pharmacological activity. The salts included within the scope of this term are pharmaceutically acceptable acid addition salts of a suitable inorganic or organic acid. Non-limiting examples of suitable inorganic acids are, for example hydrochloric, hydrobromic, sulfuric and phosphoric acids. Non-limiting examples of suitable organic acids include carboxylic acids, such as acetic, propionic, tannic, glycolic, lactic, pyruvic, malonic, succinic, fumaric, malic, tartaric, citric, cyclamic, ascorbic, maleic, hydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acids, as well as sulfonic acids, such as methanesulfonic, ethanesulfonic, and β-hydroxyethanesulfonic acids.

Non-limiting examples of drugs for use in the present invention in the form of their pharmaceutically acceptable salts include codeine phosphate, codeine sulfate, hydrocodone bitartrate, dihydrocodeine bitartrate, carbetapentane citrate, pseudoephedrine hydrochloride, phenephrine hydrochloride, azatadine maleate, bromodiphenhydramine HCl, brompheniramine maleate, carbinoxamine maleate, cetirizine HCl, chlorcyclizine HCl, clemastine fumarate, chlorothen citrate, chlorpheniramine maleate, dimethindene maleate, diphenhydramine HCl, fexofenadine HCl, hydroxyine HCl, isothipendyl HCl (theruhistin), methapyrilene fumarate, methapyrilene HCl, montelukast sodium, phenindamine tartrate, pheniramine maleate, phenyltoloxamine citrate, promethazine hydrochloride, prophenpyridarnine maleate, pyrilamine maleate, thenyldiamine HCl, trimeprazine tartrate, tripelennamine HCl and triprolidine HCl.

The dosage form of the present invention will usually provide a plasma concentration within the therapeutic range of the second drug over a period which is coextensive with (overlaps) at least about 70%, more preferred at least about 80%, e.g., at least about 90%, at least about 95%, or about 100%, of the period over which the dosage form provides a plasma concentration within the therapeutic range of the first drug. The term “therapeutic range” as used herein and in the appended claims refers to the range of drug levels (including active metabolite levels) within which most patients will experience a significant therapeutic effect (including alleviation of symptoms) without an undesirable degree of adverse reactions. It is noted that the term “coextensive with” does not exclude, but rather includes, cases where a part of the period over which the plasma concentration of the second drug (and/or active metabolites thereof) is within the therapeutic range is outside the period over which the plasma concentration of the first drug is within the therapeutic range. In other words, even if the corresponding period for the second drug is to overlap, for example, 70% of the corresponding period of the first drug, a certain percentage (preferably not more than about 30%, e.g., not more than about 20%, not more than about 10% or even not more than about 5%) of the total period over which the plasma concentration of the second drug is within the therapeutic range may be outside the period over which the plasma concentration of the first drug is within the therapeutic range.

The period over which the therapeutic range of a particular drug may be provided in a given case depends, at least in part, on the plasma half-life of the drug and/or active metabolites thereof. The term “plasma half-life” as used herein and in the appended claims refers to the time required for the plasma drug concentration to decline by 50%. The shorter the plasma half-life of a particular drug, the shorter will be the period within the therapeutic range of the drug which is provided by a single administered dose of the drug. In one preferred aspect of the dosage form of the present invention, the plasma half-life of the second drug will be shorter than the plasma half-life of the first drug by at least about 2 hours, e.g., by at least about 3 hours, by at least about 4 hours, by at least about 5 hours, by at least about 6 hours, by at least about 8 hours, or even by at least about 10 hours.

A preferred, although non-limiting, embodiment of the dosage form of the present invention is a tablet, in particular, a bi-layered tablet. Non-limiting examples of other embodiments of the dosage form of the invention are capsules, pills, chewable tablets, suspensions, solutions, syrups, and suppositories.

The bi-layered tablet which forms one of the aspects of the present invention comprises two layers. The first layer comprises a first drug which is selected from decongestants, antitussives, expectorants, analgesics and antihistamines. The second layer comprises a second drug which is different from the first drug and is selected from decongestants, antitussives, expectorants, analgesics and antihistamines. Specific and non-limiting examples of such drugs are given above. Particularly for the treatment of conditions which are associated with thick tenacious mucus exudates it is preferred for the bi-layered tablet or any other dosage form of the present invention to be substantially antihistamine-free, i.e., neither the first nor the second drug comprise more than insignificant amounts of antihistamines or any other drugs which have a substantial drying action.

The layers of the bi-layered tablet of the present invention may individually be immediate release layers or controlled release layers. The term “controlled release layer” as used herein and in the appended claims refers to any layer that is not an immediate release layer, i.e., does not release all of the active ingredients contained therein within a relatively short time (for example, within less than 1 hour, e.g., less than 0.5 hours, following ingestion of the dosage form). Accordingly, this term is a generic term which encompasses, e.g., sustained (extended) release layers, pulsed release layers, delayed release layers, and the like. Preferably, the controlled release layer releases the one or more drugs contained therein continuously or intermittently and, preferably, in approximately equal amounts per time unit (e.g., zero order release rate), over an extended period of time such as, e.g., at least about 2 hours, at least about 3 hours, at least about 4 hours, or at least about 6 hours. The desirable length of the time period of continuous or intermittent (e.g., pulsed) release depends, inter alia, on the plasma half-life of the drug and/or an active metabolite thereof.

The first and second layers of the bi-layered tablet of the present invention will usually contain the first and second drugs in amounts which are commensurate with the intended duration of action but will not give rise to overdosing when present in the intended form (e.g., a particular pharmaceutically acceptable salt) and the intended release matrix (e.g., immediate release, controlled release, etc.). In this regard, it is noted that a drug may be present in both the first layer and the second layer (and any additional layer, if present), for example, in order to obtain an as fast as possible release and, thus action of the drug (e.g., by using an immediate release matrix) and to at the same time extend the duration of the action of the drug (e.g., by using a controlled release matrix that releases the drug at a lower rate and/or at a later time than the immediate release layer). In the following, preferred ranges of amounts of selected drugs for use in the bi-layered tablet and other dosage forms of the present invention are given for illustrative purposes only.

Promethazine: at least about 0.1 mg, e.g., at least about 5 mg, at least about 6 mg, at least about 8 mg, at least about 12 mg, or at least about 25 mg, but not more than about 90 mg, e.g., not more than about 75 mg, not more than about 70 mg, not more than about 60 mg, or not more than about 50 mg of promethazine hydrochloride, or an equivalent amount (on a molar basis) of promethazine and/or any other pharmaceutically acceptable salt thereof.

Chlorpheniramine: at least about 0.1 mg, e.g., at least about 2 mg, or at least about 4 mg, but not more than about 16 mg, e.g., not more than about 12 mg of chlorpheniramine maleate, or an equivalent amount of chlorpheniramine and/or any other pharmaceutically acceptable salt thereof.

Carbinoxamine: at least about 0.1 mg, e.g., at least about 6 mg, but not more than about 32 mg, e.g., not more than about 24 mg of carbinoxamine maleate, or an equivalent amount of carbinoxamine and/or any other pharmaceutically acceptable salt thereof.

Diphenhydramine: at least about 10 mg, e.g., at least about 15 mg, at least about 20 mg, at least about 40 mg, at least about 70 mg, or at least about 90 mg, but not more than about 200 mg, e.g., not more than about 150 mg, not more than about 120 mg, or not more than about 100 mg of diphenhydramine hydrochloride, or an equivalent amount of diphenhydramine and/or any other pharmaceutically acceptable salt thereof.

Carbetapentane: at least about 1 mg, e.g., at least about 5 mg, at least about 10 mg, at least about 25 mg, or at least about 50 mg, but not more than about 120 mg, e.g., not more than about 100 mg, not more than about 70 mg, or not more than about 60 mg, of carbetapentane citrate, or an equivalent amount of carbetapentane and/or any other pharmaceutically acceptable salt thereof.

Codeine: at least about 1 mg, e.g., at least about 10 mg, at least about 25 mg, or at least about 30 mg, but not more than about 120 mg, e.g., not more than about 80 mg, not more than about 60 mg, or not more than about 45 mg, of codeine phosphate, or an equivalent amount of codeine and/or any other pharmaceutically acceptable salt thereof.

Dihydrocodeine: at least about 1 mg, e.g., at least about 5 mg, but not more than about 30 mg, e.g., not more than about 20 mg, of dihydrocodeine bitartrate, or an equivalent amount of dihydrocodeine and/or any other pharmaceutically acceptable salt thereof.

Hydrocodone: at least about 1 mg, e.g., at least about 5 mg, but not more than about 20 mg, e.g., not more than about 15 mg, of hydrocodone bitartrate, or an equivalent amount of hydrocodone and/or any other pharmaceutically acceptable salt thereof.

Phenylepherine: at least about 1 mg, e.g., at least about 10 mg, or at least about 15 mg, but not more than about 125 mg, e.g., not more than about 100 mg, not more than about 90 mg, not more than about 75 mg, not more than about 50 mg, or not more than about 25 mg of phenylephrine hydrochloride, or an equivalent amount of phenylephrine and/or any other pharmaceutically acceptable salt thereof.

Pseudoephedrine: at least about 1 mg, e.g., at least about 10 mg, at least about 25 mg, or at least about 50 mg, but not more than about 240 mg, e.g., not more than about 150 mg, not more than about 100 mg, or not more than about 70 mg of pseudoephedrine hydrochloride, or an equivalent amount of pseudoephedrine and/or any other pharmaceutically acceptable salt thereof.

Guaifenesin: at least about 1 mg, e.g., at least about 10 mg, at least about 25 mg, at least about 50 mg, or at least about 100 mg, but not more than about 2400 mg, e.g., not more than about 1600 mg, not more than about 1500 mg, not more than about 1200 mg, not more than about 1000 mg, not more than about 600 mg, or not more than about 500 mg of guaifenesin, or an equivalent amount of a pharmaceutically acceptable salt thereof.

Acetaminophen: at least about 10 mg, e.g., at least about 50 mg, or at least about 100 mg, but not more than about 1000 mg, e.g., not more than about 500 mg, or not more than about 250 mg of acetaminophen.

Another aspect of the present invention is a multi-layered tablet which comprises at least a first layer and a second layer, but may optionally comprise a third, fourth, fifth, etc. layer. The first layer may be an immediate release layer or a controlled release layer, depending on the drug(s) contained therein and the desired release characteristics thereof, and comprises at least one first drug which is selected from decongestants, antitussives, expectorants, analgesics and antihistamines. The mandatory second layer may also be an immediate release layer or a controlled release layer, depending on the drug(s) contained therein and the desired release characteristics thereof, and comprises at least one second drug which is different from the first drug and is selected from decongestants, antitussives, expectorants, analgesics and antihistamines. If more than two drugs are to be incorporated in the tablet, the first and/or the second layer may contain all of the additional drugs. Alternatively, separate additional layers may be provided for the additional drugs, for example, in cases where it would be difficult to design a controlled release layer which provides a desired release rate for both the first or second drug and/or the additional drug(s). Of course, a fourth, fifth, etc. layer may be provided for, e.g., a third or fourth additional drug, and so on. Alternatively and by way of non-limiting example, the second and a third layer may contain the same drug or drugs, but in different (relative) concentrations and/or incorporated in a different controlled release formulation.

The multi-layered tablet of the present invention will usually be made up of two or more distinct layers or discrete zones of granulation compressed together with the individual layers lying on top of one another. Layered tablets have the appearance of a sandwich because the edges of each layer or zone are exposed. Such conventional layered tablets are generally prepared by compressing a granulation onto a previously compressed granulation. The operation may be repeated to produce multi-layered tablets of more than two layers. In a preferred embodiment of the multi-layered tablet of the present invention, the tablet consists of two layers.

It is to be noted that it is not necessary for the two or more individual layers of the multi-layered tablet of the present invention to lie on top of one another. By way of non-limiting example, a second layer (e.g., sustained release layer) may be partially or completely surrounded by a first layer (e.g., an immediate release layer). For example, the second layer may be coated with the first layer. In the case of three layers, for example, the third layer may be partially or completely coated with the second layer, which in turn may be partially or completely coated with the first layer. Of course, these are but a few examples of the many different ways in which the various layers of the multi-layered tablet of the present invention can be arranged relative to each other. Moreover, it is to be understood that the tablets of the present invention are not limited to such multi-layered tablets. By way of non-limiting example, the tablet may comprise an immediate release matrix which comprises one or more first drugs, which matrix has dispersed therein particles of one or more sustained release formulations which have the one or more second drugs and any of the additional desired drug(s) incorporated therein.

In another aspect of the multi-layered tablet, the at least one drug in the second layer (and/or in the additional layers) may have a plasma half-life which is shorter by at least about 3 hours, e.g., shorter by at least about 4 hours, or shorter by at least about 6 hours, than the plasma half-life of the first drug or the first drug with the longest plasma half-life, respectively.

In another aspect of the multi-layered tablet, the tablet may provide a plasma concentration within a therapeutic range of the drug(s) in the second layer over a period which is coextensive with at least about 80%, e.g., at least about 90%, of the period over which the multi-layered tablet provides a plasma concentration within the therapeutic range of the drug(s) in the first layer.

Another aspect of the present invention is formed by a liquid dosage form, preferably a suspension, which comprises (a) a first drug which is selected from decongestants, expectorants, antitussives, analgesics and antihistamines and (b) a second drug which is selected from decongestants, expectorants, antitussives, analgesics and antihistamines and is different from the first drug. This liquid dosage form provides a plasma concentration within the therapeutic range of component (b) over a period which is coextensive with at least about 70%, preferably at least 80%, e.g., at least 90%, of the period over which the liquid dosage form provides a plasma concentration within the therapeutic range of component (a). This may be accomplished in various ways. By way of non-limiting example, component (b) may be incorporated into a solid controlled release formulation. For example, particles of component (b) may be provided with a controlled release coating (e.g. a controlled release coating comprising an organic polymer such as, e.g., a polyacrylate). This formulation may then be comminuted, if necessary, in an appropriate manner (e.g., by milling) to form particles of a size which is small enough to be suitable for being suspended in a pharmaceutically acceptable liquid carrier. Component (a), on the other hand, may be used as such or incorporated in a solid immediate release formulation, comminuted and incorporated into the liquid carrier as well. A non-limiting example of a corresponding procedure is described in the Examples below.

Prior to incorporating components (a) and (b) into a pharmaceutically acceptable liquid carrier to form a liquid dosage form according to the present invention, at least a part of component (a) and/or at least a part of component (b) may be converted into a complex with a complexing agent. Non-limiting examples of suitable complexing agents comprise ion-exchange resins such as, e.g., (sodium) polystyrene sulfonate.

The dosage forms of the present invention can be manufactured by processes which are well known to those of skill in the art. For example, for the manufacture of bi-layered tablets, the active ingredients may be dispersed uniformly into a mixture of excipients, for example, by high shear granulation, low shear granulation, fluid bed granulation, or by blending for direct compression. Excipients may include diluents, binders, disintegrants, dispersants, lubricants, glidants, stabilizers, surfactants and colorants. Diluents, also termed “fillers”, are typically used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders impart cohesive qualities to a tablet formulation and are used to ensure that a tablet remains intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants facilitate tablet manufacture; non-limiting examples thereof include magnesium stearate, calcium stearate, stearic acid, glyceryl behenate, and polyethylene glycol. Disintegrants facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc and the like. Stabilizers inhibit or retard drug decomposition reactions, including oxidative reactions. Surfactants may be anionic, cationic, amphoteric or nonionic. If desired, the tablets may also contain minor amounts of nontoxic auxiliary substances such as pH buffering agents, preservatives, e.g., antioxidants, wetting or emulsifying agents, solubilizing agents, coating agents, flavoring agents, and the like.

Extended/sustained release formulations may be made by choosing the right combination of excipients that slow the release of the active ingredients by coating or temporarily bonding or decreasing the solubility of the active ingredients. Examples of these excipients include cellulose ethers such as hydroxypropylmethylcellulose (e.g., Methocel K4M), polyvinylacetate-based excipients such as, e.g., Kollidon SR, and polymers and copolymers based on methacrylates and methacrylic acid such as, e.g., Eudragit NE 30D.

There are several commercially available tablet presses capable of making bi-layered tablets. For example, Manesty RotaPress Diamond, a 45 station D tooling press, is capable of making bi-layered tablets described in this application. Non-limiting examples of presses for the manufacture of bi-layered tablets include Fette America Model No. PT 3090; Maneklal Global Exports (Mumbai, India) Models JD and DH series; Niro Pharma Systems, Model R292F; and Korsch AG Models XL 800 and XL 400.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fumdamental understanding of the present invention, the description making apparent to those skilled in the art how the several forms of the present invention may be embodied in practice.

### EXAMPLE 1

### Liquid Formula

A liquid dosage form in accordance with the present invention which comprises promethazine hydrochloride, dihydrocodeine bitartrate and phenylephrine hydrochloride is illustrated as follows:

| Ingredients | Per 5 mL | | Per 425 L | |
|---|---|---|---|---|
| Promethazine Hydrochloride USP | 12.5 | mg | 1.063 | kg |
| Dihydrocodeine Bitartrate USP | 10.0 | mg | 0.850 | kg |
| Phenylepherine Hydrochloride USP | 10.0 | mg | 0.850 | kg |
| Methyl Paraben USP | 9.0 | mg | 0.765 | kg |
| Propyl Paraben USP | 1.0 | mg | 0.085 | kg |
| Propylene Glycol USP | 259 | mg | 22.016 | kg |
| Saccharin Sodium USP | 3.18 | mg | 0.270 | kg |
| Citric Acid USP | 5.0 | mg | 0.425 | kg |
| Strawberry Flavor | 10 | mg | 0.850 | kg |
| Banana Flavor | 10 | mg | 0.850 | kg |
| Sorbitol Solution 70% USP | 3212.5 | mg | 273.1 | kg |
| Purified Water, as required to q.s. to | 5.0 | mL | 425 | L |

Manufacturing process for 425 L batch size: In a suitably sized stainless steel vessel, dissolve methyl paraben and propyl paraben in approximately 50 L of warm (about 45° C.), purified water. Add about half of the propylene glycol and mix for about 1 hr. In a separate 1000 L stainless steel tank equipped with a suitably sized agitator, add about 50 L of purified water. With the agitator on, add phenylephrine hydrochloride, promethazine hydrochloride, saccharin sodium and citric acid and dissolve. Add the previously prepared paraben/propylene glycol solution to the 1000 L tank. Rinse the first vessel with about 2 L of water and transfer the rinsate to the 1000 L tank. Add the remaining propylene glycol to a suitably sized stainless steel vessel and dissolve the strawberry and banana flavors. Transfer this to the 1000 L tank. Rinse the container with 2 L of purified water and transfer to the 1000 L tank. With the agitator on, add the sorbitol solution 70% to the 1000 L tank. In a suitably sized stainless steel vessel, dissolve the dihydrocodeine bitartrate in about 5 L of purified water and transfer to the 1000 L tank. Rinse the container with about 2 L of purified water and transfer to the 1000 L tank. Stop the agitator and let the solution stand for 15 minutes. QS to 425 L with purified water. Filter product through a 1 micron filter and fill in appropriately sized containers.

To make products with other antihistamines, decongestants, antitussives, or expectorants, one or more combinations of each of the ingredients in a range as described in Table 1 below can be made depending on the specific therapeutic effect desired.

### EXAMPLE 2

### Suspension Formula

A suspension formula in accordance with the present invention which comprises promethazine hydrochloride and phenylephrine tannate is illustrated as follows:

| | g/100 mL = | | kg/batch = | |
|---|---|---|---|---|
| Ingredients | 120 g | | 1000 kg | |
| Promethazine Hydrochloride | 0.500 | | 4.167 | |
| Phenylepherine Tannate | 0.800 | | 6.667 | |
| Silica, colloidal anhydrous, NF | 1.73 | | 14.417 | |
| Hydroxyethylcellulose, NF | 0.05 | | 0.417 | |
| Sorbitol Solution 70% (non-crystallizing), | 34.00 | | 283.333 | |
| NF | | | | |
| Glycerol | 14.75 | | 122.917 | |
| Xylitol, NF | 16.00 | | 133.333 | |
| Sodium Citrate, USP | 2.00 | | 16.667 | |
| Saccharin Sodium cryst., USP, | 0.01 | | 0.083 | |
| Sodium Benzoate, NF | 0.15 | | 1.250 | |
| Citric Acid Monohydrate, USP | 0.16 | | 1.333 | |
| Strawberry Flavor | 0.15 | | 1.250 | |
| Banana Flavor | 0.15 | | 1.250 | |
| Purified Water | 49.55 | | 412.917 | |
| Total Amount | 120.000 | g | 1000.000 | kg |

Manufacturing process for 1000 kg batch: In a suitably sized stainless steel vessel, dissolve saccharin sodium, sodium benzoate, citric acid, and sodium citrate in approximately 50 L of warm (about 45 deg C.), purified water. In another large stainless steel drum mix the silica, promethazine hydrochloride and micronized phenylephrine tannate until a uniform and consistent mixture is obtained. In a separate 1000 L stainless steel tank equipped with a suitably sized homogenizer/disperser add about 100 L of purified water. With the homogenizer on, add the silica mixture containing phenylephrine tannate and promethazine hydrochloride. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes. Filter product through a 10 micron filter and fill in appropriately sized containers.

To make products with other antihistamines, decongestants, antitussives, or expectorants, one or more combinations of each of the ingredients in a range as described in Table 1 below can be made depending on the specific therapeutic effect desired.

### EXAMPLE 3

### Bi-Layered Tablet (Direct Compression)

A bi-layered tablet in accordance with the present invention which comprises promethazine hydrochloride in one layer and phenylephrine hydrochloride and chlorpheniramine maleate in the other layer is illustrated as follows:

| | | |
|---|---|---|
| | Weight/tablet | Weight/1 kg batch |
| Ingredients | (mg) | (in grams) |

Layer 1 (Immediate release)

| | | |
|---|---|---|
| Promethazine Hydrochloride | 25.0 | 45.5 |
| Silicified Microcrystalline | 114.0 | 207.3 |
| Cellulose | | |
| Sodium Starch Glycolate | 10.0 | 18.2 |
| Magnesium Stearate | 1.0 | 1.8 |

Layer 2 (Sustained release)

| | | |
|---|---|---|
| Phenylepherine HCl | 20.0 | 36.4 |
| Chlorpheniramine Maleate | 8.0 | 14.5 |
| Lactose Monohydrate | 50.0 | 90.9 |
| Dicalcium Phosphate | 50.0 | 90.9 |
| Kollidon SR | 252.0 | 458.2 |
| Stearic acid | 15.0 | 27.3 |
| Magnesium Stearate | 5.0 | 9.1 |
| Total | 550.0 | 1000.0 |

Manufacturing Process

(a) Immediate release layer: Screen all ingredients through a USP sieve size # 30. Blend promethazine hydrochloride (45.5 gms), silicified microcrystalline cellulose (207.3 gms) and sodium starch glycolate (18.2 gms) in a twin shell blender for 20 minutes. Add magnesium stearate (1.8 gms), which acts as a lubricant, to the above blend and mix for 3 minutes.

(b) Sustained release layer: Screen all ingredients through a USP sieve size # 30. Preblend a portion of the Kollidon SR (145 gms) and all the chlorpheniramine maleate (14.5 gms) for 15 minutes. Add the remaining Kollidon SR (313.2 gms), phenylephrine hydrochloride (36.4 gms), lactose monohydrate (90.9 gms) and dicalcium phosphate (90.9 gms) to the above preblend and mix for an additional 20 minutes. Add stearic acid (27.3 gms) and magnesium stearate (9.1 gms) to the above blend and mix for three minutes.

Manufacture bi-layered tablets using a rotary bi-layer tablet press where in each tablet the immediate release layer is 150 mgs and the sustained release layer is 400 mgs.

By using the process described above, a bi-layered tablet of the following composition may be manufactured by using direct compression:

| | |
|---|---|
| | Weight/tablet |
| Ingredients | (mgs) |

Layer 1 (Immediate Release)

| | |
|---|---|
| Promethazine Hydrochloride | 50 |
| Silicified Microcrystalline Cellulose | 133.5 |
| Sodium Starch Glycholate | 15 |
| Magnesium Stearate | 1.5 |

Layer 2 (Sustained Release)

| | |
|---|---|
| Phenylephrine HCl | 20 |
| Chlorpheniramine Maleate | 8 |
| Lactose Monohydrate | 50 |
| Dicalcium Phosphate | 50 |
| Kollidon SR | 252 |
| Stearic Acid | 15 |
| Magnesium Stearate | 5 |
| Total | 600 |

### EXAMPLE 4

### Bi-Layered Tablet (Wet Granulation)

A bi-layered tablet in accordance with the present invention which comprises promethazine hydrochloride in one layer and pseudoephedrine hydrochloride and chlorpheniramine maleate in the other layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Promethazine Hydrochloride | 25.0 | 35.7 |
| Silicified Microcrystalline Cellulose | 111.0 | 158.6 |
| Povidone | 3.0 | 4.3 |
| Croscarmellose Sodium | 10.0 | 14.3 |
| Magnesium Stearate | 1.0 | 1.4 |

Layer 2 (Sustained release)

| | | |
|---|---|---|
| Pseudoephedrine HCl | 60.0 | 85.7 |
| Chlorpheniramine Maleate | 8.0 | 11.4 |
| Microcrystalline Cellulose (PH 102) | 30.0 | 42.9 |
| Lactose Monohydrate | 100.0 | 142.9 |
| Dicalcium Phosphate | 100.0 | 142.9 |
| Povidone | 15.0 | 21.4 |
| Methocel K4M Premium | 212.0 | 302.9 |
| Stearic Acid | 20.0 | 28.6 |
| Magnesium Stearate | 5.0 | 7.1 |
| Total | 700.0 | 1000.0 |

Manufacturing Process

(a) Immediate release layer: Screen all ingredients through a USP sieve size # 30. Blend promethazine hydrochloride (35.7 gms), silicified microcrystalline cellulose (158.6 gms) and croscarmellose sodium (14.3 gms) in a high shear mixer/granulator for 10 minutes. Granulate the above blend using a 30% povidone solution (4.3 gms povidone in 14.3 gms purified water). Dry the granulation until the loss on drying (LOD) is less than 2.0%. Screen the dried granulation through a USP sieve size # 14. Add the screened granulation and the prescreened magnesium stearate (1.4 gms) to the above blend and mix for 3 minutes.

(b) Sustained release layer: Screen all ingredients through a USP sieve size # 30. Blend the pseudoephedrine hydrochloride (87.5 gms), chlorpheniramine maleate (11.4 gms), microcrystalline cellulose PH 102 (42.9 gms), lactose monohydrate (142.9 gms), dicalcium phosphate (142.9gms), Methocel K4M Premium (302.9 gms) and stearic acid (28.6 gms) in a high shear mixer/granulator for 10 minutes. Granulate the above blend using a 30% povidone solution (21.4 gms povidone in 71.3 gms purified water). Dry the granulation till the LOD is less than 2.0%. Screen granules through a USP sieve size # 14. Add granules and the prescreened magnesium stearate (7.1 gms) to the above blend and mix for 3 minutes.

Manufacture bi-layered tablets using a rotary bi-layer tablet press where in each tablet the immediate release layer is 150 mgs and the sustained release layer is 550 mgs.

By using the process described above, a bi-layered tablet of the following composition may be manufactured by using wet granulation:

| | |
|---|---|
| | Weight/tablet |
| Ingredients | (mgs) |

Layer 1 (Immediate Release)

| | |
|---|---|
| Promethazine Hydrochloride | 50 |
| Silicified Microcrystalline cellulose | 129.5 |
| Povidone | 4 |
| Croscarmellose sodium | 15 |
| Magnesium Stearate | 1.5 |

Layer 2 (Sustained Release)

| | |
|---|---|
| Pseudoephedrine HCl | 60 |
| Chlorpheniramine Maleate | 8 |
| Microcrystalline Cellulose 102 | 30 |
| Lactose Monohydrate | 100 |
| Dicalcium Phosphate | 100 |
| Povidone | 15 |
| Hydroxypropylmethylcellulose | 212 |
| Stearic Acid | 20 |
| Magnesium Stearate | 5 |
| Total | 750 |

The above examples illustrate how to manufacture a bi-layered tablet containing an antihistamine, i.e., promethazine hydrochloride, in one layer and an antihistamine and/or a decongestant and/or an antitussive and/or an expectorant in the other layer. For the layer that does not contain promethazine hydrochloride, combinations of one or more of each of the non-limiting examples of possible ingredients in an exemplary range as described in the following Table 1 can be made depending on the specific therapeutic effect desired.

TABLE 1

| | | | Preferred | | OTC | |
|---|---|---|---|---|---|---|
| | Amount per | | Amount per | | Daily | |
| Active ingredient | Tablet | | Tablet | | Dosage | |
| ANTIHISTAMINES | | | | | | |
| Azelastine hydrochloride | 0.1-2.0 | mg | 0.125 | mg | | |
| Azatadine hydrochloride | 0.1-4.0 | mg | 1 | mg | | |
| Brompheniramine maleate | 0.1-64 | mg | 2-16 | mg | 24 | mg |
| Dexbrompheniramine maleate | 0.1-24 | mg | 3-6 | mg | 12 | mg |
| Carbinoxamine maleate | 0.1-16 | mg | 4 | mg | | |
| Cetirizine hydrochloride | 0.1-40 | mg | 5-10 | mg | | |
| Chlorcyclizine | 0.1-300 | mg | | | 75 | mg |
| Chlorpheniramine maleate | 0.1-64 | mg | 2-16 | mg | 24 | mg |
| Chlorpheniramine polistirex | 0.1-32 | mg | 4-8 | mg | | |
| Clemastine | 0.1-12 | mg | 0.5-2.68 | mg | | |
| Cyproheptadine | 0.1-16 | mg | 2-4 | mg | | |
| Dexchlorpheniramine maleate | 0.1-24 | mg | 2 | mg | 12 | mg |
| Cyproheptadine hydrochloride | 0.1-32 | mg | 2-4 | mg | | |
| Diphenhydramine hydrochloride | 0.1-300 | mg | 10-50 | mg | 300 | mg |
| Diphenhydramine citrate | 0.1-2000 | mg | | | 456 | mg |
| Bromodiphenhydramine | 0.1-200 | mg | 12.5-25 | mg | | |
| hydrochloride | | | | | | |
| Doxylamine succinate | 0.1-200 | mg | 12.5-25 | mg | 75 | mg |
| Fexofenadine hydrochloride | 0.1-720 | mg | 30-180 | mg | | |
| Hydroxyzine hydrochloride | 0.1-400 | mg | 10-100 | mg | | |
| Hydroxyzine pamoate | 0.1-400 | mg | 25-100 | mg | | |
| Loratadine | 0.1-80 | mg | 1-10 | mg | | |
| Desloratadine | 0.1-40 | mg | 5 | mg | | |
| Phenindamine tartrate | 0.1-750 | mg | | | 150 | mg |
| Pheniramine maleate | 0.1-750 | mg | | | 150 | mg |
| Pyrilamine maleate | 0.1-200 | mg | 25 | mg | 200 | mg |
| Terfenadine | | | | | | |
| Thenyldiamine | | | | | | |
| Thonzylamine | 0.1-3000 | mg | | | 600 | mg |
| Thymol | | | | | | |
| Tripelennamine hydrochloride | 0.1-400 | mg | 25-50 | mg | | |
| Triprolidine hydrochloride | 0.1-40 | mg | 1.25-5 | mg | 10 | mg |
| ANTITUSSIVES | | | | | | |
| Chlophedianol hydrochloride | 0.1-800 | mg | | | 100 | mg |
| Codeine | 0.1-240 | mg | 8.4-60 | mg | 120 | mg |
| Codeine phosphate | 0.1-240 | mg | 2.5-60 | mg | 120 | mg |
| Codeine sulfate | 0.1-480 | mg | | | 120 | mg |
| Dextromethorphan | 0.1-480 | mg | | | 120 | mg |
| Dextromethorphan hydrobromide | 0.1-240 | mg | 3.3-30 | mg | 120 | mg |
| Dextromethorphan polistirex | 0.1-240 | mg | 30 | mg | | |
| Diphenhydramine citrate | 0.1-1000 | mg | | | 228 | mg |
| Diphenhydramine hydrochloride | 0.1-400 | mg | 10-50 | mg | 150 | mg |
| Benzonatate | 0.1-800 | mg | 100-200 | mg | | |
| Hydrocodone bitartrate | 0.1-40 | mg | 1.66-10 | mg | | |
| Dihydrocodeine | 0.1-128 | mg | 16-32 | mg | | |
| Caramiphen edisylate | 0.1-160 | mg | 6.7-40 | mg | | |
| Carbetapentane tannate | 0.1-480 | mg | 30-60 | mg | | |
| Carbetapentane citrate | 0.1-160 | mg | 20 | mg | | |
| Hydromorphone | 0.1-8 | mg | 1 | mg | | |
| Noscapine | 0.1-200 | mg | | | | |
| EXPECTORANT | | | | | | |
| Guaifenesin | 0.1-2000 | mg | 50-1200 | | 2400 | mg |

### EXAMPLE 5

### Extended Release Suspension

| Ingredients | Amount/5 ml |
|---|---|
| Hydrocodone ion-exchange | Equivalent to 8 mgs Hydrocodone |
| complex | bitartarate |
| Dexchlorpheniramine ion-exchange | Equivalent to 4 mgs |
| complex | Dexchlorpheniramine maleate |
| Phenylephrine ion-exchange complex | Equivalent to 10 mgs |
| | Phenylephrine HCl |
| Eudragit ® L 100 | 0.2 to 2.8 grams |
| Glycerin | 315 mgs |
| Polysorbate 80 | 1.5 mgs |
| Carbomer (e.g., Carbopol ® 974) | 15 mgs |
| Methyl Paraben | 9 mgs |
| Propyl Paraben | 1 mgs |
| Artificial grape flavor | 5 mgs |
| FD&C red # 40 dye | 0.5 mgs |
| Water | q.s |

The formula described above serves as a non-limiting example. Any active drug which is in the form of a salt, such as promethazine hydrochloride, codeine phosphate, pseudoephedrine hydrochloride, morphine sulfate, or meperidine hydrochloride can be incorporated as an ion-exchange resin complex.

Procedure:

(1) Add the appropriate amount of sodium polystyrene sulphonate USP (e.g. Amberlite® IRP 69) to a hydrocodone bitartarate, dexchlorpheniramine maleate and phenylephrine HCl solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eugragit® L 100, Kollidon® MAE, Aquacoa®t cPD) and dry the granules.

(5) Mill the granules, if needed.

(6) To an appropriate amount of water add the following ingredients and dissolve: Carbomer (e.g., Carbopol® 974), glycerin, polysorbate 80, methyl paraben, propyl paraben, artificial grape flavor, FD&C red # 40 dye.

(7) Add milled granules.

(8) Add water to make up to a final volume.

(9) Agitate at suitable rate to avoid settling of the suspension and maintain a homogeneous product mixture.

(10) Fill in suitable containers ensuring that the product is homogeneous throughout the filling operation.

### EXAMPLE 6

### Bi-Layered Tablet (Wet Granulation)

A bi-layered tablet in accordance with the present invention which comprises guaifenesin in a first sustained release layer and codeine phosphate and pseudoephedrine hydrochloride in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Guaifenesin | 600.0 | 510.6 |
| Methocel K15M | 100.0 | 85.1 |
| Silicified Microcrystalline Cellulose | 50 | 42.6 |
| Eudragit NE | 42 | 35.7 |
| Magnesium Stearate | 8.0 | 6.8 |

Layer 2 (Sustained release)

| | | |
|---|---|---|
| Codeine Phosphate | 30.0 | 25.5 |
| Pseudoephedrine HCl | 120.0 | 102.1 |
| Microcrystalline Cellulose (PH 102) | 45.0 | 38.3 |
| Eudragit NE | 15.0 | 12.8 |
| Methocel K4M Premium | 140.0 | 119.1 |
| Stearic Acid | 20.0 | 17.0 |
| Magnesium Stearate | 5.0 | 4.3 |
| Total | 1175.0 | 1000.0 |

Procedure:

(a) Sustained release layer #1: Mix the guaifenesin, Methocel®K15M and silicified microcrystalline cellulose in a high shear mixer/granulator for 10 minutes. Granulate the above blend using a Eudragit®NE (30%). Dry the granulation until the LOD (weight loss on drying) is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, pseudoephedrine HCl, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 800 mgs and layer #2 is 375 mgs. Capsules may be manufactured by filling the same proportions into capsules.

### EXAMPLE 7

### Bi-Layered Tablet (Wet Granulation)

A bi-layered tablet in accordance with the present invention which comprises promethazine hydrochloride in an immediate release layer and codeine phosphate and pseudoephedrine hydrochloride in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Promethazine HCl | 25.0 | 37.0 |
| Silicified Microcrystalline Cellulose | 111.0 | 164.3 |
| Povidone | 3.0 | 4.4 |
| Croscarmellose Sodium | 10.0 | 14.8 |
| Magnesium Stearate | 1.0 | 1.5 |

Layer 2 (Sustained release)

| | | |
|---|---|---|
| Codeine Phosphate | 30.0 | 44.4 |
| Pseudoephedrine HCl | 120.0 | 177.6 |
| Microcrystalline Cellulose (PH 102) | 30.0 | 44.4 |
| Dicalcium Phosphate | 100.0 | 148.0 |
| Povidone | 15.0 | 22.2 |
| Methocel K4M Premium | 205.0 | 303.4 |
| Stearic Acid | 20.0 | 29.6 |
| Magnesium Stearate | 5.0 | 7.4 |
| Total | 675.0 | 1000.0 |

Procedure:

(a) Immediate release layer #1: Mix the promethazine HCl, silicified microcrystalline cellulose and croscarmellose sodium, in a high shear mixer/granulator for 10 minutes. Granulate the above blend using a 30% povidone solution (3.0 gms povidone in 10.0 gms purified water). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Mix the codeine phosphate, pseudoephedrine HCl, microcrystalline cellulose PH 102, dicalcium phosphate, Methocel K4M Premium and stearic acid in a high shear mixer/granulator for 10 minutes. Granulate the above blend using a 30% povidone solution (15.0 gms povidone in 50.0 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 150 mgs and layer #2 is 525 mgs. Capsules may be manufactured by filling the same proportions into capsules.

### EXAMPLE 8

### Bi-Layered Tablet (Wet Granulation)

A bi-layered tablet in accordance with the present invention which comprises phenylephrine hydrochloride and carbinoxamine maleate in a first sustained release layer and codeine phosphate in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Phenylepherine HCl | 75.0 | 185.2 |
| Carbinoxamine Maleate | 8.0 | 19.8 |
| Methocel K4M | 59.0 | 145.7 |
| Silicified Microcrystalline Cellulose | 30.0 | 74.1 |
| Eudragit NE | 15.0 | 37.0 |
| Magnesium Stearate | 3.0 | 7.4 |

Layer 2 (Sustained release)

| | | |
|---|---|---|
| Codeine Phosphate | 30.0 | 74.1 |
| Microcrystalline Cellulose (PH 102) | 45.0 | 111.1 |
| Eudragit NE | 15.0 | 37.0 |
| Methocel K4M Premium | 100.0 | 246.9 |
| Stearic Acid | 20.0 | 49.4 |
| Magnesium Stearate | 5.0 | 12.3 |
| Total | 405.0 | 1000.0 |

Procedure:

(a) Sustained release layer #1: Mix the phenylephrine HCl, carbinoxamine maleate, Methocel®K4M and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 190 mgs and layer #2 is 215 mgs. Capsules may be manufactured by filling the same proportions into capsules.

### EXAMPLE 9

### Bi-Layered Tablet (Wet Granulation)

A bi-layered tablet in accordance with the present invention which comprises pseudoephedrine hydrochloride and chlorpheniramine maleate in a first sustained release layer and codeine phosphate in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Pseudoephedrine HCl | 120.0 | 253.2 |
| Chlorpheniramine Maleate | 12.0 | 25.3 |
| Methocel K4M | 70.0 | 147.7 |
| Silicified Microcrystalline Cellulose | 35.0 | 73.9 |
| Eudragit NE | 20.0 | 42.2 |
| Magnesium Stearate | 3.0 | 6.3 |

Layer 2 (Sustained release)

| | | |
|---|---|---|
| Codeine Phosphate | 30.0 | 63.3 |
| Microcrystalline Cellulose (PH 102) | 45.0 | 95.0 |
| Eudragit NE | 15.0 | 31.7 |
| Methocel K4M Premium | 100.0 | 211.0 |
| Stearic Acid | 20.0 | 42.2 |
| Magnesium Stearate | 5.0 | 10.6 |
| Total | 475.0 | 1000.0 |

Procedure:

(a) Sustained release layer #1: Mix the pseudoephedrine HCl, chlorpheniramine maleate, Methocel®K4M and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 260 mgs and layer #2 is 215 mgs. Capsules may be manufactured by filling the same proportions into capsules.

### EXAMPLE 10

### Bi-Layered Tablet (Wet Granulation)

A bi-layered tablet in accordance with the present invention which comprises carbinoxamine maleate in a first sustained release layer and codeine phosphate in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Carbinoxamine Maleate | 8.0 | 19.3 |
| Lactose Monohydrate | 61.0 | 147.0 |
| Methocel K4M | 70.0 | 168.7 |
| Silicified Microcrystalline Cellulose | 39.0 | 94.0 |
| Eudragit NE | 20.0 | 48.2 |
| Magnesium Stearate | 2.0 | 4.82 |

Layer 2 (Sustained release)

| | | |
|---|---|---|
| Codeine Phosphate | 30.0 | 72.3 |
| Microcrystalline Cellulose (PH 102) | 45.0 | 108.5 |
| Eudragit NE | 15.0 | 36.2 |
| Methocel K4M Premium | 100.0 | 241.0 |
| Stearic Acid | 20.0 | 48.2 |
| Magnesium Stearate | 5.0 | 12.1 |
| Total | 415.0 | 1000.0 |

Procedure:

(a) Sustained release layer #1: Mix the carbinoxamine maleate, Methocel®K4M, lactose monohydrate and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 200 mgs and layer #2 is 215 mgs. Capsules may be manufactured by filling the same proportions into capsules.

### EXAMPLE 11

### Bi-Layered Tablet (Direct Compression)

A bi-layered tablet in accordance with the present invention which comprises promethazine hydrochloride (longer half-life drug) in an immediate release layer and codeine phosphate (shorter half-life drug) in a sustained release layer is illustrated as follows:

| | | |
|---|---|---|
| | Weight/tablet | Weight/1 kg batch |
| Ingredients | (mg) | (in grams) |

Layer 1 (Immediate release)

| | | |
|---|---|---|
| Promethazine HCl | 25 | 45.5 |
| Silicified Microcrystalline | 114.0 | 207.5 |
| Cellulose | | |
| Sodium Starch Glycolate | 10.0 | 18.2 |
| Magnesium Stearate | 1.0 | 1.8 |

Layer 2 (Sustained release)

| | | |
|---|---|---|
| Codeine Phosphate | 60.0 | 109.2 |
| Lactose Monohydrate | 50.0 | 91.0 |
| Dicalcium Phosphate | 50.0 | 91.0 |
| Kollidon SR | 220.0 | 400.4 |
| Stearic acid | 15.0 | 27.3 |
| Magnesium Stearate | 5.0 | 9.1 |
| Total | 550.0 | 1000.0 |

Procedure:

(a) Immediate release layer #1: Screen all ingredients through a USP sieve size # 30. Blend the promethazine hydrochloride, microcrystalline cellulose and sodium starch glycolate for 20 minutes. Add magnesium stearate to the above blend and mix for an additional time of three minutes.

(b) Sustained release layer #2: Blend the codeine phosphate, lactose monohydrate, dicalcium phosphate and Kollidon® SR for 20 minutes. Add stearic acid and magnesium stearate to the above blend and mix for an additional time of three minutes.

Manufacture bi-layered tablets using a rotary bi-layer tablet press where in each tablet the immediate release layer #1 is 150 mgs and the sustained release layer #2 is 400 mgs. Capsules may be manufactured by filling the same proportions into capsules.

### EXAMPLE 12

### Bi-Layered Tablet (Wet Granulation)

A bi-layered tablet in accordance with the present invention which comprises pseudoephedrine tannate and chlorpheniramine tannate in an immediate release layer and codeine phosphate in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Pseudoephedrine Tannate | 60.0 | 85.7 |
| Chlorpheniramine Tannate | 8.0 | 11.4 |
| Silicified Microcrystalline Cellulose | 108.0 | 154.3 |
| Povidone | 3.0 | 4.3 |
| Croscarmellose Sodium | 10.0 | 14.3 |
| Magnesium Stearate | 1.0 | 1.4 |

Layer 2 (Sustained release)

| | | |
|---|---|---|
| Codeine Phosphate | 30.0 | 42.9 |
| Microcrystalline Cellulose (PH 102) | 30.0 | 42.9 |
| Lactose Monohydrate | 100.0 | 142.9 |
| Dicalcium Phosphate | 100.0 | 142.9 |
| Povidone | 15.0 | 21.4 |
| Methocel K4M Premium | 210.0 | 300.0 |
| Stearic Acid | 20.0 | 28.6 |
| Magnesium Stearate | 5.0 | 7.1 |
| Total | 700.0 | 1000.0 |

Procedure:

(a) Immediate release layer #1: Mix the pseudoephedrine tannate, chlorpheniramine tannate, silicified microcrystalline cellulose and croscarmellose sodium, in a high shear mixer/granulator for 10 minutes. Granulate the above blend using a 30% povidone solution (3.0 gms povidone in 10.0 gms purified water). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Mix the codeine phosphate, microcrystalline cellulose PH 102, lactose monohydrate, dicalcium phosphate, Methocel K4M Premium and stearic acid in a high shear mixer/granulator for 10 minutes. Granulate the above blend using a 30% povidone solution (15.0 gms povidone in 50.0 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 190 mgs and layer #2 is 510 mgs. Capsules may be manufactured by filling the same proportions into capsules.

### EXAMPLE 13

### Bi-Layered Tablet (Wet Granulation)

A bi-layered tablet in accordance with the present invention which comprises promethazine hydrochloride in an immediate release layer and codeine phosphate and phenylephrine hydrochloride in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Promethazine HCl | 25 | 55.5 |
| Silicified Microcrystalline Cellulose | 86.0 | 190.0 |
| Povidone | 3.0 | 6.7 |
| Croscarmellose Sodium | 10.0 | 22.2 |
| Magnesium Stearate | 1.0 | 2.2 |

Layer 2 (Sustained release)

| | | |
|---|---|---|
| Codeine Phosphate | 30.0 | 66.6 |
| Phenylepherine HCl | 75.0 | 166.5 |
| Microcrystalline Cellulose (PH 102) | 30.0 | 66.6 |
| Dicalcium Phosphate | 30.0 | 66.6 |
| Povidone | 15.0 | 33.3 |
| Methocel K4M Premium | 120.0 | 266.4 |
| Stearic Acid | 20.0 | 44.4 |
| Magnesium Stearate | 5.0 | 11.1 |
| Total | 450.0 | 1000.0 |

Procedure:

(a) Immediate release layer #1: Mix the promethazine hydrochloride, silicified microcrystalline cellulose and croscarmellose sodium, in a high shear mixer/granulator for 10 minutes. Granulate the above blend using a 30% povidone solution (3.0 gms povidone in 10.0 gms purified water). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Mix the codeine phosphate, phenylephrine HCl, microcrystalline cellulose PH 102, dicalcium phosphate, Methocel K4M Premium and stearic acid in a high shear mixer/granulator for 10 minutes. Granulate the above blend using a 30% povidone solution (15.0 gms povidone in 50.0 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 125 mgs and layer #2 is 325 mgs. Capsules may be manufactured by filling the same proportions into capsules.

### EXAMPLE 14

### Bi-Layered Tablet (Direct Compression)

A bi-layered tablet in accordance with the present invention which comprises guaifenesin in a first sustained release layer and codeine phosphate in a second sustained release layer is illustrated as follows:

| | | |
|---|---|---|
| | Weight/tablet | Weight/1 kg batch |
| Ingredients | (mg) | (in grams) |

Layer 1 (Sustained release)

| | | |
|---|---|---|
| Guaifenesin | 600.0 | 499.8 |
| Methocel K15M | 200.0 | 166.6 |
| Silicified Microcrystalline | 72 | 60.0 |
| Cellulose | | |
| Magnesium Stearate | 8.0 | 6.7 |

Layer 2 (Sustained release)

| | | |
|---|---|---|
| Codeine Phosphate | 60.0 | 50.0 |
| Lactose Monohydrate | 35.0 | 29.2 |
| Dicalcium Phosphate | 35.0 | 29.2 |
| Kollidon SR | 170.0 | 141.6 |
| Stearic acid | 15.0 | 12.5 |
| Magnesium Stearate | 5.0 | 4.2 |
| Total | 1200.0 | 1000.0 |

Procedure:

(a) Sustained release layer #1: Screen all ingredients through a USP sieve size # 30. Blend the guaifenesin, Methocel® K15M and silicified microcrystalline cellulose for 25 minutes. Add magnesium stearate to the above blend and mix for an additional time of three minutes.

(b) Sustained release layer #2: Blend the codeine phosphate, lactose monohydrate, dicalcium phosphate and Kollidon® SR for 20 minutes. Add stearic acid and magnesium stearate to the above blend and mix for an additional time of three minutes.

Manufacture bi-layered tablets using a rotary bi-layer tablet press where in each tablet layer #1 is 880 mgs and layer #2 is 320 mgs. Capsules may be manufactured by filling the same proportions into capsules.

### EXAMPLE 15

### Bi-Layered Tablet (Wet Granulation)

A bi-layered tablet in accordance with the present invention which comprises guaifenesin in a first sustained release layer and codeine phosphate and phenylephrine hydrochloride in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Guaifenesin | 600.0 | 558.0 |
| Methocel K15M | 100.0 | 93.0 |
| Silicified Microcrystalline Cellulose | 50 | 46.5 |
| Eudragit NE | 42 | 39.1 |
| Magnesium Stearate | 8.0 | 7.4 |

Layer 2 (Sustained release)

| | | |
|---|---|---|
| Codeine Phosphate | 30.0 | 27.9 |
| Phenylepherine HCl | 60.0 | 55.8 |
| Microcrystalline Cellulose (PH 102) | 45.0 | 41.9 |
| Eudragit NE | 15.0 | 14.0 |
| Methocel K4M Premium | 100.0 | 93.0 |
| Stearic Acid | 20.0 | 18.6 |
| Magnesium Stearate | 5.0 | 4.7 |
| Total | 1075.0 | 1000.0 |

Procedure:

(a) Sustained release layer #1: Mix the guaifenesin, Methocel®K15M and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, phenylephrine HCl, microcrystalline cellulose PH 102, dicalcium phosphate and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 800 mgs and layer #2 is 275 mgs. Capsules may be manufactured by filling the same proportions into capsules.

### EXAMPLE 16

### Bi-Layered Tablet (Wet Granulation)

A bi-layered tablet in accordance with the present invention which comprises guaifenesin in a first sustained release layer and codeine phosphate and phenylephrine hydrochloride in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Guaifenesin | 1000.0 | 635.0 |
| Methocel K15M | 200.0 | 127.0 |
| Silicified Microcrystalline Cellulose | 40.0 | 25.4 |
| Eudragit NE | 50.0 | 31.8 |
| Magnesium Stearate | 10.0 | 6.4 |

Layer 2 (Sustained release)

| | | |
|---|---|---|
| Codeine Phosphate | 30.0 | 19.1 |
| Phenylepherine HCl | 60.0 | 38.1 |
| Microcrystalline Cellulose (PH 102) | 45.0 | 28.6 |
| Eudragit NE | 15.0 | 9.5 |
| Methocel K4M Premium | 100.0 | 63.5 |
| Stearic Acid | 20.0 | 12.7 |
| Magnesium Stearate | 5.0 | 3.2 |
| Total | 1575.0 | 1000.0 |

Procedure:

(a) Sustained release layer #1: Mix the guaifenesin, Methocel®K15M and silicified microcrystalline cellulose in a high shear mixer/granulator for 10 minutes. Granulate the above blend using a Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the codeine phosphate, phenylephrine HCl, microcrystalline cellulose PH 102, dicalcium phosphate and stearic acid in a high shear mixer/granulator for 10 minutes. Granulate the above blend using a Eudragit®O NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 1300 mgs and layer #2 is 275 mgs. Capsules may be manufactured by filling the same proportions into capsules.

### EXAMPLE 17

### Bi-Layered Tablet (Direct Compression)

A bi-layered tablet in accordance with the present invention which comprises codeine phosphate in a first sustained release layer and phenylephrine hydrochloride and chlorpheniramine maleate in a second sustained release layer is illustrated as follows:

| | | |
|---|---|---|
| | Weight/tablet | Weight/1 kg batch |
| Ingredients | (mg) | (in grams) |

Layer 1 (Sustained release)

| | | |
|---|---|---|
| Codeine Phosphate | 30 | 54.5 |
| Methocel K4M | 50 | 90.9 |
| Silicified Microcrystalline | 100.0 | 181.8 |
| Cellulose | | |
| Sodium Starch Glycolate | 10.0 | 18.2 |
| Magnesium Stearate | 1.0 | 1.8 |

Layer 2 (Sustained release)

| | | |
|---|---|---|
| Phenylepherine HCl | 60 | 109 |
| Chlorpheniramine Maleate | 8.0 | 14.5 |
| Lactose Monohydrate | 50.0 | 90.9 |
| Dicalcium Phosphate | 50.0 | 90.9 |
| Methocel K4M | 181.0 | 329.1 |
| Stearic acid | 15.0 | 27.3 |
| Magnesium Stearate | 5.0 | 9.1 |
| Total | 550.0 | 1000.0 |

Procedure:

(a) Sustained release Layer #1: Screen all ingredients through a USP sieve size # 30. Preblend a portion of the Kollidon SR (145 gms) and all the codeine phosphate for 15 minutes. Add lactose monohydrate (90.9 gms) and dicalcium phosphate (90.9 gms) to the above preblend and mix for an additional 20 minutes. Add stearic acid (27.3 gms) and magnesium stearate (9.1 gms) to the above blend and mix for three minutes.

(b) Sustained release layer #2: Screen all ingredients through a USP sieve size # 30. Preblend a portion of the Kollidon SR (145 gms) and all the chlorpheniramine maleate (14.5 gms) for 15 minutes. Add the remaining Kollidon SR (313.2 gms), phenylephrine hydrochloride (36.4 gms), lactose monohydrate (90.9 gms) and dicalcium phosphate (90.9 gms) to the above preblend and mix for an additional 20 minutes. Add stearic acid (27.3 gms) and magnesium stearate (9.1 gms) to the above blend and mix for three minutes.

Manufacture bi-layered tablets using a rotary bi-layer tablet press where in each tablet the immediate release layer is 150 mgs and the sustained release layer is 400 mgs.

### EXAMPLE 18

### Bi-Layered Tablet (Direct Compression)

By using the process described in Example 17, a bi-layered tablet which contains codeine phosphate in an immediate release layer and codeine phosphate, phenylephrine hydrochloride and chlorpheniramine maleate in a sustained release layer may be manufactured by using direct compression:

| | |
|---|---|
| | Weight/tablet |
| Ingredients | (mgs) |

Layer 1 (Immediate Release)

| | |
|---|---|
| Codeine Phosphate | 10 |
| Silicified Microcrystalline Cellulose | 133.5 |
| Sodium Starch Glycolate | 15 |
| Magnesium Stearate | 1.5 |

Layer 2 (Sustained Release)

| | |
|---|---|
| Codeine Phosphate | 40 |
| Phenylepherine HCl | 50 |
| Chlorpheniramine Maleate | 8 |
| Lactose Monohydrate | 50 |
| Dicalcium Phosphate | 50 |
| Kollidon SR | 252 |
| Stearic Acid | 15 |
| Magnesium Stearate | 5 |
| Total | 620 |

### EXAMPLE 19

### Bi-Layered Tablet (Wet Granulation)

A bi-layered tablet in accordance with the present invention which comprises codeine phosphate in an immediate release layer and codeine phosphate, pseudoephedrine hydrochloride and chlorpheniramine maleate in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Codeine Phosphate | 10.0 | 11.9 |
| Silicified Microcrystalline Cellulose | 111.0 | 158.6 |
| Povidone | 3.0 | 4.3 |
| Croscarmellose Sodium | 10.0 | 14.3 |
| Magnesium Stearate | 1.0 | 1.4 |

Layer 2 (Sustained release)

| | | |
|---|---|---|
| Codeine Phosphate | 30 | 35.7 |
| Pseudoephedrine HCl | 60.0 | 85.7 |
| Chlorpheniramine Maleate | 8.0 | 11.4 |
| Microcrystalline Cellulose (PH 102) | 30.0 | 42.9 |
| Lactose Monohydrate | 100.0 | 142.9 |
| Dicalcium Phosphate | 100.0 | 142.9 |
| Povidone | 15.0 | 21.4 |
| Methocel K4M Premium | 212.0 | 302.9 |
| Stearic Acid | 20.0 | 28.6 |
| Magnesium Stearate | 5.0 | 7.1 |
| Total | 700.0 | 1012.0 |

Procedure:

(a) Immediate release layer #1: Screen all ingredients through a USP sieve size # 30. Blend the codeine phosphate (11.9 grams), silicified microcrystalline cellulose (158.6 grams), and croscarmellose sodium in a high shear mixer/granulator for 10 minutes. Granulate the above blend using a 30% povidone solution (4.3 gms povidone in 14.3 gms purified water). Dry the granulation until the LOD is less than 2.0%. Screen granules through a USP sieve size # 14. Add granules and the prescreened magnesium stearate (1.4 gms) to the above blend and mix for 3 minutes.

(b) Sustained release layer #2: Screen all ingredients through a USP sieve size # 30. Blend the pseudoephedrine hydrochloride (87.5 gms), chlorpheniramine maleate (11.4 gms), codeine phosphate (37.5 gms), microcrystalline cellulose PH 102 (42.9 gms), lactose monohydrate (142.9 gms), dicalcium phosphate (142.9 gms), Methocel K4M Premium (302.9 gms) and stearic acid (28.6 gms) in a high shear mixer/granulator for 10 minutes. Granulate the above blend using a 30% povidone solution (21.4 gms povidone in 71.3 gms purified water). Dry the granulation until the LOD is less than 2.0%. Screen granules through a USP sieve size # 14. Add granules and the prescreened magnesium stearate (7.1 gms) to the above blend and mix for 3 minutes.

Manufacture bi-layered tablets using a rotary bi-layer tablet press where in each tablet the immediate release layer is 150 mgs and the sustained release layer is 550 mgs.

### EXAMPLE 20

### Bi-Layered Tablet (Wet Granulation)

By using the process described in Example 19, a bi-layered tablet containing codeine phosphate in an immediate release layer and pseudoephedrine hydrochloride and chlorpheniramine maleate in a sustained release layer may be manufactured by using wet granulation:

| | |
|---|---|
| | Weight/tablet |
| Ingredients | (mgs) |

Layer 1 (Immediate Release)

| | |
|---|---|
| Codeine Phosphate | 30 |
| Silicified Microcrystalline cellulose | 129.5 |
| Povidone | 4 |
| Croscarmellose sodium | 15 |
| Magnesium Stearate | 1.5 |

Layer 2 (Sustained Release)

| | |
|---|---|
| Pseudoephedrine HCl | 60 |
| Chlorpheniramine Maleate | 8 |
| Microcrystalline Cellulose 102 | 30 |
| Lactose Monohydrate | 100 |
| Dicalcium Phosphate | 100 |
| Povidone | 15 |
| Hydroxypropylmethylcellulose | 212 |
| Stearic Acid | 20 |
| Magnesium Stearate | 5 |
| Total | 750 |

The above examples 6-20 illustrate how to manufacture a bi-layered tablet containing an antitussive, i.e., codeine phosphate in (at least) a first layer and an antihistamine and/or a decongestant and/or an expectorant in (at least) a second layer. Non-limiting examples of possible active ingredients (in addition to the antitussive morphine derivative) in an exemplary range as described in the above Table 1 can be employed depending on the specific therapeutic effect desired.

### EXAMPLE 21

### Extended Release Suspension (Gel)

An extended release suspension (in the form of a gel) in accordance with the present invention which contains a codeine phosphate ion-exchange complex and promethazine hydrochloride is illustrated as follows (note that the codeine phosphate is used in a controlled release form since it has a shorter half-life than the promethazine hydrochloride):

| Ingredients | Amount/5 ml | |
|---|---|---|
| Codeine Phosphate Ion-Exchange | Equivalent to 30 mgs of Codeine | |
| Complex | Phosphate | |
| Promethazine HCl | 25 | mgs |
| Eudragit ® L 100 | 0.2 to 2.8 | grams |
| Glycerin | 315 | mgs |
| Polysorbate 80 | 1.5 | mgs |
| Carbomer (e.g., Carbopol ® 974) | 37.5 | mgs |
| Methyl Paraben | 9 | mgs |
| Propyl Paraben | 1 | mgs |
| Saccharin Sodium cryst., USP | 0.1 | mg |
| Artificial Grape Flavor | 5 | mgs |
| FD&C Red # 40 Dye | 0.5 | mgs |

| | |
|---|---|
| Sodium Hydroxide | q.s. |
| Water | q.s |

Procedure:

(1) Add the appropriate amount of sodium polystyrene sulphonate USP (e.g. Amberlite® IRP 69) to a codeine phosphate solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eudragit® L 100, Kollidon® MAE, Aquacoat® cPD) and dry the granules.

(5) Mill the granules, if needed.

(6) To an appropriate amount of water add the following ingredients and dissolve: promethazine hydrochloride, Carbomer (e.g., Carbopol® 974), glycerin, polysorbate 80, methyl paraben, propyl paraben, artificial grape flavor, FD&C red # 40 dye.

(7) Add milled granules.

(8) Add water to 95% of final volume.

(9) Agitate at suitable rate to avoid settling of the suspension and maintain a homogeneous product mixture.

(10) Neutralize the solution to form a gel using a IN sodium hydroxide solution. Add water to make final volume.

(11) Fill in suitable containers ensuring that the product is homogeneous throughout the filling operation.

### EXAMPLE 22

### Extended Release Suspension (Liquid)

An extended release suspension (in the form of a liquid) in accordance with the present invention which contains a codeine phosphate ion-exchange complex and promethazine hydrochloride is illustrated as follows:

| Ingredients | Amount/5 ml | |
|---|---|---|
| Codeine Phosphate Ion-Exchange Complex | Equivalent to 45 mgs of | |
| | Codeine Phosphate | |
| Promethazine HCl | 25 | mgs |
| Eudragit ® L 100 | 0.2 to 2.8 | grams |
| Silica, colloidal anhydrous, NF | 100 | mgs |
| Glycerin | 740 | mgs |
| Xylitol, NF | 800 | mgs |
| Sodium Citrate, USP | 100 | mgs |
| Saccharin Sodium cryst., USP, | 0.1 | mg |
| Sodium Benzoate | 7.5 | mgs |
| Citric Acid Monohydrate, USP | 8.0 | mgs |
| Artificial Grape Flavor | 5 | mgs |
| FD&C Red # 40 Dye | 0.5 | mgs |

| | |
|---|---|
| Water | q.s |

Manufacturing Process for 1000 L Batch:

Add the appropriate amount of sodium polystyrene sulphonate USP (e.g. Amberlite® IRP 69) to a codeine phosphate solution. Stir the mix for 12 hrs to allow complete drug/resin complex formation. Separate and dry the insoluble drug/resin complex. Granulate the drug/resin complex with a delayed release/enteric polymer (e.g. Eudragit® L 100, Kollidon® MAE, Aquacoat® CPD) and dry the granules. Mill the granules, if needed.

In a suitably sized stainless steel vessel, dissolve saccharin sodium, sodium benzoate, citric acid, and sodium citrate in approximately 50 L of warm (about 45° C.), purified water. In another large stainless steel drum mix the silica, codeine phosphate ion-exchange complex, and promethazine hydrochloride until a uniform and consistent mixture is obtained. In a separate 1000 L stainless steel tank equipped with a suitably sized homogenizer/disperser add about 100 L of purified water. With the homogenizer on, add the silica mixture containing codeine phosphate ion-exchange complex, and promethazine hydrochloride. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes.

### EXAMPLE 23

### Extended Release Suspension (Liquid)

An extended release suspension (in the form of a liquid) in accordance with the present invention which contains a codeine phosphate ion-exchange complex, pseudoephedrine tannate and chlorpheniramine tannate is illustrated as follows:

| Ingredients | Amount/5 ml | |
|---|---|---|
| Codeine Phosphate Ion-Exchange Complex | Equivalent to 45 mgs of | |
| | Codeine Phosphate | |
| Pseudoephedrine Tannate | 75.0 | |
| Chlorpheniramine Tannate | 4.5 | |
| Eudragit ® L 100 | 0.2 to 2.8 | grams |
| Silica, colloidal anhydrous, NF | 100 | mgs |
| Glycerin | 740 | mgs |
| Xylitol, NF | 800 | mgs |
| Sodium Citrate, USP | 100 | mgs |
| Saccharin Sodium cryst., USP, | 0.1 | mg |
| Sodium Benzoate | 7.5 | mgs |
| Citric Acid Monohydrate, USP | 8.0 | mgs |
| Artificial Grape Flavor | 5 | mgs |
| FD&C Red # 40 Dye | 0.5 | mgs |

| | |
|---|---|
| Water | q.s |

Manufacturing Process for 1000 kg Batch:

Add the appropriate amount of sodium polystyrene sulphonate USP (e.g. Amberlite® IRP 69) to a codeine phosphate solution. Stir the mix for 12 hrs to allow complete drug/resin complex formation. Separate and dry the insoluble drug/resin complex. Granulate the drug/resin complex with a delayed release/enteric polymer (e.g. Eudragit® L 100, Kollidon® MAE, Aquacoat® CPD) and dry the granules. Mill the granules, if needed.

In a suitably sized stainless steel vessel, dissolve saccharin sodium, sodium benzoate, citric acid, and sodium citrate in approximately 50 L of warm (about 45° C.), purified water. In another large stainless steel drum mix the silica, codeine phosphate ion-exchange complex, pseudoephedrine tannate, and the chlorpheniramine tannate until a uniform and consistent mixture is obtained. In a separate 1000 L stainless steel tank equipped with a suitably sized homogenizer/disperser add about 100 L of purified water. With the homogenizer on, add the silica mixture containing codeine phosphate ion-exchange complex, pseudoephedrine tannate, and the chlorpheniramine tannate. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes.

### EXAMPLE 24

### Extended Release Suspension

An extended release suspension which contains a hydrocodone bitartrate ion-exchange complex, a dexchlorpheniramine maleate ion-exchange complex and a phenylephrine hydrochloride ion-exchange complex is illustrated as follows:

| Ingredients | Amount/5 ml | |
|---|---|---|
| Hydrocodone Bitartrate Ion-Exchange Complex | Equivalent to 8 mgs of | |
| | Hydrocodonee bitartarate | |
| Dexchlorpheniramine Maleate Ion-Exchange | Equivalent to 4 mgs of | |
| Complex | Dexchlorpheniramine | |
| | Maleate | |
| Phenylepherine HCl Ion-Exchange Complex | Equivalent to 10 mgs of | |
| | Phenylepherine HCl | |
| Eudragit ® L 100 | 0.2 to 2.8 | grams |
| Glycerin | 315 | mgs |
| Polysorbate 80 | 1.5 | mgs |
| Carbomer (e.g., Carbopol ® 974) | 15 | mgs |
| Methyl Paraben | 9 | mgs |
| Propyl Paraben | 1 | mgs |
| Artificial Grape Flavor | 5 | mgs |
| FD&C Red # 40 Dye | 0.5 | mgs |

| | |
|---|---|
| Water | q.s |

The formula described above serves as a non-limiting example. Any active drug which is in the form of a salt, such as codeine, or dihydrocodeine, or hydrocodone can be incorporated as an ion-exchange resin complex.

Procedure:

(1) Add the appropriate amount of sodium polystyrene sulphonate USP (e.g. Amberlite® IRP 69) to a codeine phosphate, dexchlorpheniramine maleate and phenylephrine HCl solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eugragit® L 100, Kollidon® MAE, Aquacoa®t cPD) and dry the granules.

(5) Mill the granules, if needed.

(6) To an appropriate amount of water add the following ingredients and dissolve: Carbomer (e.g., Carbopol® 974), glycerin, polysorbate 80, methyl paraben, propyl paraben, artificial grape flavor, FD&C red # 40 dye.

(7) Add milled granules.

(8) Add water to make up to a final volume.

(9) Agitate at suitable rate to avoid settling of the suspension and maintain a homogeneous product mixture.

(10) Fill in suitable containers ensuring that the product is homogeneous throughout the filling operation.

### EXAMPLE 25

### Suspension Formula

A suspension formula which comprises codeine phosphate and phenylephrine tannate is illustrated as follows:

| | g/100 mL = | | kg/batch = | |
|---|---|---|---|---|
| Ingredients | 120 g | | 1000 kg | |
| Codeine Phosphate | 0.500 | | 4.167 | |
| Phenylephrine Tannate | 0.800 | | 6.667 | |
| Silica, colloidal anhydrous, NF | 1.73 | | 14.417 | |
| Hydroxyethylcellulose, NF | 0.05 | | 0.417 | |
| Sorbitol Solution 70% (non-crystallizing), | 34.00 | | 283.333 | |
| NF | | | | |
| Glycerol | 14.75 | | 122.917 | |
| Xylitol, NF | 16.00 | | 133.333 | |
| Sodium Citrate, USP | 2.00 | | 16.667 | |
| Saccharin Sodium cryst., USP, | 0.01 | | 0.083 | |
| Sodium Benzoate, NF | 0.15 | | 1.250 | |
| Citric Acid Monohydrate, USP | 0.16 | | 1.333 | |
| Strawberry Flavor | 0.15 | | 1.250 | |
| Banana Flavor | 0.15 | | 1.250 | |
| Purified Water | 49.55 | | 412.917 | |
| Total Amount | 120.000 | g | 1000.000 | kg |

Manufacturing Process for 1000 kg Batch:

In a suitably sized stainless steel vessel, dissolve saccharin sodium, sodium benzoate, citric acid, and sodium citrate in approximately 50 L of warm (about 45° C.), purified water. In another large stainless steel drum mix the silica, codeine phosphate, and micronized phenylephrine tannate until a uniform and consistent mixture is obtained. In a separate 1000 L stainless steel tank equipped with a suitably sized homogenizer/disperser add about 100 L of purified water. With the homogenizer on, add the silica mixture containing phenylephrine tannate and codeine phosphate. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes. Filter product through a 10 micron filter and fill in appropriately sized containers.

To make products with other agents such as antihistamines, decongestants, or expectorants, one or more combinations of each of the ingredients in a range as described in Table 1 below can be made depending on the specific therapeutic effect desired.

### EXAMPLE 26

### Liquid Formula

A liquid dosage form which comprises codeine phosphate and phenylephrine hydrochloride is illustrated as follows:

| Ingredients | Per 5 mL | | Per 425 L | |
|---|---|---|---|---|
| Codeine Phosphate USP | 30 | mg | 2.550 | kg |
| Phenylepherine Hydrochloride USP | 10.0 | mg | 0.850 | kg |
| Methyl Paraben USP | 9.0 | mg | 0.765 | kg |
| Propyl Paraben USP | 1.0 | mg | 0.085 | kg |
| Propylene Glycol USP | 259 | mg | 22.016 | kg |
| Saccharin Sodium USP | 3.18 | mg | 0.270 | kg |
| Citric Acid USP | 5.0 | mg | 0.425 | kg |
| Strawberry Flavor | 10 | mg | 0.850 | kg |
| Banana Flavor | 10 | mg | 0.850 | kg |
| Sorbitol Solution 70% USP | 3212.5 | mg | 273.1 | kg |
| Purified Water, as required to q.s. to | 5.0 | mL | 425 | L |

Manufacturing Process for 425 L Batch Size:

In a suitably sized stainless steel vessel, dissolve methyl paraben and propyl paraben in approximately 50 L of warm (about 45° C.), purified water. Add about half of the propylene glycol and mix for about 1 hr. In a separate 1000 L stainless steel tank equipped with a suitably sized agitator, add about 50 L of purified water. With the agitator on, add phenylephrine hydrochloride, codeine phosphate, saccharin sodium and citric acid and dissolve. Add the previously prepared paraben/propylene glycol solution to the 1000 L tank. Rinse the first vessel with about 2 L of water and transfer the rinsate to the 1000 L tank. Add the remaining propylene glycol to a suitably sized stainless steel vessel and dissolve the strawberry and banana flavors. Transfer this to the 1000 L tank. Rinse the container with 2 L of purified water and transfer to the 1000 L tank. With the agitator on, add the sorbitol solution 70% to the 1000 L tank. In a suitably sized stainless steel vessel, dissolve the codeine phosphate in about 5 L of purified water and transfer to the 1000 L tank. Rinse the container with about 2 L of purified water and transfer to the 1000 L tank. Stop the agitator and let the solution stand for 15 minutes. QS to 425 L with purified water. Filter product through a 1 micron filter and fill in appropriately sized containers.

To make products with other antihistamines, decongestants, or expectorants, one or more combinations of each of the ingredients in a range as described in Table 1 above can be made depending on the specific therapeutic effect desired.

### EXAMPLE 27

### Bi-Layered Tablet (Wet Granulation)

A bi-layered tablet in accordance with the present invention which comprises guaifenesin in a first sustained release layer and carbetapentane citrate and pseudoephedrine hydrochloride in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Guaifenesin | 600.0 | 510.6 |
| Methocel K15M | 100.0 | 85.1 |
| Silicified Microcrystalline Cellulose | 50 | 42.6 |
| Eudragit NE | 42 | 35.7 |
| Magnesium Stearate | 8.0 | 6.8 |

Layer 2 (Sustained release)

| | | |
|---|---|---|
| Carbetapentane Citrate | 30.0 | 25.5 |
| Pseudoephedrine HCl | 120.0 | 102.1 |
| Microcrystalline Cellulose (PH 102) | 45.0 | 38.4 |
| Eudragit NE | 15.0 | 12.8 |
| Methocel K4M Premium | 140.0 | 119.1 |
| Stearic Acid | 20.0 | 17.0 |
| Magnesium Stearate | 5.0 | 4.3 |
| Total | 1175.0 | 1000.0 |

Procedure:

(a) Sustained release layer #1: Mix the guaifenesin, Methocel®K15M and silicified microcrystalline cellulose in a high shear mixer/granulator for 10 minutes. Granulate the above blend using the Eudragit® NE (30%). Dry the granulation until the LOD (weight loss on drying) is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size# 30. Mix the carbetapentane citrate, pseudoephedrine HCl, microcrystalline cellulose PH 1 02, and stearic acid in a high shear mixer/granulator for 10 minutes. Granulate the above blend using the Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 800 mgs and layer #2 is 375 mgs. Capsules may be manufactured by filling the same proportions into capsules.

### EXAMPLE 28

### Bi-Layered Tablet (Wet Granulation)

A bi-layered tablet in accordance with the present invention which comprises promethazine hydrochloride in an immediate release layer and carbetapentane citrate and pseudoephedrine hydrochloride in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Promethazine HCl | 25.0 | 37.0 |
| Silicified Microcrystalline Cellulose | 111.0 | 164.3 |
| Povidone | 3.0 | 4.4 |
| Croscarmellose Sodium | 10.0 | 14.8 |
| Magnesium Stearate | 1.0 | 1.5 |

Layer 2 (Sustained release)

| | | |
|---|---|---|
| Carbetapentane Citrate | 30.0 | 44.4 |
| Pseudoephedrine HCl | 120.0 | 177.6 |
| Microcrystalline Cellulose (PH 102) | 30.0 | 44.4 |
| Dicalcium Citrate | 100.0 | 148.0 |
| Povidone | 15.0 | 22.2 |
| Methocel K4M Premium | 205.0 | 304.4 |
| Stearic Acid | 20.0 | 29.6 |
| Magnesium Stearate | 5.0 | 7.4 |
| Total | 675.0 | 1000.0 |

Procedure:

(a) Immediate release layer #1: Mix the promethazine HCl, silicified microcrystalline cellulose and croscarmellose sodium, in a high shear mixer/granulator for 10 minutes. Granulate the above blend using a 30% povidone solution (3.0 gms povidone in 10.0 gms purified water). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Mix the carbetapentane citrate, pseudoephedrine HCl, microcrystalline cellulose PH 102, dicalcium phosphate, Methocel K4M Premium and stearic acid in a high shear mixer/granulator for 10 minutes. Granulate the above blend using a 30% povidone solution (15.0 gms povidone in 50.0 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 150 mgs and layer #2 is 525 mgs. Capsules may be manufactured by filling the same proportions into capsules.

### EXAMPLE 29

### Bi-Layered Tablet (Wet Granulation)

A bi-layered tablet in accordance with the present invention which comprises phenylephrine hydrochloride and carbinoxamine maleate in a first sustained release layer and carbetapentane citrate in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Phenylepherine HCl | 75.0 | 185.2 |
| Carbinoxamine Maleate | 8.0 | 19.8 |
| Methocel K4M | 59.0 | 145.7 |
| Silicified Microcrystalline Cellulose | 30.0 | 74.1 |
| Eudragit NE | 15.0 | 37.0 |
| Magnesium Stearate | 3.0 | 7.4 |

Layer 2 (Sustained release)

| | | |
|---|---|---|
| Carbetapentane Citrate | 30.0 | 74.1 |
| Microcrystalline Cellulose (PH 102) | 45.0 | 111.1 |
| Eudragit NE | 15.0 | 37.0 |
| Methocel K4M Premium | 100.0 | 246.9 |
| Stearic Acid | 20.0 | 49.4 |
| Magnesium Stearate | 5.0 | 12.3 |
| Total | 405.0 | 1000.0 |

Procedure:

(a) Sustained release layer #1: Mix the phenylephrine HCl, carbinoxamine maleate, Methocel®K4M and silicified microcrystalline cellulose in a high shear mixer/granulator for 10 minutes. Granulate the above blend using the Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the carbetapentane citrate, microcrystalline cellulose PH 102, and stearic acid in a high shear mixer/granulator for 10 minutes. Granulate the above blend using the Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 190 mgs and layer #2 is 215 mgs. Capsules may be manufactured by filling the same proportions into capsules.

### EXAMPLE 30

### Bi-Layered Tablet (Wet Granulation)

A bi-layered tablet in accordance with the present invention which comprises pseudoephedrine hydrochloride and chlorpheniramine maleate in a first sustained release layer and carbetapentane citrate in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Pseudoephedrine HCl | 120.0 | 253.2 |
| Chlorpheniramine Maleate | 12.0 | 25.3 |
| Methocel K4M | 70.0 | 146.7 |
| Silicified Microcrystalline Cellulose | 35.0 | 73.9 |
| Eudragit NE | 20.0 | 42.2 |
| Magnesium Stearate | 3.0 | 6.3 |

Layer 2 (Sustained release)

| | | |
|---|---|---|
| Carbetapentane Citrate | 30.0 | 63.4 |
| Microcrystalline Cellulose (PH 102) | 45.0 | 95.0 |
| Eudragit NE | 15.0 | 31.7 |
| Methocel K4M Premium | 100.0 | 209.5 |
| Stearic Acid | 20.0 | 42.2 |
| Magnesium Stearate | 5.0 | 10.6 |
| Total | 475.0 | 1000.0 |

Procedure:

(a) Sustained release layer #1: Mix the pseudoephedrine HCl, chlorpheniramine maleate, Methocel®K4M and silicified microcrystalline cellulose in a high shear mixer/granulator for 10 minutes. Granulate the above blend using the Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the carbetapentane citrate, microcrystalline cellulose PH 102, and stearic acid in a high shear mixer/granulator for 10 minutes. Granulate the above blend using the Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer,tablet press where in each tablet layer #1 is 260 mgs and layer #2 is 215 mgs. Capsules may be manufactured by filling the same proportions into capsules.

### EXAMPLE 31

### Bi-Layered Tablet (Wet Granulation)

A bi-layered tablet in accordance with the present invention which comprises carbinoxamine maleate in a first sustained release layer and carbetapentane citrate in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Carbinoxamine Maleate | 8.0 | 19.3 |
| Lactose Monohydrate | 61.0 | 147.0 |
| Methocel K4M | 70.0 | 168.7 |
| Silicified Microcrystalline Cellulose | 39.0 | 94.0 |
| Eudragit NE | 20.0 | 48.2 |
| Magnesium Stearate | 2.0 | 4.82 |

Layer 2 (Sustained release)

| | | |
|---|---|---|
| Carbetapentane Citrate | 30.0 | 72.4 |
| Microcrystalline Cellulose (PH 102) | 45.0 | 108.5 |
| Eudragit NE | 15.0 | 36.2 |
| Methocel K4M Premium | 100.0 | 241.0 |
| Stearic Acid | 20.0 | 48.2 |
| Magnesium Stearate | 5.0 | 12.1 |
| Total | 415.0 | 1000.0 |

Procedure:

(a) Sustained release layer #1: Mix the carbinoxamine maleate, Methocel®K4M, lactose monohydrate and silicified microcrystalline cellulose in a high shear mixer/granulator for 10 minutes. Granulate the above blend using the Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the carbetapentane citrate, microcrystalline cellulose PH 102, and stearic acid in a high shear mixer/granulator for 10 minutes. Granulate the above blend using the Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 200 mgs and layer #2 is 215 mgs. Capsules may be manufactured by filling the same proportions into capsules.

### EXAMPLE 32

### Bi-Layered Tablet (Direct Compression)

A bi-layered tablet in accordance with the present invention which comprises promethazine hydrochloride (longer half-life drug) in an immediate release layer and carbetapentane citrate (shorter half-life drug) in a sustained release layer is illustrated as follows:

| | | |
|---|---|---|
| | Weight/tablet | Weight/1 kg batch |
| Ingredients | (mg) | (in grams) |

Layer 1 (Immediate release)

| | | |
|---|---|---|
| Promethazine HCl | 25 | 45.5 |
| Silicified Microcrystalline | 114.0 | 207.5 |
| Cellulose | | |
| Sodium Starch Glycolate | 10.0 | 18.2 |
| Magnesium Stearate | 1.0 | 1.8 |

Layer 2 (Sustained release)

| | | |
|---|---|---|
| Carbetapentane Citrate | 60.0 | 109.2 |
| Lactose Monohydrate | 50.0 | 91.0 |
| Dicalcium Citrate | 50.0 | 91.0 |
| Kollidon SR | 220.0 | 399.4 |
| Stearic acid | 15.0 | 27.3 |
| Magnesium Stearate | 5.0 | 9.1 |
| Total | 550.0 | 1000.0 |

Procedure:

(a) Immediate release layer #1: Screen all ingredients through a USP sieve size # 30. Blend the promethazine hydrochloride, microcrystalline cellulose and sodium starch glycolate for 20 minutes. Add magnesium stearate to the above blend and mix for an additional time of three minutes.

(b) Sustained release layer #2: Blend the carbetapentane citrate, lactose monohydrate, dicalcium citrate and Kollidon® SR for 20 minutes. Add stearic acid and magnesium stearate to the above blend and mix for an additional time of three minutes.

Manufacture bi-layered tablets using a rotary bi-layer tablet press where in each tablet the immediate release layer #1 is 150 mgs and the sustained release layer #2 is 400 mgs. Capsules may be manufactured by filling the same proportions into capsules.

### EXAMPLE 33

### Bi-Layered Tablet (Wet Granulation)

A bi-layered tablet in accordance with the present invention which comprises pseudoephedrine tannate and chlorpheniramine tannate in an immediate release layer and carbetapentane tannate in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Pseudoephedrine Tannate | 60.0 | 85.7 |
| Chlorpheniramine Tannate | 8.0 | 11.4 |
| Silicified Microcrystalline Cellulose | 108.0 | 154.3 |
| Povidone | 3.0 | 4.3 |
| Croscarmellose Sodium | 10.0 | 14.3 |
| Magnesium Stearate | 1.0 | 1.4 |

Layer 2 (Sustained release)

| | | |
|---|---|---|
| Carbetapentane Tannate | 30.0 | 42.9 |
| Microcrystalline Cellulose (PH 102) | 30.0 | 42.9 |
| Lactose Monohydrate | 100.0 | 142.9 |
| Dicalcium Citrate | 100.0 | 142.9 |
| Povidone | 15.0 | 21.4 |
| Methocel K4M Premium | 210.0 | 300.0 |
| Stearic Acid | 20.0 | 28.6 |
| Magnesium Stearate | 5.0 | 7.1 |
| Total | 700.0 | 1000.0 |

Procedure:

(a) Immediate release layer #1: Mix the pseudoephedrine tannate, chlorpheniramine tannate, silicified microcrystalline cellulose and croscarmellose sodium, in a high shear mixer/granulator for 10 minutes. Granulate the above blend using a 30% povidone solution (3.0 gms povidone in 10.0 gms purified water). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Mix the carbetapentane tannate, microcrystalline cellulose PH 102, lactose monohydrate, dicalcium phosphate, Methocel K4M Premium and stearic acid in a high shear mixer/granulator for 10 minutes. Granulate the above blend using a 30% povidone solution (15.0 gms povidone in 50.0 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 190 mgs and layer #2 is 510 mgs. Capsules may be manufactured by filling the same proportions into capsules.

### EXAMPLE 34

### Bi-Layered Tablet (Wet Granulation)

A bi-layered tablet in accordance with the present invention which comprises diphenhydramine hydrochloride in an immediate release layer and carbetapentane citrate and phenylephrine hydrochloride in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Diphenydramine HCl | 100 | 181.8 |
| Silicified Microcrystalline Cellulose | 86.0 | 156.4 |
| Povidone | 3.0 | 5.5 |
| Croscarmellose Sodium | 10.0 | 18.2 |
| Magnesium Stearate | 1.0 | 1.8 |

Layer 2 (Sustained release)

| | | |
|---|---|---|
| Carbetapentane Citrate | 60.0 | 109.1 |
| Phenylepherine HCl | 75.0 | 136.4 |
| Microcrystalline Cellulose (PH 102) | 30.0 | 54.5 |
| Dicalcium Citrate | 30.0 | 54.5 |
| Povidone | 15.0 | 27.3 |
| Methocel K4M Premium | 115.0 | 209.1 |
| Stearic Acid | 20.0 | 36.4 |
| Magnesium Stearate | 5.0 | 9.1 |
| Total | 450.0 | 1000.0 |

Procedure:

(a) Immediate release layer #1: Mix the diphenhydramine hydrochloride, silicified microcrystalline cellulose and croscarmellose sodium in a high shear mixer/granulator for 10 minutes. Granulate the above blend using a 30% povidone solution (3.0 gms povidone in 10.0 gms purified water). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Mix the carbetapentane citrate, phenylephrine HCl, microcrystalline cellulose PH 102, dicalcium phosphate, Methocel K4M Premium and stearic acid in a high shear mixer/granulator for 10 minutes. Granulate the above blend using a 30% povidone solution (15.0 gms povidone in 50.0 gms purified water). Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 125 mgs and layer #2 is 325 mgs. Capsules may be manufactured by filling the same proportions into capsules.

### EXAMPLE 35

### Bi-Layered Tablet (Direct Compression)

A bi-layered tablet in accordance with the present invention which comprises guaifenesin in a first sustained release layer and carbetapentane citrate in a second sustained release layer is illustrated as follows:

| | | |
|---|---|---|
| | Weight/tablet | Weight/1 kg batch |
| Ingredients | (mg) | (in grams) |

Layer 1 (Sustained release)

| | | |
|---|---|---|
| Guaifenesin | 600.0 | 499.8 |
| Methocel K15M | 200.0 | 166.6 |
| Silicified Microcrystalline | 72 | 60.0 |
| Cellulose | | |
| Magnesium Stearate | 8.0 | 6.7 |

Layer 2 (Sustained release)

| | | |
|---|---|---|
| Carbetapentane Citrate | 60.0 | 50 |
| Lactose Monohydrate | 35.0 | 29.2 |
| Dicalcium Citrate | 35.0 | 29.2 |
| Kollidon SR | 170.0 | 141.6 |
| Stearic acid | 15.0 | 12.5 |
| Magnesium Stearate | 5.0 | 4.2 |
| Total | 1200.0 | 1000.0 |

Procedure:

(a) Sustained release layer #1: Screen all ingredients through a USP sieve size # 30. Blend the guaifenesin, Methocel® K15M and silicified microcrystalline cellulose for 25 minutes. Add magnesium stearate to the above blend and mix for an additional time of three minutes.

(b) Sustained release layer #2: Blend the carbetapentane citrate, lactose monohydrate, dicalcium citrate and Kollidon® SR for 20 minutes. Add stearic acid and magnesium stearate to the above blend and mix for an additional time of three minutes.

Manufacture bi-layered tablets using a rotary bi-layer tablet press where in each tablet layer #1 is 880 mgs and layer #2 is 320 mgs. Capsules may be manufactured by filling the same proportions into capsules.

### EXAMPLE 36

### Bi-Layered Tablet (Wet Granulation)

A bi-layered tablet in accordance with the present invention which comprises guaifenesin in a first sustained release layer and carbetapentane citrate and phenylephrine hydrochloride in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Guaifenesin | 600.0 | 545.5 |
| Methocel K15M | 100.0 | 90.9 |
| Silicified Microcrystalline Cellulose | 50 | 45.5 |
| Eudragit NE | 42 | 38.2 |
| Magnesium Stearate | 8.0 | 7.3 |

Layer 2 (Sustained release)

| | | |
|---|---|---|
| Carbetapentane Citrate | 60.0 | 54.6 |
| Phenylepherine HCl | 60.0 | 54.6 |
| Microcrystalline Cellulose (PH 102) | 45.0 | 40.9 |
| Eudragit NE | 15.0 | 13.6 |
| Methocel K4M Premium | 100.0 | 90.9 |
| Stearic Acid | 20.0 | 13.6 |
| Magnesium Stearate | 5.0 | 4.5 |
| Total | 1100 | 1000 |

Procedure:

(a) Sustained release layer #1: Mix the guaifenesin, Methocel®K15M and silicified microcrystalline cellulose in a high shear mixer/granulator for 10 minutes. Granulate the above blend using the Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the carbetapentane citrate, phenylephrine HCl, microcrystalline cellulose PH 102, dicalcium phosphate and stearic acid in a high shear mixer/granulator for 10 minutes. Granulate the above blend using the Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 800 mgs and layer #2 is 275 mgs. Capsules may be manufactured by filling the same proportions into capsules.

### EXAMPLE 37

### Bi-Layered Tablet (Wet Granulation)

A bi-layered tablet in accordance with the present invention which comprises guaifenesin in a first sustained release layer and carbetapentane citrate and phenylephrine hydrochloride in a second sustained release layer is illustrated as follows:

| | | |
|---|---|---|
| | Weight/tablet | Weight/1 kg |
| Ingredients | (mgs) | batch (gms) |

Layer 1 (Sustained release)

| | | |
|---|---|---|
| Guaifenesin | 1000.0 | 625.0 |
| Methocel K15M | 200.0 | 208.3 |
| Silicified Microcrystalline Cellulose | 40.0 | 41.7 |
| Eudragit NE | 50.0 | 31.3 |
| Magnesium Stearate | 10.0 | 6.3 |

Layer 2 (Sustained release)

| | | |
|---|---|---|
| Carbetapentane Citrate | 60.0 | 37.5 |
| Phenylepherine HCl | 60.0 | 37.5 |
| Microcrystalline Cellulose (PH 102) | 45.0 | 25 |
| Eudragit NE | 15.0 | 9.4 |
| Methocel K4M Premium | 100.0 | 62.5 |
| Stearic Acid | 20.0 | 12.5 |
| Magnesium Stearate | 5.0 | 3.1 |
| Total | 1600.0 | 1000.0 |

Procedure:

(a) Sustained release layer #1: Mix the guaifenesin, Methocel®K15M and silicified microcrystalline cellulose in a high shear mixer/granulator for 10 minutes. Granulate the above blend using the Eudragit® NE (30%). Dry the granulation until the LOD is less than 2.0%. Screen granules through a USP sieve size # 14. Add the granules and the prescreened magnesium stearate in a V shaped blender and mix for 3 minutes.

(b) Sustained release layer #2: Screen all ingredients through a USP sieve size # 30. Mix the carbetapentane citrate, phenylephrine HCl, microcrystalline cellulose PH 102, dicalcium phosphate and stearic acid in a high shear mixer/granulator for 10 minutes. Granulate the above blend using the Eudragit® NE (30%). Add the Methocel®K4M to the granulator and post mix for 5 minutes. Dry the granulation until the LOD is less than 2.0%. Screen granules through a USP sieve size # 14. Add the granules and the prescreened magnesium stearate in a V shaped blender and mix for 3 minutes.

Manufacture bi-layered tablets using a rotary bi-layer tablet press where in each tablet layer #1 is 1300 mgs and layer #2 is 300 mgs. Capsules may be manufactured by filling the same proportions into capsules.

### EXAMPLE 38

### Bi-Layered Tablet (Direct Compression)

By using the process described in Example 37, a bi-layered tablet which contains carbetapentane citrate in an immediate release layer and carbetapentane citrate, phenylephrine hydrochloride and chlorpheniramine maleate in a sustained release layer may be manufactured by using direct compression:

| | |
|---|---|
| | Weight/tablet |
| Ingredients | (mgs) |

Layer 1 (Immediate Release)

| | |
|---|---|
| Carbetapentane Citrate | 10 |
| Silicified Microcrystalline Cellulose | 133.5 |
| Sodium Starch Glycolate | 15 |
| Magnesium Stearate | 1.5 |

Layer 2 (Sustained Release)

| | |
|---|---|
| Carbetapentane Citrate | 40 |
| Phenylepherine HCl | 50 |
| Chlorpheniramine Maleate | 8 |
| Lactose Monohydrate | 50 |
| Dicalcium Citrate | 50 |
| Kollidon SR | 252 |
| Stearic Acid | 15 |
| Magnesium Stearate | 5 |
| Total | 630 |

### EXAMPLE 39

### Bi-Layered Tablet (Wet Granulation)

A bi-layered tablet in accordance with the present invention which comprises carbetapentane citrate in an immediate release layer and carbetapentane citrate, pseudoephedrine hydrochloride and chlorpheniramine maleate in a sustained release layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Carbetapentane Citrate | 10.0 | 13.8 |
| Silicified Microcrystalline Cellulose | 111.0 | 153.1 |
| Povidone | 3.0 | 4.1 |
| Croscarmellose Sodium | 10.0 | 13.8 |
| Magnesium Stearate | 1.0 | 1.4 |

Layer 2 (Sustained release)

| | | |
|---|---|---|
| Carbetapentane Citrate | 40 | 55.2 |
| Pseudoephedrine HCl | 60.0 | 82.8 |
| Chlorpheniramine Maleate | 8.0 | 11 |
| Microcrystalline Cellulose (PH 102) | 30.0 | 41.4 |
| Lactose Monohydrate | 100.0 | 137.9 |
| Dicalcium Citrate | 100.0 | 137.9 |
| Povidone | 15.0 | 20.7 |
| Methocel K4M Premium | 212.0 | 292.4 |
| Stearic Acid | 20.0 | 27.6 |
| Magnesium Stearate | 5.0 | 6.9 |
| Total | 725.0 | 1000.0 |

Procedure:

(a) Immediate release layer #1: Screen all ingredients through a USP sieve size # 30. Blend the carbetapentane citrate, silicified microcrystalline cellulose, and croscarmellose sodium in a high shear mixer/granulator for 10 minutes. Granulate the above blend using a 30% povidone solution (4.1 gms povidone in 13.7 gms of solution). Dry the granulation until the LOD is less than 2.0%. Screen granules through a USP sieve size # 14. Add granules and the prescreened magnesium stearate (1.4 gms) to the above blend and mix for 3 minutes.

(b) Sustained release layer #2: Screen all ingredients through a USP sieve size # 30. Blend the pseudoephedrine hydrochloride (87.5 gms), chlorpheniramine maleate, carbetapentane citrate, microcrystalline cellulose PH 102, lactose monohydrate, dicalcium citrate, Methocel K4M Premium and stearic acid in a high shear mixer/granulator for 10 minutes. Granulate the above blend using a 30% povidone solution (20.7 gms povidone in 69 gms of solution). Dry the granulation until the LOD is less than 2.0%. Screen granules through a USP sieve size # 14. Add granules and the prescreened magnesium stearate (6.9 gms) to the above blend and mix for 3 minutes.

Manufacture bi-layered tablets using a rotary bi-layer tablet press where in each tablet the immediate release layer is 135 mgs and the sustained release layer is 590 mgs.

### EXAMPLE 40

### Bi-Layered Tablet (Wet Granulation)

By using the process described in Example 39, a bi-layered tablet containing carbetapentane citrate in an immediate release layer and carbetapentane citrate, pseudoephedrine hydrochloride and chlorpheniramine maleate in a sustained release layer may be manufactured by using wet granulation:

| | |
|---|---|
| | Weight/tablet |
| Ingredients | (mgs) |

Layer 1 (Immediate Release)

| | |
|---|---|
| Carbetapentane Citrate | 30 |
| Silicified Microcrystalline cellulose | 126 |
| Povidone | 3 |
| Croscarmellose sodium | 10 |
| Magnesium Stearate | 1 |

Layer 2 (Sustained Release)

| | |
|---|---|
| Carbetapentane Citrate | 30 |
| Pseudoephedrine HCl | 60 |
| Chlorpheniramine Maleate | 8 |
| Microcrystalline Cellulose 102 | 30 |
| Lactose Monohydrate | 100 |
| Dicalcium Citrate | 100 |
| Povidone | 15 |
| Hydroxypropylmethylcellulose | 212 |
| Stearic Acid | 20 |
| Magnesium Stearate | 5 |
| Total | 750 |

The above examples 21-40 illustrate how to manufacture a bi-layered tablet containing an antitussive, i.e., carbetapentane citrate, in (at least) a first layer and an antihistamine and/or a decongestant and/or an expectorant in (at least) a second layer. Non-limiting examples of possible active ingredients (in addition to carbetapentane) in an exemplary range as described in the above Table 1 can be employed depending on the specific therapeutic effect desired.

### EXAMPLE 41

### Extended Release Suspension (Gel)

An extended release suspension (in the form of a gel) in accordance with the present invention which contains a carbetapentane citrate ion-exchange complex and promethazine hydrochloride is illustrated as follows (note that the carbetapentane citrate is used in a controlled release form since it has a shorter half-life than the promethazine hydrochloride):

| Ingredients | Amount/5 ml | |
|---|---|---|
| Carbetapentane Citrate | Equivalent to 60 mgs of | |
| Ion-Exchange Complex | Carbetapentane Citrate | |
| Promethazine HCl | 25 | mgs |
| Eudragit ® L 100 | 0.2 to 2.8 | grams |
| Glycerin | 315 | mgs |
| Polysorbate 80 | 1.5 | mgs |
| Carbomer (e.g., Carbopol ® 974) | 37.5 | mgs |
| Methyl Paraben | 9 | mgs |
| Propyl Paraben | 1 | mgs |
| Saccharin Sodium cryst., USP | 0.1 | mg |
| Artificial Grape Flavor | 5 | mgs |
| FD&C Red # 40 Dye | 0.5 | mgs |

| | |
|---|---|
| Sodium Hydroxide | q.s. |
| Water | q.s |

Procedure:

(1) Add the appropriate amount of sodium polystyrene sulphonate USP (e.g. Amberlite® IRP 69) to a carbetapentane citrate solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eudragit® L 100, Kollidon® MAE, Aquacoat® cPD) and dry the granules.

(5) Mill the granules, if needed.

(6) To an appropriate amount of water add the following ingredients and dissolve: promethazine hydrochloride, Carbomer (e.g., Carbopol® 974), glycerin, polysorbate 80, methyl paraben, propyl paraben, artificial grape flavor, FD&C red # 40 dye.

(7) Add milled granules.

(8) Add water to 95% of final volume.

(9) Agitate at suitable rate to avoid settling of the suspension and maintain a homogeneous product mixture.

(10) Neutralize the solution to form a gel using a 1N sodium hydroxide solution to a pH of 5 to 8. Add water to make final volume.

(11) Fill in suitable containers ensuring that the product is homogeneous throughout the filling operation.

### EXAMPLE 42

### Extended Release Suspension (Liquid)

An extended release suspension (in the form of a liquid) in accordance with the present invention which contains a carbetapentane tannate ion-exchange complex and promethazine hydrochloride is illustrated as follows:

| Ingredients | Amount/5 ml | |
|---|---|---|
| Carbetapentane Tannate | Equivalent to 30 mgs of | |
| Ion-Exchange Complex | Carbetapentane Tannate | |
| Promethazine HCl | 25 | mgs |
| Eudragit ® L 100 | 0.2 to 2.8 | grams |
| Silica, colloidal anhydrous, NF | 100 | mgs |
| Glycerin | 740 | mgs |
| Xylitol, NF | 800 | mgs |
| Sodium Citrate, USP | 100 | mgs |
| Saccharin Sodium cryst., USP, | 0.1 | mg |
| Sodium Benzoate | 7.5 | mgs |
| Citric Acid Monohydrate, USP | 8.0 | mgs |
| Artificial Grape Flavor | 5 | mgs |
| FD&C Red # 40 Dye | 0.5 | mgs |

| | |
|---|---|
| Water | q.s |

Manufacturing Process for 1000 L Batch:

Add the appropriate amount of sodium polystyrene sulphonate USP (e.g. Amberlite® IRP 69 or Amberlite® 88N) to a carbetapentane tannate solution. Stir the mix for 12 hrs to allow complete drug/resin complex formation. Separate and dry the insoluble drug/resin complex. Granulate the drug/resin complex with a delayed release/enteric polymer (e.g. Eudragit® L 100, Kollidon® MAE, Aquacoat® CPD) and dry the granules. Mill the granules, if needed.

In a suitably sized stainless steel vessel, dissolve saccharin sodium, sodium benzoate, citric acid, and sodium citrate in approximately 50 L of warm (about 45° C.), purified water. In another large stainless steel drum mix the silica, carbetapentane tannate ion-exchange complex, and promethazine hydrochloride until a uniform and consistent mixture is obtained. In a separate 1000 L stainless steel tank equipped with a suitably sized homogenizer/disperser add about 100 L of purified water. With the homogenizer on, add the silica mixture containing carbetapentane tannate ion-exchange complex, and promethazine hydrochloride. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes.

### EXAMPLE 43

### Extended Release Suspension (Liquid)

An extended release suspension (in the form of a liquid) in accordance with the present invention which contains a carbetapentane citrate ion-exchange complex, pseudoephedrine tannate and chlorpheniramine tannate is illustrated as follows:

| Ingredients | Amount/5 ml | |
|---|---|---|
| Carbetapentane Citrate | Equivalent to 20 mgs of | |
| Ion-Exchange Complex | Carbetapentane Citrate | |
| Pseudoephedrine Tannate | 75.0 | |
| Chlorpheniramine Tannate | 4.5 | |
| Eudragit ® L 100 | 0.2 to 2.8 | grams |
| Silica, colloidal anhydrous, NF | 100 | mgs |
| Glycerin | 740 | mgs |
| Xylitol, NF | 800 | mgs |
| Sodium Citrate, USP | 100 | mgs |
| Saccharin Sodium cryst., USP, | 0.1 | mg |
| Sodium Benzoate | 7.5 | mgs |
| Citric Acid Monohydrate, USP | 8.0 | mgs |
| Artificial Grape Flavor | 5 | mgs |
| FD&C Red # 40 Dye | 0.5 | mgs |

| | |
|---|---|
| Water | q.s |

Manufacturing Process for 1000 kg Batch:

Add the appropriate amount of sodium polystyrene sulphonate USP (e.g. Amberlite® IRP 69or Amberlite® 188N) to a carbetapentane citrate solution. Stir the mix for 12 hrs to allow complete drug/resin complex formation. Separate and dry the insoluble drug/resin complex. Granulate the drug/resin complex with a delayed release/enteric polymer (e.g. Eudragit® L 100, Kollidon® MAE, Aquacoat® CPD) and dry the granules. Mill the granules, if needed.

In a suitably sized stainless steel vessel, dissolve saccharin sodium, sodium benzoate, citric acid, and sodium citrate in approximately 50 L of warm (about 45° C.), purified water. In another large stainless steel drum mix the silica, carbetapentane citrate ion-exchange complex, pseudoephedrine tannate, and the chlorpheniramine tannate until a uniform and consistent mixture is obtained. In a separate 1000 L stainless steel tank equipped with a suitably sized homogenizer/disperser add about 100 L of purified water. With the homogenizer on, add the silica mixture containing carbetapentane citrate ion-exchange complex, pseudoephedrine tannate, and the chlorpheniramine tannate. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes.

### EXAMPLE 44

### Extended Release Gel

An extended release suspension which contains a carbetapentane citrate ion-exchange complex, a dexchlorpheniramine maleate ion-exchange complex and a phenylephrine hydrochloride ion-exchange complex is illustrated as follows:

| Ingredients | Amount/5 ml | |
|---|---|---|
| Carbetapentane Citrate | Equivalent to 20 mgs of | |
| Ion-Exchange Complex | Carbetapentane Citrate | |
| Dexchlorpheniramine Maleate | Equivalent to 4 mgs of | |
| Ion-Exchange Complex | Dexchlorpheniramine Maleate | |
| Phenylepherine HCl Ion-Exchange | Equivalent to 10 mgs of | |
| Complex | Phenylepherine HCl | |
| Eudragit ® L 100 | 0.2 to 2.8 | grams |
| Glycerin | 315 | mgs |
| Polysorbate 80 | 1.5 | mgs |
| Carbomer (e.g., Carbopol ® 974) | 15 | mgs |
| Methyl Paraben | 9 | mgs |
| Propyl Paraben | 1 | mgs |
| Artificial Grape Flavor | 5 | mgs |
| FD&C Red # 40 Dye | 0.5 | mgs |

| | |
|---|---|
| Water | q.s |

The formula described above serves as a non-limiting example. Any active drug which is in the form of a salt can be incorporated as an ion-exchange resin complex.

Procedure:

(1) Add the appropriate amount of sodium polystyrene sulphonate USP (e.g. Amberlite® IRP 69 or Amberlite® 188N) to a carbetapentane citrate, dexchlorpheniramine maleate and phenylephrine HCl solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eugragit® L 100, Kollidon® MAE, Aquacoa®t cPD) and dry the granules.

(5) Mill the granules, if needed.

(6) To an appropriate amount of water add the following ingredients and dissolve: Carbomer (e.g., Carbopol® 974), glycerin, polysorbate 80, methyl paraben, propyl paraben, artificial grape flavor, FD&C red # 40 dye.

(7) Add milled granules.

(8) Add water to 95% of the final volume.

(9) Agitate at suitable rate to avoid settling of the suspension and maintain a homogeneous product mixture.

(10) Neutralize the solution to form a gel using a 1N sodium hydroxide solution to a pH of 5 to 8. Add enough water to make up to the final volume.

(11) Fill in suitable containers ensuring that the product is homogeneous throughout the filling operation.

### EXAMPLE 45

### Suspension Formula

A suspension formula which comprises carbetapentane citrate and phenylephrine tannate is illustrated as follows:

| | g/100 mL = | | kg/batch = | |
|---|---|---|---|---|
| Ingredients | 120 g | | 1000 kg | |
| Carbetapentane Citrate | 0.500 | | | |
| Phenylepherine Tannate | 0.800 | | 6.667 | |
| Silica, colloidal anhydrous, NF | 1.73 | | 14.417 | |
| Hydroxyethylcellulose, NF | 0.05 | | 0.417 | |
| Sorbitol Solution 70% | 34.00 | | 283.333 | |
| (non-crystallizing), NF | | | | |
| Glycerol | 14.75 | | 122.917 | |
| Xylitol, NF | 16.00 | | 133.333 | |
| Sodium Citrate, USP | 2.00 | | 16.667 | |
| Saccharin Sodium cryst., USP, | 0.01 | | 0.083 | |
| Sodium Benzoate, NF | 0.15 | | 1.250 | |
| Citric Acid Monohydrate, USP | 0.16 | | 1.333 | |
| Strawberry Flavor | 0.15 | | 1.250 | |
| Banana Flavor | 0.15 | | 1.250 | |
| Purified Water | 49.55 | | 412.917 | |
| Total Amount | 120.000 | g | 1000.000 | kg |

Manufacturing Process for 1000 kg Batch: bbb

In a suitably sized stainless steel vessel, dissolve saccharin sodium, sodium benzoate, citric acid, and sodium citrate in approximately 50 L of warm (about 45° C.), purified water. In another large stainless steel drum mix the silica, carbetapentane citrate, and micronized phenylephrine tannate until a uniform and consistent mixture is obtained. In a separate 1000 L stainless steel tank equipped with a suitably sized homogenizer/disperser add about 100 L of purified water. With the homogenizer on, add the silica mixture containing phenylephrine tannate and carbetapentane citrate. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes. Filter product through a 10 micron filter and fill in appropriately sized containers.

To make products with other agents such as antihistamines, decongestants, or expectorants, one or more combinations of each of the ingredients in a range as described in Table 1 above can be made depending on the specific therapeutic effect desired.

### EXAMPLE 46

### Liquid Formula

A liquid dosage form which comprises carbetapentane citrate and phenylephrine hydrochloride is illustrated as follows:

| Ingredients | Per 5 mL | | Per 425 L | |
|---|---|---|---|---|
| Carbetapentane Citrate USP | 30 | mg | 2.55 | kg |
| Phenylepherine Hydrochloride USP | 10.0 | mg | 0.850 | kg |
| Methyl Paraben USP | 9.0 | mg | 0.765 | kg |
| Propyl Paraben USP | 1.0 | mg | 0.085 | kg |
| Propylene Glycol USP | 259 | mg | 22.016 | kg |
| Saccharin Sodium USP | 3.18 | mg | 0.270 | kg |
| Citric Acid USP | 5.0 | mg | 0.425 | kg |
| Strawberry Flavor | 10 | mg | 0.850 | kg |
| Banana Flavor | 10 | mg | 0.850 | kg |
| Sorbitol Solution 70% USP | 3212.5 | mg | 273.1 | kg |
| Purified Water, as required to q.s. to | 5.0 | mL | 425 | L |

Manufacturing Process for 425 L Batch Size:

In a suitably sized stainless steel vessel, dissolve methyl paraben and propyl paraben in approximately 50 L of warm (about 45° C.), purified water. Add about half of the propylene glycol and mix for about 1 hr. In a separate 1000 L stainless steel tank equipped with a suitably sized agitator, add about 50 L of purified water. With the agitator on, add phenylephrine hydrochloride, carbetapentane citrate, saccharin sodium and citric acid and dissolve. Add the previously prepared paraben/propylene glycol solution to the 1000 L tank. Rinse the first vessel with about 2 L of water and transfer the rinsate to the 1000 L tank. Add the remaining propylene glycol to a suitably sized stainless steel vessel and dissolve the strawberry and banana flavors. Transfer this to the 1000 L tank. Rinse the container with 2 L of purified water and transfer to the 1000 L tank. With the agitator on, add the sorbitol solution 70% to the 1000 L tank. In a suitably sized stainless steel vessel, dissolve the carbetapentane citrate in about 5 L of purified water and transfer to the 1000 L tank. Rinse the container with about 2 L of purified water and transfer to the 1000 L tank. Stop the agitator and let the solution stand for 15 minutes. QS to 425 L with purified water. Filter product through a 1 micron filter and fill in appropriately sized containers.

To make products with other antihistamines, decongestants, or expectorants, one or more combinations of each of the ingredients in a range as described in Table 1 above can be made depending on the specific therapeutic effect desired.

### EXAMPLE 47

### Bi-Layered Tablet (Wet Granulation)

A bi-layered tablet in accordance with the present invention which comprises phenylephrine hydrochloride in a first sustained release layer and guaifenesin in a second sustained release layer is illustrated as follows:

| | | |
|---|---|---|
| | Weight/tablet | Weight |
| Ingredients | (mg) | (kg) |

Layer 1 (Sustained release)

| | | |
|---|---|---|
| Phenylepherine HCl | 60.0 | 6.0 |
| Methocel K15M | 100.0 | 10.0 |
| Silicified Microcrystalline Cellulose | 50.0 | 5.0 |
| Eudragit NE | 42.0 | 4.2 |
| Magnesium Stearate | 8.0 | 8.0 |

Layer 2 (Sustained release)

| | | |
|---|---|---|
| Guaifenesin | 600.0 | 60.0 |
| Microcrystalline Cellulose (PH 102) | 45.0 | 45.0 |
| Eudragit NE | 15.0 | 15.0 |
| Methocel K4M Premium | 140.0 | 14.0 |
| Stearic Acid | 20.0 | 2.0 |
| Magnesium Stearate | 5.0 | 0.5 |
| Total | 1085.0 | 108.5 |

Procedure:

(a) Sustained release layer #1: Mix phenylephrine hydrochloride in Methocel®K15M and silicified microcrystalline cellulose in a high shear mixer/granulator for 10 minutes. Granulate the above blend using a Eudragit® NE (30%). Dry the granulation until the LOD (weight loss on drying) is less than 2.0%. Screen granules through a US sieve size # 14. Add the granules and the prescreened magnesium stearate in a V shaped blender and mix for 3 minutes.

(b) Sustained release layer #2: Screen all ingredients through a US sieve size # 30. Mix guaifenesin, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 260 mg and layer #2 is 825 mg. Capsules may be manufactured by filling the same proportions into capsules.

### EXAMPLE 48

### Bi-Layered Tablet (Wet Granulation)

A bi-layered tablet in accordance with the present invention which comprises promethazine hydrochloride in an immediate release layer and phenylephrine hydrochloride in a sustained release layer is illustrated as follows:

| | | |
|---|---|---|
| | Weight/tablet | Weight batch |
| Ingredients | (mg) | (Kg) |

Layer 1 (Immediate release)

| | | |
|---|---|---|
| Promethazine HCl | 25.0 | 25.0 |
| Silicified Microcrystalline Cellulose | 161.0 | 161.0 |
| Povidone | 3.0 | 3.0 |
| Croscarmellose Sodium | 10.0 | 10.0 |
| Magnesium Stearate | 1.0 | 1.0 |

Layer 2 (Sustained release)

| | | |
|---|---|---|
| Phenylepherine hydrochloride | 30.0 | 30.0 |
| Microcrystalline Cellulose (PH 102) | 25.0 | 25.0 |
| Dicalcium Phosphate | 50.0 | 50.0 |
| Povidone | 15.0 | 15.0 |
| Methocel K4M Premium | 205.0 | 205.0 |
| Stearic Acid | 20.0 | 20.0 |
| Magnesium Stearate | 5.0 | 5.0 |
| Total | 550.0 | 550.0 |

Procedure:

(a) Immediate release layer #1: Mix the promethazine HCl, silicified microcrystalline cellulose and croscarmellose sodium, in a high shear mixer/granulator for 10 minutes. Granulate the above blend using a 30% povidone solution (3.0 g povidone in 10.0 g purified water). Dry the granulation until the LOD is less than 2.0%. Screen granules through a US sieve size # 14. Add the granules and the prescreened magnesium stearate in a V shaped blender and mix for 3 minutes.

(b) Sustained release layer #2: Mix the phenylephrine hydrochloride, microcrystalline cellulose PH 102, dicalcium phosphate, Methocel K4M Premium and stearic acid in a high shear mixer/granulator for 10 minutes. Granulate the above blend using a 30% povidone solution (15.0 g povidone in 50.0 g purified water). Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 200 mg and layer #2 is 350 mg. Capsules may be manufactured by filling the same proportions into capsules.

### EXAMPLE 49

### Bi-Layered Tablet (Wet Granulation)

A bi-layered tablet in accordance with the present invention which comprises phenylephrine hydrochloride and carbinoxamine maleate in a first sustained release layer and phenylephrine hydrochloride in a second sustained release layer is illustrated as follows:

| | | |
|---|---|---|
| | Weight/tablet | Weight batch |
| Ingredients | (mg) | (Kg) |

Layer 1 (Sustained release)

| | | |
|---|---|---|
| Phenylepherine HCl | 75.0 | 75.0 |
| Carbinoxamine Maleate | 8.0 | 8.0 |
| Methocel K4M | 120.0 | 120.0 |
| Silicified Microcrystalline Cellulose | 30.0 | 30.0 |
| Eudragit NE | 15.0 | 15.0 |
| Magnesium Stearate | 3.0 | 3.0 |

Layer 2 (Sustained release)

| | | |
|---|---|---|
| Phenylepherine hydrochloride | 30.0 | 30.0 |
| Microcrystalline Cellulose (PH 102) | 65.0 | 65.0 |
| Eudragit NE | 20.0 | 20.0 |
| Methocel K4M Premium | 113.0 | 113.0 |
| Stearic Acid | 20.0 | 20.0 |
| Magnesium Stearate | 5.0 | 5.0 |
| Total | 500.0 | 500.0 |

Procedure:

(a) Sustained release layer #1: Mix the phenylephrine HCl, carbinoxamine maleate, Methocel®K4M and silicified microcrystalline cellulose in a high shear mixer/granulator for 10 minutes. Granulate the above blend using a Eudragit® NE (30%). Dry the granulation until the LOD is less than 2.0%. Screen granules through a US sieve size # 14. Add the granules and the prescreened magnesium stearate in a V shaped blender and mix for 3 minutes.

(b) Sustained release layer #2: Screen all ingredients through a US sieve size # 30. Mix the phenylephrine hydrochloride, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 250 mg and layer #2 is 250 mg. Capsules may be manufactured by filling the same proportions into capsules.

### EXAMPLE 50

### Bi-Layered Tablet (Wet Granulation)

A bi-layered tablet in accordance with the present invention which comprises phenylephrine hydrochloride and diphenhydramine hydrochloride in a first sustained release layer and codeine phosphate in a second sustained release layer is illustrated as follows:

| | | |
|---|---|---|
| | Weight/tablet | Weight batch |
| Ingredients | (mg) | (Kg) |

Layer 1 (Sustained release)

| | | |
|---|---|---|
| Phenylepherine HCl | 120.0 | 120.0 |
| Diphenhydramine HCl | 12.0 | 12.0 |
| Methocel K4M | 200.0 | 200.0 |
| Silicified Microcrystalline Cellulose | 35.0 | 35.0 |
| Eudragit NE | 20.0 | 20.0 |
| Magnesium Stearate | 3.0 | 3.0 |

Layer 2 (Sustained release)

| | | |
|---|---|---|
| Codeine Phosphate | 30.0 | 30.0 |
| Microcrystalline Cellulose (PH 102) | 55.0 | 55.0 |
| Eudragit NE | 15.0 | 31.7 |
| Methocel K4M Premium | 138.0 | 138.0 |
| Stearic Acid | 20.0 | 20.0 |
| Magnesium Stearate | 2.0 | 2.0 |
| Total | 650.0 | 650.0 |

Procedure:

(a) Sustained release layer #1: Mix the phenylephrine HCl, diphenhydramine hydrochloride, Methocel®K4M and silicified microcrystalline cellulose in a high shear mixer/granulator for 10 minutes. Granulate the above blend using a Eudragit® NE (30%) Dry the granulation until the LOD is less than 2.0%. Screen granules through a US sieve size # 14. Add the granules and the prescreened magnesium stearate in a V shaped blender and mix for 3 minutes.

(b) Sustained release layer #2: Screen all ingredients through a US sieve size # 30. Mix the codeine phosphate, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 390 mg and layer #2 is 260 mg. Capsules may be manufactured by filling the same proportions into capsules.

### EXAMPLE 51

### Bi-Layered Tablet (Wet Granulation)

A bi-layered tablet in accordance with the present invention which comprises carbinoxamine maleate in a first sustained release layer and phenylephrine hydrochloride in a second sustained release layer is illustrated as follows:

| | | |
|---|---|---|
| | Weight/tablet | Weight batch |
| Ingredients | (mg) | (Kg) |

Layer 1 (Sustained release)

| | | |
|---|---|---|
| Carbinoxamine Maleate | 8.0 | 8.0 |
| Lactose Monohydrate | 61.0 | 61.0 |
| Methocel K4M | 70.0 | 70.0 |
| Silicified Microcrystalline Cellulose | 39.0 | 39.0 |
| Eudragit NE | 20.0 | 20.0 |
| Magnesium Stearate | 2.0 | 2.0 |

Layer 2 (Sustained release)

| | | |
|---|---|---|
| Phenylepherine hydrochloride. | 30.0 | 30.0 |
| Microcrystalline Cellulose (PH 102) | 45.0 | 45.0 |
| Eudragit NE | 15.0 | 15.0 |
| Methocel K4M Premium | 103.0 | 103.0 |
| Stearic Acid | 20.0 | 20.0 |
| Magnesium Stearate | 2.0 | 2.0 |
| Total | 415.0 | 415.0 |

Procedure:

(a) Sustained release layer # 1: Mix the carbinoxamine maleate, Methocel®K4M, lactose monohydrate and silicified microcrystalline cellulose in a high shear mixer/granulator for 10 minutes. Granulate the above blend using a Eudragit® NE (30%). Dry the granulation until the LOD is less than 2.0%. Screen granules through a US sieve size # 14. Add the granules and the prescreened magnesium stearate in a V shaped blender and mix for 3 minutes.

(b) Sustained release layer #2: Screen all ingredients through a US sieve size # 30. Mix the phenylephrine hydrochloride, microcrystalline cellulose PH 102, and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 200 mg and layer #2 is 215 mg. Capsules may be manufactured by filling the same proportions into capsules.

### EXAMPLE 52

### Bi-Layered Tablet (Direct Compression)

A bi-layered tablet in accordance with the present invention which comprises promethazine hydrochloride (longer half-life drug) in an immediate release layer and phenylephrine hydrochloride (shorter half-life drug) in a sustained release layer is illustrated as follows:

| | | |
|---|---|---|
| | Weight/tablet | Weight batch |
| Ingredients | (mg) | (Kg) |

Layer 1 (Immediate release)

| | | |
|---|---|---|
| Promethazine HCl | 25.0 | 25.0 |
| Silicified Microcrystalline | 114.0 | 114.0 |
| Cellulose | | |
| Sodium Starch Glycolate | 10.0 | 10.0 |
| Magnesium Stearate | 1.0 | 1.0 |

Layer 2 (Sustained release)

| | | |
|---|---|---|
| Phenylepherine hydrochloride | 60.0 | 60.0 |
| Lactose Monohydrate | 50.0 | 50.0 |
| Dicalcium Phosphate | 50.0 | 50.0 |
| Kollidon SR | 220.0 | 220.4 |
| Stearic acid | 15.0 | 15.0 |
| Magnesium Stearate | 5.0 | 5.0 |
| Total | 550.0 | 550.0 |

Procedure:

(a) Immediate release layer #1: Screen all ingredients through a US sieve size # 30. Blend the promethazine hydrochloride, microcrystalline cellulose and sodium starch glycolate for 20 minutes. Add magnesium stearate to the above blend and mix for an additional time of three minutes.

(b) Sustained release layer #2: Blend the phenylephrine hydrochloride, lactose monohydrate, dicalcium phosphate and Kollidon® SR for 20 minutes. Add stearic acid and magnesium stearate to the above blend and mix for an additional time of three minutes.

Manufacture bi-layered tablets using a rotary bi-layer tablet press where in each tablet the immediate release layer #1 is 150 mg and the sustained release layer #2 is 400′ mg. Capsules may be manufactured by filling the same proportions into capsules.

### EXAMPLE 53

### Bi-Layered Tablet (Wet Granulation)

A bi-layered tablet in accordance with the present invention which comprises phenylephrine tannate and chlorpheniramine tannate in an immediate release layer and phenylephrine tannate in a sustained release layer is illustrated as follows:

| | | |
|---|---|---|
| | Weight/tablet | Weight batch |
| Ingredients | (mg) | (Kg) |

Layer 1 (Immediate release)

| | | |
|---|---|---|
| Phenylepherine Tannate | 60.0 | 60.0 |
| Chlorpheniramine Tannate | 8.0 | 8.0 |
| Silicified Microcrystalline Cellulose | 208.0 | 208.0 |
| Povidone | 3.0 | 3.0 |
| Croscarmellose Sodium | 10.0 | 10.0 |
| Magnesium Stearate | 1.0 | 1.0 |

Layer 2 (Sustained release)

| | | |
|---|---|---|
| Phenylepherine tannate | 30.0 | 30.0 |
| Microcrystalline Cellulose (PH 102) | 30.0 | 30.0 |
| Dicalcium Phosphate | 100.0 | 100.0 |
| Povidone | 15.0 | 15.0 |
| Methocel K4M Premium | 210.0 | 210.0 |
| Stearic Acid | 20.0 | 20.0 |
| Magnesium Stearate | 5.0 | 5.0 |
| Total | 700.0 | 700.0 |

Procedure:

(a) Immediate release layer #1: Mix the phenylephrine tannate, chlorpheniramine tannate, silicified microcrystalline cellulose and croscarmellose sodium, in a high shear mixer/granulator for 10 minutes. Granulate the above blend using a 30% povidone solution (3.0 Kg povidone in 7.0 Kg purified water). Dry the granulation until the LOD is less than 2.0%. Screen granules through a US sieve size # 14. Add the granules and the prescreened magnesium stearate in a V shaped blender and mix for 3 minutes.

(b) Sustained release layer #2: Mix the Phenylepherine tannate, microcrystalline cellulose PH 102, dicalcium phosphate, Methocel K4M Premium and stearic acid in a high shear mixer/granulator for 10 minutes. Granulate the above blend using a 30% povidone solution (15.0 Kg povidone in 35.0 Kg purified water). Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 290 mg and layer #2 is 410 mg. Capsules may be manufactured by filling the same proportions into capsules.

### EXAMPLE 54

### Bi-Layered Tablet (Wet Granulation)

A bi-layered tablet in accordance with the present invention which comprises diphenhydramine hydrochloride in an immediate release layer and phenylephrine hydrochloride in a sustained release layer is illustrated as follows:

| | | |
|---|---|---|
| | Weight/tablet | Weight batch |
| Ingredients | (mg) | (Kg) |

Layer 1 (Immediate release)

| | | |
|---|---|---|
| Diphenhydramine HCl | 100.0 | 100.0 |
| Silicified Microcrystalline Cellulose | 86.0 | 86.0 |
| Povidone | 3.0 | 3.0 |
| Croscarmellose Sodium | 10.0 | 10.0 |
| Magnesium Stearate | 1.0 | 1.0 |

Layer 2 (Sustained release)

| | | |
|---|---|---|
| Phenylepherine HCl | 75.0 | 75.0 |
| Microcrystalline Cellulose (PH 102) | 30.0 | 30.0 |
| Dicalcium Phosphate | 30.0 | 30.0 |
| Povidone | 15.0 | 15.0 |
| Methocel K4M Premium | 178.0 | 178.0 |
| Stearic Acid | 20.0 | 20.0 |
| Magnesium Stearate | 2.0 | 2.0 |
| Total | 550.0 | 550.0 |

Procedure:

(a) Immediate release layer #1: Mix the diphenydramine hydrochloride, silicified microcrystalline cellulose and croscarmellose sodium, in a high shear mixer/granulator for 10 minutes. Granulate the above blend using a 30% povidone solution (3.0 g povidone in 10.0 g purified water). Dry the granulation until the LOD is less than 2.0%. Screen granules through a US sieve size # 14. Add the granules and the prescreened magnesium stearate in a V shaped blender and mix for 3 minutes.

(b) Sustained release layer #2: Mix the phenylephrine hydrochloride, microcrystalline cellulose PH 102, dicalcium phosphate, Methocel K4M Premium and stearic acid in a high shear mixer/granulator for 10 minutes. Granulate the above blend using a 30% povidone solution (15.0 g povidone in 50.0 g purified water). Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 200 mg and layer #2 is 350 mg. Capsules may be manufactured by filling the same proportions into capsules.

### EXAMPLE 55

### Bi-Layered Tablet (Direct Compression)

A bi-layered tablet in accordance with the present invention which comprises guaifenesin in a first sustained release layer and phenylephrine hydrochloride in a second sustained release layer is illustrated as follows:

| | | |
|---|---|---|
| | Weight/tablet | Weight batch |
| Ingredients | (mg) | (Kg) |

Layer 1 (Sustained release)

| | | |
|---|---|---|
| Guaifenesin | 600.0 | 300.0 |
| Methocel K15M | 200.0 | 100.0 |
| Silicified Microcrystalline | 72.0 | 36.0 |
| Cellulose | | |
| Magnesium Stearate | 8.0 | 4.0 |

Layer 2 (Sustained release)

| | | |
|---|---|---|
| Phenylepherine hydrochloride | 60.0 | 30.0 |
| Dicalcium Phosphate | 70.0 | 35.0 |
| Kollidon SR | 172.0 | 86.0 |
| Stearic acid | 15.0 | 7.5 |
| Magnesium Stearate | 3.0 | 1.5 |
| Total | 1200.0 | 600.0 |

Procedure:

(a) Sustained release layer #1: Screen all ingredients through a US sieve size # 30. Blend the guaifenesin, Methocel® K15M and silicified microcrystalline cellulose for 25 minutes. Add magnesium stearate to the above blend and mix for an additional time of three minutes.

(b) Sustained release layer #2: Blend the phenylephrine hydrochloride, dicalcium phosphate and Kollidon® SR for 20 minutes. Add stearic acid and magnesium stearate to the above blend and mix for an additional time of three minutes.

Manufacture bi-layered tablets using a rotary bi-layer tablet press where in each tablet layer #1 is 880 mg and layer #2 is 320 mg. Capsules may be manufactured by filling the same proportions into capsules.

### EXAMPLE 56

### Bi-Layered Tablet (Wet Granulation)

A bi-layered tablet in accordance with the present invention which comprises brompheniramine maleate in a first sustained release layer and phenylephrine hydrochloride in a second sustained release layer is illustrated as follows:

| | | |
|---|---|---|
| | Weight/tablet | Weight batch |
| Ingredients | (mg) | (Kg) |

Layer 1 (Sustained release)

| | | |
|---|---|---|
| Brompheniramine Maleate | 6.0 | 6.0 |
| Methocel K15M | 94.0 | 94.0 |
| Silicified Microcrystalline Cellulose | 50.0 | 50.0 |
| Eudragit NE | 42.0 | 42.0 |
| Magnesium Stearate | 8.0 | 8.0 |

Layer 2 (Sustained release)

| | | |
|---|---|---|
| Phenylepherine HCl | 60.0 | 60.0 |
| Microcrystalline Cellulose (PH 102) | 45.0 | 45.0 |
| Eudragit NE | 15.0 | 15.0 |
| Methocel K4M Premium | 108.0 | 108.0 |
| Stearic Acid | 20.0 | 20.0 |
| Magnesium Stearate | 2.0 | 2.0 |
| Total | 450.0 | 450.0 |

Procedure:

(a) Sustained release layer #1: Mix the brompheniramine maleate, Methocel®K15M and silicified microcrystalline cellulose in a high shear mixer/granulator for 10 minutes. Granulate the above blend using a Eudragit® NE (30%). Dry the granulation until the LOD is less than 2.0%. Screen granules through a US sieve size # 14. Add the granules and the prescreened magnesium stearate in a V shaped blender and mix for 3 minutes.

(b) Sustained release layer #2: Screen all ingredients through a US sieve size # 30. Mix the phenylephrine hydrochloride, microcrystalline cellulose PH 102, dicalcium phosphate and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 200 mg and layer #2 is 250 mg. Capsules may be manufactured by filling the same proportions into capsules.

### EXAMPLE 57

### Bi-Layered Tablet (Wet Granulation)

A bi-layered tablet in accordance with the present invention which comprises guaifenesin in a first sustained release layer and phenylephrine hydrochloride in a second sustained release layer is illustrated as follows:

| | | |
|---|---|---|
| | Weight/tablet | Weight batch |
| Ingredients | (mg) | (Kg) |

Layer 1 (Sustained release)

| | | |
|---|---|---|
| Guaifenesin | 1000.0 | 400.0 |
| Methocel K15M | 200.0 | 80.0 |
| Silicified Microcrystalline Cellulose | 40.0 | 16.0 |
| Eudragit NE | 50.0 | 20.0 |
| Magnesium Stearate | 10.0 | 4.0 |

Layer 2 (Sustained release)

| | | |
|---|---|---|
| Phenylepherine hydrochloride | 30.0 | 12.0 |
| Microcrystalline Cellulose (PH 102) | 45.0 | 18.0 |
| Eudragit NE | 15.0 | 6.0 |
| Methocel K4M Premium | 100.0 | 40.0 |
| Stearic Acid | 20.0 | 8.0 |
| Magnesium Stearate | 2.0 | 0.5 |
| Total | 1510.0 | 604.0 |

Procedure:

(a) Sustained release layer #1: Mix the guaifenesin, Methocel®K15M and silicified microcrystalline cellulose in a high shear mixer/granulator for 10 minutes. Granulate the above blend using a Eudragit® NE (30%). Dry the granulation until the LOD is less than 2.0%. Screen granules through a US sieve size # 14. Add the granules and the prescreened magnesium stearate in a V shaped blender and mix for 3 minutes.

(b) Sustained release layer #2: Screen all ingredients through a US sieve size # 30. Mix the phenylephrine HCl, microcrystalline cellulose PH 102 and stearic acid in a high shear mixer/granulator for 10 minutes. Granulate the above blend using a Eudragit® NE (30%). Add the Methocel®K4M to the granulator and post mix for 5 minutes. Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 1300 mg and layer #2 is 210 mg. Capsules may be manufactured by filling the same proportions into capsules.

### EXAMPLE 58

### Bi-Layered Tablet (Direct Compression)

A bi-layered tablet in accordance with the present invention which comprises phenylephrine hydrochloride in a first immediate release layer and phenylephrine hydrochloride and diphenhydramine hydrochloride in a second sustained release layer is illustrated as follows:

| | | |
|---|---|---|
| | Weight/tablet | Weight batch |
| Ingredients | (mg) | (Kg) |

Layer 1 (Immediate release)

| | | |
|---|---|---|
| Phenylepherine hydrochloride | 30.0 | 30.0 |
| Silicified Microcrystalline | 160.0 | 160.0 |
| Cellulose | | |
| Sodium Starch Glycolate | 9.0 | 9.0 |
| Magnesium Stearate | 1.0 | 1.0 |

Layer 2 (Sustained release)

| | | |
|---|---|---|
| Phenylepherine HCl | 60.0 | 60.0 |
| Diphenhydramine HCl | 60.0 | 60.0 |
| Dicalcium Phosphate | 100.0 | 100.0 |
| Kollidon SR | 220.0 | 220.0 |
| Stearic acid | 16.0 | 16.0 |
| Magnesium Stearate | 4.0 | 4.0 |
| Total | 660.0 | 660.0 |

Procedure:

(a) Immediate release Layer #1: Screen all ingredients through a US sieve size # 30. Preblend a portion (about a third of the total) of the silicified microcrystalline cellulose and all the phenylephrine hydrochloride for the immediate release layer for 15 minutes. Add sodium starch glycolate and mix for additional 10 minutes. Add magnesium stearate to the above blend and mix for three minutes.

(b) Sustained release layer #2: Screen all ingredients through a US sieve size # 30. Preblend a portion of the Kollidon SR (about a third of the total) and all the diphenhydramine hydrochloride for 15 minutes. Add the remaining Kollidon SR, phenylephrine hydrochloride and dicalcium phosphate to the above preblend and mix for additional 20 minutes. Add stearic acid and magnesium stearate to the above blend and mix for three minutes.

Manufacture bi-layered tablets using a rotary bi-layer tablet press where in each tablet the immediate release layer is 200 mg and the sustained release layer is 460 mg.

### EXAMPLE 59

### Bi-Layered Tablet (Direct Compression)

By using the process described in Example 58, a bi-layered tablet which contains phenylephrine hydrochloride in an immediate release layer and phenylephrine hydrochloride and dexbrompheniramine maleate in a sustained release layer (see the formula below) may be manufactured by using direct compression:

| | |
|---|---|
| Ingredients | Weight/tablet (mg) |

Layer 1 (Immediate Release)

| | |
|---|---|
| Phenylepherine hydrochloride | 10.0 |
| Silicified Microcrystalline Cellulose | 133.5 |
| Sodium Starch Glycolate | 15.0 |
| Magnesium Stearate | 1.5 |

Layer 2 (Sustained Release)

| | |
|---|---|
| Phenylepherine hydrochloride | 40.0 |
| Dexbrompheniramine maleate | 6.0 |
| Dicalcium Phosphate | 100.0 |
| Kollidon SR | 244.0 |
| Stearic Acid | 7.0 |
| Magnesium Stearate | 3.0 |
| Total | 560.0 |

### EXAMPLE 60

### Bi-Layered Tablet (Wet Granulation)

A bi-layered tablet in accordance with the present invention which comprises phenylephrine hydrochloride in an immediate release layer and phenylephrine hydrochloride and chlorpheniramine maleate in a sustained release layer is illustrated as follows:

| | | |
|---|---|---|
| | Weight/tablet | Weight batch |
| Ingredients | (mg) | (Kg) |

Layer 1 (Immediate release)

| | | |
|---|---|---|
| Phenylepherine hydrochloride | 10.0 | 10.0 |
| Silicified Microcrystalline Cellulose | 111.0 | 111.0 |
| Povidone | 3.0 | 3.0 |
| Croscarmellose Sodium | 10.0 | 10.0 |
| Magnesium Stearate | 1.0 | 1.0 |

Layer 2 (Sustained release)

| | | |
|---|---|---|
| Phenylepherine HCl | 60.0 | 60.0 |
| Chlorpheniramine Maleate | 8.0 | 8.0 |
| Microcrystalline Cellulose (PH 102) | 30.0 | 30.0 |
| Dicalcium Phosphate | 100.0 | 100.0 |
| Povidone | 15.0 | 15.0 |
| Methocel K4M Premium | 212.0 | 212.0 |
| Stearic Acid | 20.0 | 20.0 |
| Magnesium Stearate | 5.0 | 5.0 |
| Total | 585.0 | 585.0 |

Procedure:

(a) Immediate release layer #1: Screen all ingredients through a US sieve size # 30. Blend the phenylephrine hydrochloride, silicified microcrystalline cellulose, and croscarmellose sodium in a high shear mixer/granulator for 10 minutes. Granulate the above blend using a 30% povidone solution (4.3 Kg povidone in 10.0 Kg purified water). Dry the granulation until the LOD is less than 2.0%. Screen granules through a US sieve size # 14. Add granules and the prescreened magnesium stearate to the above blend and mix for 3 minutes.

(b) Sustained release layer #2: Screen all ingredients through a US sieve size # 30. Blend the phenylephrine hydrochloride, chlorpheniramine maleate, microcrystalline cellulose PH 102, dicalcium phosphate and Methocel K4M Premium and stearic acid in a high shear mixer/granulator for 10 minutes. Granulate the above blend using a 30% povidone solution (15.0 Kg povidone in 35.0 Kg purified water). Dry the granulation until the LOD is less than 2.0%. Screen granules through a US sieve size # 14. Add granules and the prescreened magnesium stearate to the above blend and mix for 3 minutes.

Manufacture bi-layered tablets using a rotary bi-layer tablet press where in each tablet the immediate release layer is 135 mg and the sustained release layer is 450 mg.

### EXAMPLE 61

### Bi-Layered Tablet (Wet Granulation)

By using the process described in Example 60, a bi-layered tablet containing phenylephrine hydrochloride in an immediate release layer and phenylephrine hydrochloride and chlorpheniramine maleate in a sustained release layer may be manufactured by using wet granulation:

| | |
|---|---|
| Ingredients | Weight/tablet (mg) |

Layer 1 (Immediate Release)

| | |
|---|---|
| Phenylepherine hydrochloride | 30.0 |
| Silicified Microcrystalline cellulose | 129.5 |
| Povidone | 4.0 |
| Croscarmellose sodium | 15.0 |
| Magnesium Stearate | 1.5 |

Layer 2 (Sustained Release)

| | |
|---|---|
| Phenylepherine HCl | 60.0 |
| Chlorpheniramine Maleate | 8.0 |
| Microcrystalline Cellulose 102 | 30.0 |
| Lactose Monohydrate | 100.0 |
| Dicalcium Phosphate | 100.0 |
| Povidone | 15.0 |
| Hydroxypropylmethylcellulose | 212.0 |
| Stearic Acid | 20.0 |
| Magnesium Stearate | 5.0 |
| Total | 730.0 |

### EXAMPLE 62

### Bi-Layered Tablet (Wet Granulation)

A bi-layered tablet in accordance with the present invention which comprises phenylephrine hydrochloride in a sustained release layer and carbetapentane citrate in a immediate release layer is illustrated as follows:

Formula

| | | Amounts | |
|---|---|---|---|
| Process Steps | Ingredients | Dose (mg) | Scale-Up |

Sustained Release Layer

| | | | |
|---|---|---|---|
| Wet Mix | RMI - PURIFIED WATER | 48.000 | 15.000 kg |
| Wet Mix | RMI - POVIDONE K-30 USP | 12.000 | 3.750 kg |
| Pre-blend | RMI - PHENYLEPHRINE HCl USP | 40.000 | 12.500 kg |
| | UNMIC | | |
| Pre-blend | RMI - CAL PHOSPHATE DIBASIC | 25.000 | 7.813 kg |
| | DIHYD | | |
| Pre-blend | RMI - PROSOLV SMCC 90 | 125.000 | 39.063 kg |
| Pre-blend | RMI - METHOCEL K4M PREMIUM USP | 50.000 | 15.625 kg |
| Final blend | RMI - METHOCEL K4M PREMIUM USP | 144.000 | 45.000 kg |
| Lube blend | RMI - MAGNESIUM STEARATE NF | 4.000 | 1.250 kg |
| Layer Weight: | | 400.000 | |

Immediate Release Layer

| | | | |
|---|---|---|---|
| Wet Mix | RMI - PURIFIED WATER | 24.000 | 7.500 kg |
| Wet Mix | RMI - POVIDONE K-30 USP | 6.000 | 1.875 kg |
| Pre-blend | RMA - CARBETAPENTANE CITRATE | 40.000 | 12.500 kg |
| Pre-blend | RMI - PROSOLV SMCC 90 | 100.000 | 31.250 kg |
| Pre-blend | RMI - LACTOSE MONOHYDRATE #316 | 40.000 | 12.500 kg |
| Pre-blend | RMI - SODIUM STARCH GLYCOLATE | 8.000 | 2.500 kg |
| Pre-blend | DYE - FD&C BLUE #1 ALUMINUM | 0.156 | 0.048 kg |
| | LAKE | | |
| Final blend | RMI - PROSOLV SMCC 90 | 145.688 | 45.528 kg |
| Final blend | RMI - LACTOSE MONOHYDRATE #316 | 40.000 | 12.500 kg |
| Final blend | RMI - SODIUM STARCH GLYCOLATE | 16.000 | 5.000 kg |
| Final blend | DYE - FD&C BLUE #1 ALUMINUM | 0.156 | 0.048 kg |
| | LAKE | | |
| Lube blend | RMI - MAGNESIUM STEARATE NF | 4.000 | 1.250 kg |
| Layer | | 400.000 | |
| Weight: | | | |
| Totals(wt) | | 800.00 | 250.000 kg |
| Totals (tablets) | | 1 | 312,500 |

Procedure

Sustained Release Layer (Layer 1)

1. Set aside 0.5 kg of Purified Water to be used in step 4.

2. Prepare a solution using the scale up amounts of Povidone K-30, and the remaining Purified Water from step 1.

3. Blend the pre-blend scale up amounts of Phenylephrine HCl USP, Calcium Phosphate Dibasic Dihydrate, and Prosolv SMCC 90 with a high shear mixer/granulator for 10 minutes.

4. With the mixer/granulator on, pump the solution prepared in step 2 in to the mixer/granulator. After completion, stop the mixer/granulator and rinse the container with the Purified water set aside in step 1. Pump the rinse water to the mixer/granulator with mixer on. Turn off mixer when completed.

5. Charge the pre-blend scale up amount of Methocel K4M premium to the mixer/granulator and mix for 1 minute.

6. Dry the granulation until the LOD is 4% or less.

7. Pass the dried granulation through a Fitzpatrick Fitzmill fitted with a 14 mesh screen.

8. Screen the final blend scale up amount of Methocel K4M premium through a 14 mesh screen.

9. Blend the milled granulation from step 7 and the screened materials from step 8 using a V-blender for 20 minutes.

10. Screen the lube scale up amount of Magnesium Stearate using a 30 mesh screen.

11. Transfer the screened Magnesium Stearate to the V-blender and blend for 3 minutes. When completed discharge into drums double lined with polyethylene bags.

Immediate Release Layer (Layer 2)

1. Set aside 0.5 kg of Purified Water to be used in step 4.

2. Prepare a solution using the scale up amounts Povidone K-30, and the remaining Purified Water from step 1.

3. Blend the pre-blend scale up amounts of Carbetapentane Citrate, Prosolv SMCC 90, Lactose Monohydrate #316, Sodium Starch Glycolate, and FD&C Blue #1 Aluminum Lake using a high shear mixer/granulator for 10 minutes.

4. With the mixer/granulator on, pump the solution prepared in step 2 in to the mixer/granulator. After completion, stop the mixer/granulator and rinse the container with the Purified Water set aside in step 1. Pump the rinse water to the mixer/granulator with mixer on. Allow mixture to mix for an additional minute then turn mixer/granulator off.

5. Dry the granulation until the LOD is 4% or less.

6. Pass the dried granulation through a Fitzpatrick Fitzmill fitted with a 14 mesh screen.

7. Screen the final blend scale up amounts of Prosolv SMCC 90, Lactose Monohydrate #316, Sodium Starch Glycolate, and FD&C Blue #1 Aluminum Lake through a 14 mesh screen.

8. Blend the milled granulation from step 6 and the screened materials from step 7 using a V-blender for 20 minutes.

9. Screen the lube scale up amount of Magnesium Stearate using a 30 mesh screen.

10. Transfer the screened Magnesium Stearate to the V-blender and blend for 3 minutes. When completed discharge into proper drums double lined with polyethylene bags.

Compress bi-layered tablets using a rotary bi-layer tablet press where in each tablet layer 1 is 400.0 mg and layer 2 is 400 mg.

The above examples 41-62 illustrate how to manufacture a bi-layered tablet containing a decongestant, i.e., phenylephrine hydrochloride, in (at least) a first layer and an antihistamine and/or a decongestant and/or an expectorant in (at least) a second layer. Non-limiting examples of other possible active ingredients in an exemplary range as described in the above Table 1 can be employed depending on the specific therapeutic effect desired.

### EXAMPLE 63

### Bi-Layered Tablet (Wet Granulation)

A bi-layered tablet in accordance with the present invention which contains phenylephrine hydrochloride in both an immediate release layer and a sustained release layer and carbetapentane citrate in both an immediate release layer and a sustained release layer is illustrated as follows:

| | | |
|---|---|---|
| | Weight/tablet | Weight batch |
| Ingredients | (mg) | (Kg) |

Layer 1 (Immediate release)

| | | |
|---|---|---|
| Carbetapentane citrate | 15.0 | 22.5 |
| Phenylepherine hydrochloride | 15.0 | 22.5 |
| Silicified Microcrystalline Cellulose | 74.2 | 111.3 |
| Croscarmellose Sodium | 10.0 | 15.0 |
| Magnesium Stearate | 0.8 | 1.2 |

Layer 2 (Sustained release)

| | | |
|---|---|---|
| Carbetapentane citrate | 45.0 | 67.5 |
| Phenylepherine hydrochloride | 45.0 | 67.5 |
| Microcrystalline Cellulose (PH 102) | 20.0 | 30.0 |
| Povidone | 8.0 | 12.0 |
| Methocel K4M Premium | 150.0 | 225.0 |
| Magnesium Stearate | 2.0 | 3.0 |
| Total | 385.0 | 577.5 |

Procedure:

(a) Immediate release layer #1: Mix the prescreened (# 30 mesh) phenylephrine hydrochloride,carbetapentane citrate, silicified microcrystalline cellulose and croscarmellose sodium, in a V shaped blender for 20 minutes. Add prescreened (# 40 mesh) magnesium stearate in a V shaped blender and mix for 3 minutes.

(b) Sustained release layer #2: Mix the phenylephrine hydrochloride, carbetapentane citrate, Methocel K4M Premium and microcrystalline cellulose in a high shear mixer/granulator for 10 minutes. Granulate the above blend using a 30% povidone solution (12.0 Kg povidone in 28.0 Kg purified water). Dry the granulation until the LOD is less than 2.0%. Screen granules through a US sieve size # 14. Add the granules and the prescreened magnesium stearate in a V shaped blender and mix for 3 minutes.

Manufacture bi-layered tablets using a rotary bi-layer tablet press where in each tablet layer #1 is 115 mg and layer #2 is 270 mg.

### EXAMPLE 64

### Bi-Layered Tablet (Wet granulation)

A bi-layered tablet in accordance with the present invention which comprises phenylephrine hydrochloride and guaifenesin in two different sustained release layers is illustrated as follows:

Formula

| | | Amounts | |
|---|---|---|---|
| Process Steps | Ingredients | Dose(mg) | Scale-Up |
| Wet Mix | RMI - PURIFIED WATER | 55.000 | 17.188 kg |
| Wet Mix | RMI - POVIDONE K-30 USP | 15.000 | 4.688 kg |
| Pre-blend | RMA - GUAIFENESIN USP | 600.000 | 187.500 kg |
| Pre-blend | RMA - PHENYLEPHRINE HCL USP | 40.000 | 12.500 kg |
| Pre-blend | RMI - CAL PHOSPHATE DIBASIC | 40.000 | 12.500 kg |
| | ANHYD | | |
| Pre-blend | RMI - METHOCEL K4M PREMIUM USP | 16.000 | 5.000 kg |
| Final blend | RMI - STEARIC ACID NF | 4.500 | 1.406 kg |
| Final blend | RMI - METHOCEL K4M PREMIUM USP | 79.700 | 24.906 kg |
| Lube blend | RMI - MAGNESIUM STEARATE NF | 4.500 | 1.406 kg |
| Color blend | DYE - FD&C BLUE #1 ALM LAKE | 0.300 | 0.094 kg |
| Total (wt) | | 800.000 | 250.000 kg |
| Total (tablets) | | 1 | 312,500 |

Procedure

1. Set aside 0.5 kg of purified water to be used as a rinse in step 4.

2. Prepare a solution using the scale up amounts of Povidone K-30, and the remaining Purified Water.

3. Blend the pre-blend scale up amounts of Guaifenesin USP, Phenylephrine HCl USP, and Calcium Phosphate Dibasic Anhydrous using a high sheer mixer/granulator for 10 minutes.

4. With the mixer/granulator on, pump the solution prepared in step 2 in to the mixer/granulator. After completion, stop the mixer/granulator and rinse the container with the 0.5 kg of Purified water set aside in step 1. Pump the rinse water to the mixer/granulator with mixer on. Turn off mixer when completed.

5. Charge the pre-blend scale up amount of Methocel K4M premium to the mixer/granulator and mix for 1 minute.

6. Dry the granulation until the LOD is 1% or less.

7. Pass the dried granulation through a Fitzpatrick Fitzmill fitted with a 109 screen.

8. Screen the final blend raw materials through a 12 mesh screen.

9. Blend the milled granulation from step 7 and the screened materials from step 8 using a V-blender for 20 minutes.

10. Screen the lube blend material through a 30 mesh screen.

11. Charge the screened material from step 10 to the V-blender and blend for 2 minutes.

12. Discharge half of the blended product into proper drums double lined with polyethylene bags and set aside for step 16.

13. Screen the color blend material through a 30 mesh screen.

14. Charge the screened color blend material to the V-blender and blend for 1 minute.

15. Discharge into proper drums double lined with polyethylene bags and set aside for step 16.

16. Compress bi-layered tablets using a rotary bi-layer tablet press where in each tablet layer 1 is 400.0 mg's and layer 2 is 400.0 mg's.

### EXAMPLE 65

### Extended Release Suspension (Gel)

An extended release suspension (in the form of a gel) in accordance with the present invention which contains a phenylephrine hydrochloride ion-exchange complex and promethazine hydrochloride is illustrated as follows (the phenylephrine hydrochloride is used in a controlled release form since it has a shorter half-life than the promethazine hydrochloride):

| Ingredients | Amount/5 ml | |
|---|---|---|
| Phenylepherine hydrochloride | Equivalent to 30 mg of | |
| Ion-Exchange Complex | Phenylepherine | |
| Promethazine HCl | 25 | mg |
| Eudragit ® L 100 | 0.2 to 2.8 | g |
| Glycerin | 315 | mg |
| Polysorbate 80 | 1.5 | mg |
| Carbomer (e.g., Carbopol ® 974) | 37.5 | mg |
| Methyl Paraben | 9 | mg |
| Propyl Paraben | 1 | mg |
| Saccharin Sodium cryst., USP | 0.1 | mg |
| Artificial Grape Flavor | 5 | mg |
| FD&C Red # 40 Dye | 0.5 | mg |
| Sodium Hydroxide | to pH of 6.5 | |
| Water | to 5 | ml |

Procedure:

(1) Add the appropriate amount of sodium polystyrene sulphonate USP (e.g. Amberlite® IRP 69) to a phenylephrine hydrochloride solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eudragit® L 100, Kollidon® MAE, Aquacoat®V cPD) and dry the granules.

(5) Mill the granules, if needed.

(6) To an appropriate amount of water add the following ingredients and dissolve: promethazine hydrochloride, Carbomer (e.g., Carbopol® 974), glycerin, polysorbate 80, methyl paraben, propyl paraben, artificial grape flavor, FD&C red # 40 dye.

(7) Add milled granules.

(8) Add water to 80% of final volume.

(9) Agitate at suitable rate to avoid settling of the suspension and maintain a homogeneous product mixture.

(10) Neutralize the solution to a pH of 6.5 (range 5.5 to 7.5) to form a gel using a 1N sodium hydroxide solution. QS with water to make final volume of 5 ml per dose.

(11) Fill in suitable containers.

### EXAMPLE 66

### Extended Release Suspension (Liquid)

An extended release suspension (in the form of a liquid) in accordance with the present invention which contains a phenylephrine hydrochloride ion-exchange complex and promethazine hydrochloride is illustrated as follows:

| Ingredients | Amount/5 ml | |
|---|---|---|
| Phenylepherine hydrochloride | Equivalent to 45 mg of | |
| Ion-Exchange Complex | Phenylepherine hydrochloride | |
| Promethazine HCl | 25 | mg |
| Eudragit ® L 100 | 0.2 to 2.8 | g |
| Silica, colloidal anhydrous, NF | 100 | mg |
| Glycerin | 740 | mg |
| Xylitol, NF | 800 | mg |
| Sodium Citrate, USP | 100 | mg |
| Saccharin Sodium cryst., USP, | 0.1 | mg |
| Sodium Benzoate | 7.5 | mg |
| Citric Acid Monohydrate, USP | 8.0 | mg |
| Artificial Grape Flavor | 5 | mg |
| FD&C Red # 40 Dye | 0.5 | mg |
| Water | q.s to 5 | ml |

Manufacturing Process for 1000 L Batch:

Add the appropriate amount of sodium polystyrene sulphonate USP (e.g. Amberlite® IRP 69) to a phenylephrine hydrochloride solution. Stir the mix for 12 hrs to allow complete drug/resin complex formation. Separate and dry the insoluble drug/resin complex. Granulate the drug/resin complex with a delayed release/enteric polymer (e.g. Eudragit® L 100, Kollidon® MAE, Aquacoat® CPD) and dry the granules. Mill the granules, if needed.

In a suitably sized stainless steel vessel, dissolve saccharin sodium, sodium benzoate, citric acid, and sodium citrate in approximately 50 L of warm (about 45° C.), purified water. In another large stainless steel drum mix the silica, phenylephrine hydrochloride ion-exchange complex, and promethazine hydrochloride until a uniform and consistent mixture is obtained. In a separate 1000 L stainless steel tank equipped with a suitably sized homogenizer/disperser add about 100 L of purified water. With the homogenizer on, add the silica mixture containing phenylephrine hydrochloride ion-exchange complex, and promethazine hydrochloride. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients and homogenize for 15 minutes.

### EXAMPLE 67

### Extended Release Suspension (Liquid)

An extended release suspension (in the form of a liquid) in accordance with the present invention which contains a codeine phosphate ion-exchange complex, phenylephrine tannate and chlorpheniramine tannate is illustrated as follows:

| Ingredients | Amount/5 ml | |
|---|---|---|
| Codeine phosphate | Equivalent to 45 mg | |
| Ion-exchange complex | Codeine phosphate | |
| Phenylepherine Tannate | 75.0 | |
| Chlorpheniramine Tannate | 4.5 | |
| Eudragit ® L 100 | 0.2 to 2.8 | grams |
| Silica, colloidal anhydrous, NF | 100 | mg |
| Glycerin | 740 | mg |
| Xylitol, NF | 800 | mg |
| Sodium Citrate, USP | 100 | mg |
| Saccharin Sodium cryst., USP, | 0.1 | mg |
| Sodium Benzoate | 7.5 | mg |
| Citric Acid Monohydrate, USP | 8.0 | mg |
| Artificial Grape Flavor | 5 | mg |
| FD&C Red # 40 Dye | 0.5 | mg |
| Water | q.s to 5 | ml |

Manufacturing Process for 1000 Kg Batch:

Add the appropriate amount of sodium polystyrene sulphonate USP (e.g. Amberlite® IRP 69) to a codeine phosphate solution. Stir the mix for 12 hrs to allow complete drug/resin complex formation. Separate and dry the insoluble drug/resin complex. Granulate the drug/resin complex with a delayed release/enteric polymer (e.g. Eudragit® L 100, Kollidon® MAE, Aquacoat® CPD) and dry the granules. Mill the granules, if needed.

In a suitably sized stainless steel vessel, dissolve saccharin sodium, sodium benzoate, citric acid, and sodium citrate in approximately 50 L of warm (about 45° C.), purified water. In another large stainless steel drum mix the silica and the codeine phosphate ion-exchange complex, phenylephrine tannate, and the chlorpheniramine tannate until a uniform and consistent mixture is obtained. In a separate 1000 L stainless steel tank equipped with a suitably sized homogenizer/disperser add about 100 L of purified water. With the homogenizer on, add the silica mixture containing the codeine phosphate ion-exchange complex, the phenylephrine tannate, and the chlorpheniramine tannate. Add the previously prepared solution of saccharin sodium, sodium benzoate, citric acid, and sodium citrate to the 1000 L tank. Rinse the first vessel with about 2 L of water and transfer the rinse to the 1000 L tank. Add the remaining ingredients and homogenize for 15 minutes.

### EXAMPLE 68

### Extended Release Suspension

An extended release suspension which contains a hydrocodone bitartrate ion-exchange complex, a dexchlorpheniramine maleate ion-exchange complex and a phenylephrine hydrochloride ion-exchange complex is illustrated as follows:

| Ingredients | Amount/5 ml | |
|---|---|---|
| Hydrocodonee Bitartrate | Equivalent to 8 mg of | |
| Ion-Exchange Complex | Hydrocodonee bitartarate | |
| Dexchlorpheniramine Maleate | Equivalent to 6 mg of | |
| Ion-Exchange Complex | Dexchlorpheniramine Maleate | |
| Phenylepherine HCl | Equivalent to 10 mg of | |
| Ion-Exchange Complex | Phenylepherine HCl | |
| Eudragit ® L 100 | 0.2 to 2.8 | g |
| Glycerin | 315 | mg |
| Polysorbate 80 | 1.5 | mg |
| Carbomer (e.g., Carbopol ® 974) | 15 | mg |
| Methyl Paraben | 9 | mg |
| Propyl Paraben | 1 | mg |
| Artificial Grape Flavor | 5 | mg |
| FD&C Red # 40 Dye | 0.5 | mg |

| | |
|---|---|
| Water | q.s |

The formula described above serves as a non-limiting example. Any active drug which is in the form of a salt, such as an antihistamine, codeine, or dihydrocodeine, can be incorporated as an ion-exchange resin complex.

Procedure:

(1) Add the appropriate amount of sodium polystyrene sulphonate USP (e.g. Amberlite® IRP 69) to a dexchlorpheniramine maleate, hydrocodone bitartrate and phenylephrine HCl solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eugragit® L 100, Kollidon® MAE, Aquacoa®t cPD) and dry the granules.

(5) Mill the granules, if needed.

(6) To an appropriate amount of water add the following ingredients and dissolve: Carbomer (e.g., Carbopol® 974), glycerin, polysorbate 80, methyl paraben, propyl paraben, artificial grape flavor, FD&C red # 40 dye.

(7) Add milled granules.

(8) Add water to make up to a final volume.

(9) Agitate at suitable rate to avoid settling of the suspension and maintain a homogeneous product mixture.

(10) Fill in suitable containers ensuring that the product is homogeneous throughout the filling operation.

### EXAMPLE 69

### Suspension Formula

A suspension formula which comprises codeine phosphate and phenylephrine tannate is illustrated as follows:

| Ingredients | g/100 mL | kg/batch |
|---|---|---|
| Codeine Phosphate | 0.20 | 1.667 |
| Phenylepherine Tannate | 0.80 | 6.667 |
| Silica, colloidal anhydrous, NF | 1.73 | 14.417 |
| Hydroxyethylcellulose, NF | 0.05 | 0.417 |
| Sorbitol Solution 70% (non-crystallizing), NF | 34.00 | 283.333 |
| Glycerol | 14.75 | 122.917 |
| Xylitol, NF | 16.00 | 133.333 |
| Sodium Citrate, USP | 2.00 | 16.667 |
| Saccharin Sodium cryst., USP, | 0.01 | 0.083 |
| Sodium Benzoate, NF | 0.15 | 1.250 |
| Citric Acid Monohydrate, USP | 0.16 | 1.333 |
| Strawberry Flavor | 0.15 | 1.250 |
| Banana Flavor | 0.15 | 1.250 |
| Purified Water | 49.55 | 412.917 |
| Total Amount | 120.00 | 1000.000 |

Manufacturing Process for 1000 kg Batch:

In a suitably sized stainless steel vessel, dissolve saccharin sodium, sodium benzoate, citric acid, and sodium citrate in approximately 50 L of warm (about 45° C.), purified water. In another large stainless steel drum mix the silica, codeine phosphate and micronized phenylephrine tannate until a uniform and consistent mixture is obtained. In a separate 1000 L stainless steel tank equipped with a suitably sized homogenizer/disperser add about 100 L of purified water. With the homogenizer on, add the silica mixture containing phenylephrine tannate and codeine phosphate Add the previously prepared solution of saccharin sodium, sodium benzoate, citric acid, and sodium citrate to the 1000 L tank. Rinse the first vessel with about 2 L of water and transfer the rinse to the 1000 L tank. Add the remaining ingredients and homogenize for 15 minutes. Filter product through a 10 micron filter and fill in appropriately sized containers.

To make products with other agents such as antihistamines, decongestants, or expectorants, one or more combinations of each of the ingredients in a range as described in Table 1 below can be made depending on the specific therapeutic effect desired.

### EXAMPLE 70

### Liquid Formula

A liquid dosage form which comprises phenylephrine hydrochloride is illustrated as follows:

| Ingredients | Per 5 mL | | Per 425 L | |
|---|---|---|---|---|
| Codeine Phosphate USP | 30 | mg | 2.550 | kg |
| Phenylepherine Hydrochloride USP | 10.0 | mg | 0.850 | kg |
| Methyl Paraben USP | 9.0 | mg | 0.765 | kg |
| Propyl Paraben USP | 1.0 | mg | 0.085 | kg |
| Propylene Glycol USP | 259 | mg | 22.016 | kg |
| Saccharin Sodium USP | 3.18 | mg | 0.270 | kg |
| Citric Acid USP | 5.0 | mg | 0.425 | kg |
| Strawberry Flavor | 10 | mg | 0.850 | kg |
| Banana Flavor | 10 | mg | 0.850 | kg |
| Sorbitol Solution 70% USP | 3212.5 | mg | 273.1 | kg |
| Purified Water, as required to q.s. to | 5.0 | mL | 425 | L |

Manufacturing Process for 425 L Batch Size:

In a suitably sized stainless steel vessel, dissolve methyl paraben and propyl paraben in approximately 50 L of warm (about 45° C.), purified water. Add about half of the propylene glycol and mix for about 1 hr. In a separate 1000 L stainless steel tank equipped with a suitably sized agitator, add about 50 L of purified water. With the agitator on, add phenylephrine hydrochloride, codeine phosphate, saccharin sodium and citric acid and dissolve. Add the previously prepared paraben/propylene glycol solution to the 1000 L tank. Rinse the first vessel with about 2 L of water and transfer the rinsate to the 1000 L tank. Add the remaining propylene glycol to a suitably sized stainless steel vessel and dissolve the strawberry and banana flavors. Transfer this to the 1000 L tank. Rinse the container with 2 L of purified water and transfer to the 1000 L tank. With the agitator on, add the sorbitol solution 70% to the 1000 L tank. In a suitably sized stainless steel vessel, dissolve the codeine phosphate in about 5 L of purified water and transfer to the 1000 L tank. Rinse the container with about 2 L of purified water and transfer to the 1000 L tank. Stop the agitator and let the solution stand for 15 minutes. QS to 425 L with purified water. Filter product through a 1 micron filter and fill in appropriately sized containers.

To make products with other antihistamines, decongestants, or expectorants, one or more combinations of each of the ingredients in a range as described in Table 1 above can be made depending on the specific therapeutic effect desired.

### EXAMPLE 71

### Liquid Formula

A liquid dosage form in accordance with the present invention which comprises diphenhydramine hydrochloride, dihydrocodeine bitartrate and phenylephrine hydrochloride is illustrated as follows:

| Ingredients | Per 5 mL | | Per 425 L | |
|---|---|---|---|---|
| Diphenhydramine Hydrochloride USP | 12.5 | mg | 1.063 | kg |
| Dihydrocodeine Bitartrate USP | 10.0 | mg | 0.850 | kg |
| Phenylepherine Hydrochloride USP | 10.0 | mg | 0.850 | kg |
| Methyl Paraben USP | 9.0 | mg | 0.765 | kg |
| Propyl Paraben USP | 1.0 | mg | 0.085 | kg |
| Propylene Glycol USP | 259 | mg | 22.016 | kg |
| Saccharin Sodium USP | 3.18 | mg | 0.270 | kg |
| Citric Acid USP | 5.0 | mg | 0.425 | kg |
| Strawberry Flavor | 10 | mg | 0.850 | kg |
| Banana Flavor | 10 | mg | 0.850 | kg |
| Sorbitol Solution 70% USP | 3212.5 | mg | 273.1 | kg |
| Purified Water, as required to q.s. to | 5.0 | mL | 425 | L |

Manufacturing process for 425 L batch size: In a suitably sized stainless steel vessel, dissolve methyl paraben and propyl paraben in approximately 50 L of warm (about 45° C.), purified water. Add about half of the propylene glycol and mix for about 1 hr. In a separate 1000 L stainless steel tank equipped with a suitably sized agitator, add about 50 L of purified water. With the agitator on, add phenylephrine hydrochloride, diphenhydramine hydrochloride, saccharin sodium and citric acid and dissolve. Add the previously prepared paraben/propylene glycol solution to the 1000 L tank. Rinse the first vessel with about 2 L of water and transfer the rinsate to the 1000 L tank. Add the remaining propylene glycol to a suitably sized stainless steel vessel and dissolve the strawberry and banana flavors. Transfer this to the 1000 L tank. Rinse the container with 2 L of purified water and transfer to the 1000 L tank. With the agitator on, add the sorbitol solution 70% to the 1000 L tank. In a suitably sized stainless steel vessel, dissolve the dihydrocodeine bitartrate in about 5 L of purified water and transfer to the 1000 L tank. Rinse the container with about 2 L of purified water and transfer to the 1000 L tank. Stop the agitator and let the solution stand for 15 minutes. Adjust pH to 3.5 to 5.5 with either HCl or NaOH, qs to 425 L with purified water. Filter product through a 1 micron filter and fill in appropriately sized containers.

To make products with other analgesics, decongestants, antitussives or expectorants, one or more combinations of each of the ingredients in a range as described in Table 1 below can be made depending on the specific therapeutic effect desired.

### EXAMPLE 72

### Suspension Formula

A suspension formula in accordance with the present invention which comprises diphenhydramine hydrochloride and phenylephrine tannate is illustrated as follows:

| Ingredients | 100 mL | | 833.33 L | |
|---|---|---|---|---|
| Diphenhydramine Hydrochloride | 0.250 | | 2.083 | |
| Phenylepherine Tannate | 0.800 | | 6.667 | |
| Silica, colloidal anhydrous, NF | 1.73 | | 14.417 | |
| Hydroxyethylcellulose, NF | 0.05 | | 0.417 | |
| Sorbitol Solution 70% (non-crystallizing), | 34.00 | | 283.333 | |
| NF | | | | |
| Glycerol | 15.00 | | 125.000 | |
| Xylitol, NF | 16.00 | | 133.333 | |
| Sodium Citrate, USP | 2.00 | | 16.667 | |
| Saccharin Sodium cryst., USP, | 0.01 | | 0.083 | |
| Sodium Benzoate, NF | 0.15 | | 1.250 | |
| Citric Acid Monohydrate, USP | 0.16 | | 1.333 | |
| Strawberry Flavor | 0.15 | | 1.250 | |
| Banana Flavor | 0.15 | | 1.250 | |
| Purified Water | QS | | QS | |
| Total Amount | 100 | mL | 833.33 | L |

Manufacturing process for 833.33 L batch: In a suitably sized stainless steel vessel, dissolve saccharin sodium, sodium benzoate, citric acid, and sodium citrate in approximately 50 L of warm (about 45° C.), purified water. In another large stainless steel drum mix the silica, diphenhydramine hydrochloride and micronized phenylephrine tannate until a uniform and consistent mixture is obtained. In a separate 1000 L stainless steel tank equipped with a suitably sized homogenizer/disperser add about 100 L of purified water. With the homogenizer on, add the silica mixture containing phenylephrine tannate and diphenhydramine hydrochloride. Add the previously prepared solution of saccharin sodium, sodium benzoate, citric acid, and sodium citrate to the 1000 L tank. Rinse the first vessel with about 2 L of water and transfer the rinsate to the 1000 L tank. Add the remaining ingredients, qs to 833.33 L and homogenize for 15 minutes. Filter product through a 10 micron filter and fill in appropriately sized containers.

To make products with other antihistamines, decongestants, antitussives, or expectorants, one or more combinations of each of the ingredients in a range as described in Table 1 below can be made depending on the specific therapeutic effect desired.

### EXAMPLE 73

### Bi-Layered Tablet (Direct Compression)

A bi-layered tablet in accordance with the present invention which comprises diphenhydramine hydrochloride in one layer and phenylephrine hydrochloride and diphenhydramine hydrochloride in the other layer is illustrated as follows:

| | | |
|---|---|---|
| | Weight/tablet | Weight/1 kg batch |
| Ingredients | (mg) | (in grams) |

Layer 1 (Immediate release)

| | | |
|---|---|---|
| Diphenhydramine Hydrochloride | 100.00 | 137.9 |
| Silicified Microcrystalline | 114.0 | 157.2 |
| Cellulose | | |
| Sodium Starch Glycolate | 10.0 | 13.8 |
| Magnesium Stearate | 1.0 | 1.38 |

Layer 2 (Sustained release)

| | | |
|---|---|---|
| Phenylepherine HCl | 20.0 | 27.6 |
| Diphenhydramine Hydrochloride | 100.0 | 137.9 |
| Lactose Monohydrate | 50.0 | 68.97 |
| Dicalcium Phosphate | 50.0 | 68.97 |
| Kollidon SR | 260.0 | 358.6 |
| Stearic acid | 15.0 | 20.7 |
| Magnesium Stearate | 5.0 | 6.9 |
| Total | 725.00 | 1000.0 |

Manufacturing Process

(a) Immediate release layer: Screen all ingredients through a USP sieve size # 30. Blend diphenhydramine hydrochloride, silicified microcrystalline cellulose and sodium starch glycolate in a twin shell blender for 20 minutes. Add magnesium stearate which acts as a lubricant, to the above blend and mix for 3 minutes.

(b) Sustained release layer: Screen all ingredients through a USP sieve size # 30. Preblend a portion (about ⅓) of the Kollidon SR and all the diphenydramine hydrochloride for 15 minutes. Add the remaining Kollidon SR, phenylephrine hydrochloride, lactose monohydrate and dicalcium phosphate to the above preblend and mix for an additional 20 minutes. Add stearic acid and magnesium stearate to the above blend and mix for three minutes.

Manufacture bi-layered tablets using a rotary bi-layer tablet press where in each tablet the immediate release layer is 225 mg and the sustained release layer is 500 mg.

By using the process described above, a bi-layered tablet of the following composition may be manufactured through direct compression:

| | |
|---|---|
| | Weight/tablet |
| Ingredients | (mgs) |

Layer 1 (Immediate Release)

| | |
|---|---|
| Diphenhydramine Hydrochloride | 50 |
| Silicified Microcrystalline Cellulose | 133.5 |
| Sodium Starch Glycholate | 15 |
| Magnesium Stearate | 1.5 |

Layer 2 (Sustained Release)

| | |
|---|---|
| Phenylepherine HCl | 20 |
| Diphenhydramine Hydrochloride | 50 |
| Dicalcium Phosphate | 100 |
| Kollidon SR | 260 |
| Stearic Acid | 15 |
| Magnesium Stearate | 5 |
| Total | 650 |

### EXAMPLE 74

### Bi-Layered Tablet (Wet Granulation)

A bi-layered tablet in accordance with the present invention which comprises diphenhydramine hydrochloride in one layer and pseudoephedrine hydrochloride and diphenhydramine hydrochloride in the other layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Diphenhydramine Hydrochloride | 100.0 | 100.0 |
| Silicified Microcrystalline Cellulose | 111.0 | 111.0 |
| Povidone | 3.0 | 3.0 |
| Croscarmellose Sodium | 10.0 | 10.0 |
| Magnesium Stearate | 1.0 | 1.0 |

Layer 2 (Sustained release)

| | | |
|---|---|---|
| Pseudoephedrine HCl | 120.0 | 120.0 |
| Diphenhydramine Hydrochloride | 100.0 | 100.0 |
| Microcrystalline Cellulose (PH 102) | 105.0 | 105.0 |
| Lactose Monohydrate | 100.00 | 100.00 |
| Dicalcium Phosphate | 100.0 | 100.0 |
| Povidone | 15.0 | 15.0 |
| Methocel K4M Premium | 10.0 | 210.0 |
| Stearic Acid | 20.0 | 20.0 |
| Magnesium Stearate | 5.0 | 5.0 |
| Total | 800.0 | 800.0 |

Manufacturing Process

(a) Immediate release layer: Screen all ingredients through a USP sieve size # 30. Blend diphenhydramine hydrochloride, silicified microcrystalline cellulose and croscarmellose sodium in a high shear mixer/granulator for 10 minutes. Granulate the above blend using a 30% povidone solution (4.3 gms povidone in 14.3 gms purified water). Dry the granulation until the loss on drying (LOD) is less than 2.0%. Screen the dried granulation through a USP sieve size # 14. Add the screened granulation and the prescreened magnesium stearate to the above blend and mix for 3 minutes.

(b) Sustained release layer: Screen all ingredients through a USP sieve size # 30. Blend the pseudoephedrine hydrochloride, diphenhydramine hydrochloride, Prosolv® (silicified microcrystalline cellulose), lactose monohydrate, dicalcium phosphate, Methocel K4M Premium and stearic acid in a high shear mixer/granulator for 10 minutes. Granulate the above blend using a 30% povidone solution. Dry the granulation until the LOD is less than 2.0%. Screen granules through a USP sieve size # 14. Add granules and the prescreened magnesium stearate to the above blend and mix for 3 minutes.

Manufacture bi-layered tablets using a rotary bi-layer tablet press where in each tablet the immediate release layer is 225 mgs and the sustained release layer is 575 mgs.

By using the process described above, a bi-layered tablet of the following composition may be manufactured through wet granulation:

| | |
|---|---|
| | Weight/tablet |
| Ingredients | (mgs) |

Layer 1 (Immediate Release)

| | |
|---|---|
| Diphenhydramine Hydrochloride | 100 |
| Silicified Microcrystalline cellulose | 129.5 |
| Povidone | 4 |
| Croscarmellose sodium | 15 |
| Magnesium Stearate | 1.5 |

Layer 2 (Sustained Release)

| | |
|---|---|
| Pseudoephedrine HCl | 120 |
| Diphenhydramine Hydrochloride | 100 |
| Microcrystalline Cellulose 102 | 30 |
| Lactose Monohydrate | 100 |
| Dicalcium Phosphate | 100 |
| Povidone | 15 |
| Hydroxypropylmethylcellulose | 210 |
| Stearic Acid | 20 |
| Magnesium Stearate | 5 |
| Total | 950 |

The above examples 63-74 illustrate how to manufacture a bi-layered tablet containing an antihistamine, i.e., diphenhydramine hydrochloride in one layer and a decongestant and/or an antitussive and/or an expectorant and/or an analgesic in the other layer (optionally in combination with diphenhydramine hydrochloride). One or more of the non-limiting examples of active ingredients which may be used in combination with diphenhydramine hydrochloride depending on the specific therapeutic effect desired are listed in the above Table 1 together with exemplary ranges thereof.

### EXAMPLE 75

### Extended Release Suspension

| Ingredients | Amount/5 ml |
|---|---|
| Diphenhydramine hydrochloride | Equivalent to 12.5 mg |
| ion-exchange complex | Diphenhydramine hydrochloride |
| Phenylepherine ion-exchange complex | Equivalent to 10 mg |
| | Phenylepherine HCl |
| Eudragit ® L 100 | 0.2 to 2.8 grams |
| Glycerin | 315 mg |
| Polysorbate 80 | 1.5 mg |
| Carbomer (e.g., Carbopol ® 974) | 15 mg |
| Methyl Paraben | 9 mg |
| Propyl Paraben | 1 mg |
| Artificial grape flavor | 5 mg |
| FD&C red # 40 dye | 0.5 mg |
| Water | q.s. to 5 mL |

The formula described above serves as a non-limiting example. Any active drug which is in the form of a salt, such as diphenhydramine hydrochloride, codeine phosphate, pseudoephedrine hydrochloride, morphine sulfate, or meperidine hydrochloride can be incorporated as an ion-exchange resin complex.

Procedure:

(1) Add the appropriate amount of sodium polystyrene sulphonate USP (e.g. Amberlite® IRP 88N) to a diphenhydramine hydrochloride and phenylephrine HCl solution.

(2) Stir the mix for 12 hrs to allow complete drug/resin complex formation.

(3) Separate and dry the insoluble drug/resin complex.

(4) Granulate the drug/resin complex with a delayed release/enteric polymer (e.g. Eugragit® L 100, Kollidon® MAE, Aquacoat® PD) and dry the granules.

(5) Mill the granules, if needed.

(6) To an appropriate amount of water add the following ingredients and dissolve: Carbomer (e.g., Carbopol® 974), glycerin, polysorbate 80, methyl paraben, propyl paraben, artificial grape flavor, FD&C red # 40 dye.

(7) Add milled granules.

(8) Add water to make up to a final volume.

(9) Adjust pH to the desired viscosity to keep all the solid materials in suspension.

(10) Agitate at suitable rate to avoid settling of the suspension and maintain a homogeneous product mixture.

(11) Fill in suitable containers ensuring that the product is homogeneous throughout the filling operation.

### EXAMPLE 76

### Bi-Layered Tablet (Wet Granulation)

A bilayered tablet in accordance with the present invention which comprises diphenhydramine hydrochloride and guaifenesin in both layers as sustained release layers is illustrated as follows.

Formula

| | | Amounts | | | |
|---|---|---|---|---|---|
| Process Steps | Ingredients | Dose(mg) | | Scale-Up | |
| Wet Mix | PURIFIED WATER | 55.000 | | 13.750 | kg |
| Wet Mix | POVIDONE K-30 USP | 15.000 | | 3.750 | kg |
| Pre-blend | GUAIFENESIN USP | 600.000 | | 150.000 | kg |
| Pre-blend | DIPHENHYDRAMINE HCL USP | 100.000 | | 25.000 | kg |
| Pre-blend | CAL PHOSPHATE DIBASIC ANHYD | 40.000 | | 10.000 | kg |
| Pre-blend | METHOCEL K4M PREMIUM USP | 16.000 | | 4.000 | kg |
| Final blend | STEARIC ACID NF | 4.500 | | 1.130 | kg |
| Final blend | METHOCEL K4M PREMIUM USP | 89.700 | | 22.430 | kg |
| Lube blend | MAGNESIUM STEARATE NF | 4.500 | | 1.130 | kg |
| Color blend | FD&C BLUE #1 ALM LAKE | 0.300 | | 0.075 | kg |
| | Total (wt) | 925.000 | g | 231.3 | kg |
| | Total | 1 | | 250,000 | |
| | (tablets) | | | | |

Procedure

12. Set aside 0.5 kg of purified water to be used as a rinse in step 4.

13. Prepare a solution using the scale up amounts of Povidone K-30, and the remaining Purified Water.

14. Blend the pre-blend scale up amounts of Guaifenesin USP, Diphenhydramine HCl USP, and Calcium Phosphate Dibasic Anhydrous using a high shear mixer/granulator for 10 minutes.

15. With the mixer/granulator on, pump the solution prepared in step 2 in to the mixer/granulator. After completion, stop the mixer/granulator and rinse the container with the 0.5 kg of purified water set aside in step 1. Pump the rinse water to the mixer/granulator with mixer on. Turn off mixer when completed.

16. Charge the pre-blend scale up amount of Methocel K4M premium to the mixer/granulator and mix for 1 minute.

17. Dry the granulation until the LOD is 1% or less.

18. Pass the dried granulation through a Fitzpatrick Fitzmill fitted with a 109 screen.

19. Screen the final blend raw materials through a 12 mesh screen.

20. Blend the milled granulation from step 7 and the screened materials from step 8 using a V-blender for 20 minutes.

21. Screen the lube blend material through a 30 mesh screen.

22. Charge the screened material from step 10 to the V-blender and blend for 2 minutes.

23. Discharge half of the blended product into properly labeled drums double lined with polyethylene bags and set aside for step 16.

24. Screen the color blend material through a 30 mesh screen.

25. Charge the screened color blend material to the V-blender and blend for 1 minute.

26. Discharge into properly labeled drums double lined with polyethylene bags and set aside for step 16.

27. Compress bi-layered tablets using a rotary bi-layer tablet press where in each tablet layer 1 is 450 mg and layer 2 is 475 mg.

### EXAMPLE 77

### Bi-Layered Tablet (Wet Granulation)

A bilayered tablet in accordance with the present invention which comprises diphenhydramine hydrochloride in a sustained release layer and carbetapentane in an immediate release layer is illustrated as follows.

Formula

| | | Amounts | |
|---|---|---|---|
| Process Steps | Ingredients | Dose(mg) | Scale-Up |

Sustained Release Layer

| | | | | | |
|---|---|---|---|---|---|
| Wet Mix | PURIFIED WATER | | 48.000 | 15.000 | kg |
| Wet Mix | POVIDONE K-30 USP | | 12.000 | 3.750 | kg |
| Pre-blend | DIPHENHYDRAMINE HCl USP | | 50.000 | 15.630 | kg |
| Pre-blend | CAL PHOSPHATE DIBASIC | | 15.000 | 4.690 | kg |
| | DIHYD | | | | |
| Pre-blend | PROSOLV ® SMCC 90 | | 125.000 | 39.063 | kg |
| Pre-blend | METHOCEL K4M PREMIUM USP | | 50.000 | 15.625 | kg |
| Final blend | METHOCEL K4M PREMIUM USP | | 144.000 | 45.000 | kg |
| Lube blend | MAGNESIUM STEARATE NF | | 4.000 | 1.250 | kg |
| | | Layer Weight | 400.00 | | |

Immediate Release Layer

| | | | | | |
|---|---|---|---|---|---|
| Wet Mix | PURIFIED WATER | | 24.000 | 7.500 | kg |
| Wet Mix | POVIDONE K-30 USP | | 6.000 | 1.875 | kg |
| Pre-blend | CARBETAPENTANE CITRATE | | 40.000 | 12.500 | kg |
| Pre-blend | PROSOLV SMCC 90 | | 100.000 | 31.250 | kg |
| Pre-blend | LACTOSE MONOHYDRATE #316 | | 40.000 | 12.500 | kg |
| Pre-blend | SODIUM STARCH GLYCOLATE | | 8.000 | 2.500 | kg |
| Pre-blend | FD&C BLUE #1 ALUMINUM | | 0.156 | 0.048 | kg |
| | LAKE | | | | |
| Final blend | PROSOLV SMCC 90 | | 145.688 | 45.528 | kg |
| Final blend | LACTOSE MONOHYDRATE #316 | | 40.000 | 12.500 | kg |
| Final blend | SODIUM STARCH GLYCOLATE | | 16.000 | 5.000 | kg |
| Final blend | FD&C BLUE #1 ALUMINUM | | 0.156 | 0.048 | kg |
| | LAKE | | | | |
| Lube blend | MAGNESIUM STEARATE NF | | 4.000 | 1.250 | kg |
| | | Layer Weight | 400.000 | | |
| | | Totals(wt) | 800.000 | 250.000 | kg |

| | | |
|---|---|---|
| Totals | 1 | 312,500 |
| (tablets) | | |

Procedure

Sustained Release Layer (Layer 1)

1. Set aside 0.5 kg of purified water to be used in step 4.

2. Prepare a solution using the scale up amounts of Povidone K-30, and the remaining Purified Water from step 1.

3. Blend the pre-blend scale up amounts of Diphenhydramine HCl USP, Calcium Phosphate Dibasic Dihydrate, and Prosolv SMCC 90 with a high shear mixer/granulator for 10 minutes.

4. With the mixer/granulator on, pump the solution prepared in step 2 in to the mixer/granulator. After completion, stop the mixer/granulator and rinse the container with the purified water set aside in step 1. Pump the rinse water to the mixer/granulator with mixer on. Turn off mixer when completed.

5. Charge the pre-blend scale up amount of Methocel K4M premium to the mixer/granulator and mix for 1 minute.

6. Dry the granulation until the LOD is 4% or less.

7. Pass the dried granulation through a Fitzpatrick Fitzmill fitted with a 14 mesh screen.

8. Screen the final blend scale up amount of Methocel K4M premium through a 14 mesh screen.

9. Blend the milled granulation from step 7 and the screened materials from step 8 using a V-blender for 20 minutes.

10. Screen the lube scale up amount of Magnesium Stearate using a 30 mesh screen.

11. Transfer the screened Magnesium Stearate to the V-blender and blend for 3 minutes. When completed discharge into properly identified drums double lined with polyethylene bags.

Immediate Release Layer (Layer 2)

1. Set aside 0.5 kg of purified water to be used in step 4.

2. Prepare a solution using the scale up amount of Povidone K-30, and the remaining purified water from step 1.

3. Blend the pre-blend scale up amounts of Carbetapentane Citrate, Prosolv SMCC 90, Lactose Monohydrate #316, Sodium Starch Glycolate, and FD&C Blue #1 Aluminum Lake using a high shear mixer/granulator for 10 minutes.

4. With the mixer/granulator on, pump the solution prepared in step 2 in to the mixer/granulator. After completion, stop the mixer/granulator and rinse the container with the purified water set aside in step 1. Pump the rinse water to the mixer/granulator with mixer on. Allow mixture to mix for an additional minute then turn mixer/granulator off.

5. Dry the granulation until the LOD is 4% or less.

6. Pass the dried granulation through a Fitzpatrick Fitzmill fitted with a 14 mesh screen.

7. Screen the final blend scale up amounts of Prosolv SMCC 90, Lactose Monohydrate #316, Sodium Starch Glycolate, and FD&C Blue #1 Aluminum Lake through a 14 mesh screen.

8. Blend the milled granulation from step 6 and the screened materials from step 7 using a V-blender for 20 minutes.

9. Screen the lube scale up amount of Magnesium Stearate using a 30 mesh screen.

10. Transfer the screened Magnesium Stearate to the V-blender and blend for 3 minutes. When completed discharge into properly identified drums double lined with polyethylene bags.

11. Compress bi-layered tablets using a rotary bi-layer tablet press where in each tablet layer 1 is 400.0 mg and layer 2 is 400 mg.

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. A pharmaceutical dosage form comprising (a) a first drug which is selected from decongestants, antitussives, expectorants, analgesics and antihistamines, and (b) a second drug which is selected from decongestants, antitussives, expectorants, analgesics and antihistamines, wherein the dosage form provides a plasma concentration within a therapeutic range of the second drug over a period which is coextensive with at least about 70% of a period over which the dosage form provides a plasma concentration within a therapeutic range of the first drug.

2. The dosage form of claim 1, wherein the first drug comprises a decongestant.

3. The dosage form of claim 2, wherein the decongestant comprises at least one of phenylephrine, pseudoephedrine and pharmaceutically acceptable salts thereof.

4. The dosage form of claim 3, wherein the decongestant comprises at least one of phenylephrine and a pharmaceutically acceptable salt thereof.

5. The dosage form of claim 3, wherein the decongestant comprises at least one of pseudoephedrine and a pharmaceutically acceptable salt thereof.

6. The dosage form of claim 1, wherein the first drug comprises an antitussive.

7. The dosage form of claim 6, wherein the antitussive comprises at least one of a morphine derivative, dextromethorphan, carbetapentane, chlophedianol, benzonatate, caramiphen, noscapine and pharmaceutically acceptable salts thereof.

8. The dosage form of claim 7, wherein the antitussive comprises at least one of codeine, dihydrocodeine, hydrocodone and pharmaceutically acceptable salts thereof.

9. The dosage form of claim 7, wherein the antitussive comprises at least one of carbetapentane and a pharmaceutically acceptable salt thereof.

10. The dosage form of claim 7, wherein the antitussive comprises at least one of chlophedianol and a pharmaceutically acceptable salt thereof.

11. The dosage form of claim 1, wherein the first drug comprises an antihistamine.

12. The dosage form of claim 11, wherein the antihistamine comprises at least one of astemizole, azatadine, azelastine, bromodiphenhydramine, brompheniramine, carbinoxamine, cetirizine, chlorcyclizine, clemastine, chlorothen, chlorpheniramine, cyclizine, cyproheptadine, desloratadine, dexbrompheniramine, dexbrompheniramine, dimethindene, diphenhydramine, diphenylpyraline, doxylamine, fexofenadine, hydroxyzine, isothipendyl, loratadine, methapyrilene, montelukast, phenindamine, pheniramine, phenyltoloxamine, promethazine, prophenpyridamine, pyrilamine, terfenadine, thenyldiamine, thonzylamine, trimeprazine, tripelennamine, triprolidine, and pharmaceutically acceptable salts thereof.

13. The dosage form of claim 1 1, wherein the antihistamine comprises at least one of promethazine, diphenhydramine, chlorpheniramine and pharmaceutically acceptable salts thereof.

14. The dosage form of claim 11, wherein the antihistamine comprises at least one of carbinoxamine and a pharmaceutically acceptable salt thereof.

15. The dosage form of claim 1, wherein the first drug comprises an analgesic.

16. The dosage form of claim 15, wherein the analgesic comprises at least one of aspirin, acetaminophen, ibuprofen, ketoprofen, naproxen, sodium naproxen, meloxicam, hydrocodone, oxycodone, morphine, meperidine, and fentanyl.

17. The dosage form of claim 1, wherein the first drug comprises an expectorant.

18. The dosage form of claim 17, wherein the expectorant comprises at least one of guaifenesin and a pharmaceutically acceptable salt thereof.

19. The dosage form of claim 2, wherein the second drug comprises an antitussive.

20. The dosage form of claim 19, wherein the antitussive comprises at least one of a morphine derivative, dextromethorphan, carbetapentane, chlophedianol, benzonatate, caramiphen, noscapine and pharmaceutically acceptable salts thereof.

21. The dosage form of claim 19, wherein the antitussive comprises at least one of codeine, dihydrocodeine, hydrocodone and pharmaceutically acceptable salts thereof.

22. The dosage form of claim 2, wherein the second drug comprises an antihistamine.

23. The dosage form of claim 22, wherein the antihistamine comprises at least one of astemizole, azatadine, azelastine, bromodiphenhydramine, brompheniramine, carbinoxamine, cetirizine, chlorcyclizine, clemastine, chlorothen, chlorpheniramine, cyclizine, cyproheptadine, desloratadine, dexbrompheniramine, dexbrompheniramine, dimethindene, diphenhydramine, diphenylpyraline, doxylamine, fexofenadine, hydroxyzine, isothipendyl, loratadine, methapyrilene, montelukast, phenindamine, pheniramine, phenyltoloxamine, promethazine, prophenpyridamine, pyrilamine, terfenadine, thenyldiamine, thonzylamine, trimeprazine, tripelennamine, triprolidine, and pharmaceutically acceptable salts thereof.

24. The dosage form of claim 22, wherein the antihistamine comprises at least one of promethazine, diphenhydramine, chlorpheniramine and pharmaceutically acceptable salts thereof.

25. The dosage form of claim 22, wherein the antihistamine comprises at least one of carbinoxamine and a pharmaceutically acceptable salt thereof.

26. The dosage form of claim 2, wherein the second drug comprises an analgesic.

27. The dosage form of claim 26, wherein the analgesic comprises at least one of aspirin, acetaminophen, ibuprofen, ketoprofen, naproxen, sodium naproxen, meloxicam, hydrocodone, oxycodone, morphine, meperidine, and fentanyl.

28. The dosage form of claim 2, wherein the second drug comprises an expectorant.

29. The dosage form of claim 28, wherein the expectorant comprises at least one of guaifenesin and a pharmaceutically acceptable salt thereof.

30. The dosage form of claim 7, wherein the second drug comprises a decongestant.

31. The dosage form of claim 30, wherein the decongestant comprises at least one of phenylephrine, pseudoephedrine and pharmaceutically acceptable salts thereof.

32. The dosage form of claim 6, wherein the second drug comprises an antihistamine.

33. The dosage form of claim 32, wherein the antihistamine comprises at least one of astemizole, azatadine, azelastine, bromodiphenhydramine, brompheniramine, carbinoxamine, cetirizine, chlorcyclizine, clemastine, chlorothen, chlorpheniramine, cyclizine, cyproheptadine, desloratadine, dexbrompheniramine, dexbrompheniramine, dimethindene, diphenhydramine, diphenylpyraline, doxylamine, fexofenadine, hydroxyzine, isothipendyl, loratadine, methapyrilene, montelukast, phenindamine, pheniramine, phenyltoloxamine, promethazine, prophenpyridamine, pyrilamine, terfenadine, thenyldiamine, thonzylamine, trimeprazine, tripelennamine, triprolidine, and pharmaceutically acceptable salts thereof.

34. The dosage form of claim 32, wherein the antihistamine comprises at least one of promethazine, diphenhydramine, chlorpheniramine and pharmaceutically acceptable salts thereof.

35. The dosage form of claim 32, wherein the antihistamine comprises at least one of carbinoxamine and a pharmaceutically acceptable salt thereof.

36. The dosage form of claim 6, wherein the second drug comprises an analgesic.

37. The dosage form of claim 36, wherein the analgesic comprises at least one of aspirin, acetaminophen, ibuprofen, ketoprofen, naproxen, sodium naproxen, meloxicam, hydrocodone, oxycodone, morphine, meperidine, and fentanyl.

38. The dosage form of claim 6, wherein the second drug comprises an expectorant.

39. The dosage form of claim 38, wherein the expectorant comprises at least one of guaifenesin and a pharmaceutically acceptable salt thereof.

40. The dosage form of claim 12, wherein the second drug comprises a decongestant.

41. The dosage form of claim 40, wherein the decongestant comprises at least one of phenylephrine, pseudoephedrine and pharmaceutically acceptable salts thereof.

42. The dosage form of claim 12, wherein the second drug comprises an antitussive.

43. The dosage form of claim 42, wherein the antitussive comprises at least one of a morphine derivative, dextromethorphan, carbetapentane, chlophedianol, benzonatate, caramiphen, noscapine and pharmaceutically acceptable salts thereof.

44. The dosage form of claim 42, wherein the antitussive comprises at least one of codeine, dihydrocodeine, hydrocodone and pharmaceutically acceptable salts thereof.

45. The dosage form of claim 11, wherein the second drug comprises an analgesic.

46. The dosage form of claim 45, wherein the analgesic comprises at least one of aspirin, acetaminophen, ibuprofen, ketoprofen, naproxen, sodium naproxen, meloxicam, hydrocodone, oxycodone, morphine, meperidine, and fentanyl.

47. The dosage form of claim 11, wherein the second drug comprises an expectorant.

48. The dosage form of claim 47, wherein the expectorant comprises at least one of guaifenesin and a pharmaceutically acceptable salt thereof.

49. The dosage form of claim 16, wherein the second drug comprises a decongestant.

50. The dosage form of claim 49, wherein the decongestant comprises at least one of phenylephrine, pseudoephedrine and pharmaceutically acceptable salts thereof.

51. The dosage form of claim 16, wherein the second drug comprises an antitussive.

52. The dosage form of claim 51, wherein the antitussive comprises at least one of a morphine derivative, dextromethorphan, carbetapentane, chlophedianol, benzonatate, caramiphen, noscapine and pharmaceutically acceptable salts thereof.

53. The dosage form of claim 51, wherein the antitussive comprises at least one of codeine, dihydrocodeine, hydrocodone and pharmaceutically acceptable salts thereof.

54. The dosage form of claim 16, wherein the second drug comprises an antihistamine.

55. The dosage form of claim 54, wherein the antihistamine comprises at least one of astemizole, azatadine, azelastine, bromodiphenhydramine, brompheniramine, carbinoxamine, cetirizine, chlorcyclizine, clemastine, chlorothen, chlorpheniramine, cyclizine, cyproheptadine, desloratadine, dexbrompheniramine, dexchlorpheniramine, dimethindene, diphenhydramine, diphenylpyraline, doxylamine, fexofenadine, hydroxyzine, isothipendyl, loratadine, methapyrilene, montelukast, phenindamine, pheniramine, phenyltoloxamine, promethazine, prophenpyridamine, pyrilamine, terfenadine, thenyldiamine, thonzylamine, trimeprazine, tripelennamine, triprolidine, and pharmaceutically acceptable salts thereof.

56. The dosage form of claim 54, wherein the antihistamine comprises at least one of promethazine, diphenhydramine, chlorpheniramine and pharmaceutically acceptable salts thereof.

57. The dosage form of claim 54, wherein the antihistamine comprises promethazine.

58. The dosage form of claim 15, wherein the second drug comprises at least one of carbinoxamine and a pharmaceutically acceptable salt thereof.

59. The dosage form of claim 58, wherein the expectorant comprises at least one of guaifenesin and a pharmaceutically acceptable salt thereof.

60. The dosage form of claim 1, wherein a plasma half-life of the second drug differs from a plasma half-life of the first drug by at least about 2 hours.

61. The dosage form of claim 1, wherein a plasma half-life of the second drug differs from a plasma half-life of the first drug by at least about 3 hours.

62. The dosage form of claim 1, wherein a plasma half-life of the second drug differs from a plasma half-life of the first drug by at least about 4 hours.

63. The dosage form of claim 61, wherein the period of a plasma concentration within the therapeutic range of the second drug is coextensive with at least about 80% of the period of a plasma concentration within the therapeutic range of the first drug.

64. The dosage form of claim 60, wherein the period of a plasma concentration within the therapeutic range of the second drug is coextensive with at least about 90% of the period of a plasma concentration within the therapeutic range of the first drug.

65. The dosage form of claim 1, wherein the period of a plasma concentration within the therapeutic range of the second drug is coextensive with at least about 95% of the period of a plasma concentration within the therapeutic range of the first drug.

66. The dosage form of claim 1, wherein the period of a plasma concentration within the therapeutic range of the second drug is substantially coextensive with the period of a plasma concentration within the therapeutic range of the first drug.

67. The dosage form of claim 1, wherein the dosage form comprises a tablet.

68. The dosage form of claim 67, wherein the tablet comprises a matrix which comprises one of the first drug and the second drug and has dispersed therein particles which comprise the other one of the first drug and the second drug.

69. The dosage form of claim 1, wherein the dosage form comprises one of a solution and a suspension.

70. A bi-layered tablet which comprises a first layer and a second layer, the first layer comprising a first drug which is selected from decongestants, antitussives, expectorants, analgesics and antihistamines, and the second layer comprising a second drug which is selected from decongestants, antitussives, expectorants, analgesics and antihistamines and is different from the first drug, wherein the bi-layered tablet provides a plasma concentration within a therapeutic range of the second drug over a period which is coextensive with at least about 70% of a period over which the bi-layered tablet provides a plasma concentration within a therapeutic range of the first drug.

71. The bi-layered tablet of claim 70, wherein the first drug is selected from one or more of phenylephrine, pseudoephedrine, codeine, dihydrocodeine, hydrocodone, dextromethorphan, carbetapentane, chlophedianol, benzonatate, caramiphen, noscapine, guaifenesin, astemizole, azatadine, azelastine, bromodiphenhydramine, brompheniramine, carbinoxamine, cetirizine, chlorcyclizine, clemastine, chlorothen, chlorpheniramine, cyclizine, cyproheptadine, desloratadine, dexbrompheniramine, dexchlorpheniramine, dimethindene, diphenhydramine, diphenylpyraline, doxylamine, fexofenadine, hydroxyzine, isothipendyl, loratadine, methapyrilene, montelukast, phenindamine, pheniramine, phenyltoloxamine, promethazine, prophenpyridamine, pyrilamine, terfenadine, thenyldiamine, thonzylamine, trimeprazine, tripelennamine, triprolidine, and pharmaceutically acceptable salts thereof, aspirin, acetaminophen, ibuprofen, ketoprofen, naproxen, sodium naproxen, meloxicam, hydrocodone, oxycodone, morphine, meperidine, and fentanyl.

72. The bi-layered tablet of claim 71, wherein the second drug is different from the first drug and is selected from one or more of phenylephrine, pseudoephedrine, codeine, dihydrocodeine, hydrocodone, dextromethorphan, carbetapentane, chlophedianol, benzonatate, caramiphen, noscapine, guaifenesin, astemizole, azatadine, azelastine, bromodiphenhydramine, brompheniramine, carbinoxamine, cetirizine, chlorcyclizine, clemastine, chlorothen, chlorpheniramine, cyclizine, cyproheptadine, desloratadine, dexbrompheniramine, dexchlorpheniramine, dimethindene, diphenhydramine, diphenylpyraline, doxylamine, fexofenadine, hydroxyzine, isothipendyl, loratadine, methapyrilene, montelukast, phenindamine, pheniramine, phenyltoloxamine, promethazine, prophenpyridamine, pyrilamine, terfenadine, thenyldiamine, thonzylamine, trimeprazine, tripelennamine, triprolidine, and pharmaceutically acceptable salts thereof, aspirin, acetaminophen, ibuprofen, ketoprofen, naproxen, sodium naproxen, meloxicam, hydrocodone, oxycodone, morphine, meperidine, and fentanyl.

73. The bi-layered tablet of claim 71, wherein the period of a plasma concentration within the therapeutic range of the second drug is coextensive with at least about 80% of the period of a plasma concentration within the therapeutic range of the first drug.

74. The bi-layered tablet of claim 72, wherein the period of a plasma concentration within the therapeutic range of the second drug is coextensive with at least about 90% of the period of a plasma concentration within the therapeutic range of the first drug.

75. The bi-layered tablet of claim 74, wherein the period of a plasma concentration within the therapeutic range of the second drug is coextensive with at least about 95% of the period of a plasma concentration within the therapeutic range of the first drug.

76. The bi-layered tablet of claim 70, wherein the period of a plasma concentration within the therapeutic range of the second drug is substantially coextensive with the period of a plasma concentration within the therapeutic range of the first drug.

77. The bi-layered tablet of claim 76, wherein a plasma half-life of the second drug differs from a plasma half-life of the first drug by at least about 2 hours.

78. The bi-layered tablet of claim 74, wherein a plasma half-life of the second drug differs from a plasma half-life of the first drug by at least about 3 hours.

79. The bi-layered tablet of claim 70, wherein one of the first and second layers is an immediate release layer.

80. The bi-layered tablet of claim 70, wherein the first and second layers are controlled release layers.

81. A multi-layered tablet which comprises at least a first layer and a second layer, wherein the first layer comprises a first drug which is selected from decongestants, antitussives, expectorants, analgesics and antihistamines and the second layer is a controlled release layer and comprises a second drug which is selected from decongestants, antitussives, expectorants, analgesics and antihistamines and is different from the first drug.

82. The multi-layered tablet of claim 81, wherein the first layer is an immediate release layer.

83. The multi-layered tablet of claim 82, wherein the second layer is a controlled release layer.

84. The multi-layered tablet of claim 82, wherein the second drug has a plasma half-life which differs from the plasma half-life of the first drug by at least about 3 hours.

85. The multi-layered tablet of claim 84, wherein the tablet provides a plasma concentration within a therapeutic range of the second drug over a period which is coextensive with at least about 90% of a period over which the tablet provides a plasma concentration within a therapeutic range of the first drug.

86. The multi-layered tablet of claim 81, wherein the layers are discrete zones which are arranged adjacent to each other.

87. The multi-layered tablet of claim 81, wherein one of the first and second layers is partially or completely surrounded by the other one of the first and second layers.

88. A pharmaceutical dosage form which comprises (a) a first drug which is selected from antihistamines and has a first plasma half-life and (b) a second drug which is selected from decongestants, antitussives, analgesics and expectorants and has a second plasma half-life that differs from the first plasma half-life by at least about 3 hours, wherein the dosage form provides a plasma concentration within a therapeutic range of the second drug over a period which is coextensive with at least about 80% of a period over which the dosage form provides a plasma concentration within a therapeutic range of the first drug.

89. The dosage form of claim 88, wherein the first plasma half-life is longer by at least about 4 hours than the second plasma half-life.

90. The dosage form of claim 88, wherein the first plasma half-life is longer by at least about 6 hours than the second plasma half-life.

91. The dosage form of claim 88, wherein the first plasma half-life is at least about 8 hours.

92. The dosage form of claim 89, wherein the period of a plasma concentration within the therapeutic range of the at least one second drug is coextensive with at least about 90% of the period of a plasma concentration within the therapeutic range of the first drug.

93. The dosage form of claim 89, wherein the period of a plasma concentration within the therapeutic range of the at least one second drug is coextensive with at least about 95% of the period of a plasma concentration within the therapeutic range of the first drug.

94. The dosage form of claim 88, wherein the period of a plasma concentration within the therapeutic range of the at least one second drug is substantially coextensive with the period of a plasma concentration within the therapeutic range of the first drug.

95. The dosage form of claim 88, wherein the dosage form is associated with instructions to administer the dosage form three or fewer times per day.

96. The dosage form of claim 88, wherein the dosage form is associated with instructions to administer the dosage form once or twice per day.

97. The dosage form of claim 88, wherein the dosage form comprises a tablet.

98. The dosage form of claim 97, wherein the second drug comprises a decongestant.

99. The dosage form of claim 98, wherein the antihistamine is selected from astemizole, azatadine, azelastine, bromodiphenhydramine, brompheniramine, carbinoxamine, cetirizine, chlorcyclizine, clemastine, chlorothen, chlorpheniramine, cyclizine, cyproheptadine, desloratadine, dexbrompheniramine, dexchlorpheniramine, dimethindene, diphenhydramine, diphenylpyraline, doxylamine, fexofenadine, hydroxyzine, isothipendyl, loratadine, methapyrilene, montelukast, phenindamine, pheniramine, phenyltoloxamine, promethazine, prophenpyridamine, pyrilamine, terfenadine, thenyldiamine, thonzylamine, trimeprazine, tripelennamine, triprolidine, and pharmaceutically acceptable salts thereof.

100. The dosage form of claim 98, wherein the antihistamine comprises at least one of promethazine, diphenhydramine, chlorpheniramine and pharmaceutically acceptable salts thereof.

101. The dosage form of claim 98, wherein the antihistamine comprises carbinoxamine.

102. The dosage form of claim 99, wherein the second drug comprises a decongestant.

103. The dosage form of claim 102, wherein the decongestant comprises at least one of phenylephrine, pseudoephedrine and pharmaceutically acceptable salts thereof.

104. The dosage form of claim 99, wherein the second drug comprises an antitussive.

105. The dosage form of claim 104, wherein the antitussive comprises at least one of a morphine derivative, dextromethorphan, carbetapentane, chlophedianol, benzonatate, caramiphen, noscapine and pharmaceutically acceptable salts thereof.

106. The dosage form of claim 104, wherein the antitussive comprises at least one of codeine, dihydrocodeine, hydrocodone and pharmaceutically acceptable salts thereof.

107. The dosage form of claim 99, wherein the second drug comprises an analgesic.

108. The dosage form of claim 107, wherein the analgesic comprises at least one of aspirin, acetaminophen, ibuprofen, ketoprofen, naproxen, sodium naproxen, meloxicam, hydrocodone, oxycodone, morphine, meperidine, and fentanyl.

109. The dosage form of claim 99, wherein the second drug comprises an expectorant.

110. The dosage form of claim 109, wherein the expectorant comprises at least one of guaifenesin and a pharmaceutically acceptable salt thereof.

111. A method of alleviating a condition which can be alleviated by administration of an antihistamine and at least one other condition which can be alleviated by administration of a drug which is at least one of a decongestant, antitussive, expectorant, and analgesic, wherein the method comprises administering the dosage form of claim 88 to a subject in need thereof.

112. A process of making the pharmaceutical dosage form of claim 1, wherein the method comprises preparing a first composition which comprises the first drug and a second composition which comprises the second drug, and combining the first and the second compositions.

113. The process of claim 112, wherein the first and second compositions are combined by using a tablet press.

114. A pharmaceutical dosage form comprising (a) a first drug which is selected from decongestants, antitussives, expectorants and analgesics, and (b) a second drug which is selected from decongestants, antitussives, expectorants and analgesics and is different from the first drug, wherein the dosage form provides a plasma concentration within a therapeutic range of the second drug over a period which is coextensive with at least about 70% of a period over which the dosage form provides a plasma concentration within a therapeutic range of the first drug and wherein the dosage form is substantially free of antihistamines.

115. The dosage form of claim 114, wherein the first drug comprises a decongestant.

116. The dosage form of claim 115, wherein the decongestant comprises at least one of phenylephrine, pseudoephedrine and pharmaceutically acceptable salts thereof.

117. The dosage form of claim 114, wherein the first drug comprises an antitussive.

118. The dosage form of claim 117, wherein the antitussive comprises at least one of a morphine derivative, dextromethorphan, carbetapentane, chlophedianol, benzonatate, caramiphen, noscapine and pharmaceutically acceptable salts thereof.

119. The dosage form of claim 117, wherein the antitussive comprises at least one of codeine, dihydrocodeine, hydrocodone and pharmaceutically acceptable salts thereof.

120. The dosage form of claim 117, wherein the antitussive comprises at least one of carbetapentane and a pharmaceutically acceptable salt thereof.

121. The dosage form of claim 114, wherein the first drug comprises an analgesic.

122. The dosage form of claim 121, wherein the analgesic comprises at least one of aspirin, acetaminophen, ibuprofen, ketoprofen, naproxen, sodium naproxen, meloxicam, hydrocodone, oxycodone, morphine, meperidine, and fentanyl.

123. The dosage form of claim 121, wherein the analgesic comprises a NSAID.

124. The dosage form of claim 114, wherein the first drug comprises an expectorant.

125. The dosage form of claim 124, wherein the expectorant comprises at least one of guaifenesin and a pharmaceutically acceptable salt thereof.

126. The dosage form of claim 115, wherein the second drug comprises an antitussive.

127. The dosage form of claim 126, wherein the antitussive comprises at least one of a morphine derivative, dextromethorphan, carbetapentane, chlophedianol, benzonatate, caramiphen, noscapine and pharmaceutically acceptable salts thereof.

128. The dosage form of claim 127, wherein the antitussive comprises at least one of codeine, dihydrocodeine, hydrocodone and pharmaceutically acceptable salts thereof.

129. The dosage form of claim 115, wherein the second drug comprises an analgesic.

130. The dosage form of claim 129, wherein the analgesic comprises at least one of aspirin, acetaminophen, ibuprofen, ketoprofen, naproxen, sodium naproxen, meloxicam, hydrocodone, oxycodone, morphine, meperidine, and fentanyl.

131. The dosage form of claim 129, wherein the analgesic comprises a NSAID.

132. The dosage form of claim 115, wherein the second drug comprises an expectorant.

133. The dosage form of claim 132, wherein the expectorant comprises at least one of guaifenesin and a pharmaceutically acceptable salt thereof.

134. The dosage form of claim 117, wherein the second drug comprises an analgesic.

135. The dosage form of claim 134, wherein the analgesic comprises at least one of aspirin, acetaminophen, ibuprofen, ketoprofen, naproxen, sodium naproxen, meloxicam, hydrocodone, oxycodone, morphine, meperidine, and fentanyl.

136. The dosage form of claim 134, wherein the analgesic comprises a NSAID.

137. The dosage form of claim 117, wherein the second drug comprises an expectorant.

138. The dosage form of claim 137, wherein the expectorant comprises at least one of guaifenesin and a pharmaceutically acceptable salt thereof.

139. The dosage form of claim 121, wherein the second drug comprises an expectorant.

140. The dosage form of claim 139, wherein the expectorant comprises at least one of guaifenesin and a pharmaceutically acceptable salt thereof.

141. The dosage form of claim 114, wherein a plasma half-life of the second drug differs from a plasma half-life of the first drug by at least about 2 hours.

142. The dosage form of claim 141, wherein the period of a plasma concentration within the therapeutic range of the second drug is substantially coextensive with the period of a plasma concentration within the therapeutic range of the first drug.

143. The dosage form of claim 114, wherein the dosage form comprises a tablet.

144. A bi-layered tablet which comprises a first layer and a second layer, the first layer comprising a first drug which is selected from decongestants, antitussives, expectorants and analgesics, and the second layer comprising a second drug which is different from the first drug and is selected from decongestants, antitussives, expectorants and analgesics, wherein the bi-layered tablet provides a plasma concentration within a therapeutic range of the second drug over a period which is coextensive with at least about 70% of a period over which the bi-layered tablet provides a plasma concentration within a therapeutic range of the first drug and wherein the bi-layered tablet is substantially free of antihistamines.

145. The bi-layered tablet of claim 144, wherein the first drug is selected from one or more of phenylephrine, pseudoephedrine, codeine, dihydrocodeine, hydrocodone, dextromethorphan, carbetapentane, chlophedianol, benzonatate, caramiphen, noscapine, guaifenesin, and pharmaceutically acceptable salts thereof, aspirin, acetaminophen, ibuprofen, ketoprofen, naproxen, sodium naproxen, meloxicam, hydrocodone, oxycodone, morphine, meperidine, and fentanyl.

146. The bi-layered tablet of claim 145, wherein the second drug is different from the first drug and is selected from one or more of phenylephrine, pseudoephedrine, codeine, dihydrocodeine, hydrocodone, dextromethorphan, carbetapentane, chlophedianol, benzonatate, caramiphen, noscapine, guaifenesin, and pharmaceutically acceptable salts thereof, aspirin, acetaminophen, ibuprofen, ketoprofen, naproxen, sodium naproxen, meloxicam, hydrocodone, oxycodone, morphine, meperidine, and fentanyl.

147. The bi-layered tablet of claim 144, wherein the period of a plasma concentration within the therapeutic range of the second drug is substantially coextensive with the period of a plasma concentration within the therapeutic range of the first drug.

148. The bi-layered tablet of claim 147, wherein a plasma half-life of the second drug differs from a plasma half-life of the first drug by at least about 2 hours.

149. The bi-layered tablet of claim 144, wherein one of the first and second layers is an immediate release layer.

150. The bi-layered tablet of claim 144, wherein the first and second layers are controlled release layers.

151. A method of alleviating a condition which can be alleviated by administration of at least two drugs which are selected form decongestants, antitussives, expectorants and analgesics, wherein the method comprises administering the dosage form of claim 114 to a subject in need thereof.

152. The dosage form of claim 1, wherein the dosage form comprises at least one of carbinoxamine and a pharmaceutically acceptable salt thereof as a first drug and at least one of pseudoephedrine and a pharmaceutically acceptable salt thereof as a second drug.

153. The bi-layered tablet of claim 70, wherein the dosage form comprises at least one of carbinoxamine and a pharmaceutically acceptable salt thereof as a first drug and at least one of pseudoephedrine and a pharmaceutically acceptable salt thereof as a second drug.

154. The dosage form of claim 114, wherein the dosage form comprises an NSAID as a first drug and at least one of pseudoephedrine, phenylephrine, guaifenesin, codeine, dihydrocodeine, hydrocodone, carbetapentane and pharmaceutically acceptable salts thereof as a second drug.

155. The dosage form of claim 154, wherein the NSAID comprises at least one of ibuprofen, ketoprofen, naproxen and sodium naproxen.

156. The bi-layered tablet of claim 144, wherein the dosage form comprises an NSAID as a first drug and at least one of pseudoephedrine, phenylephrine, guaifenesin, codeine, dihydrocodeine, hydrocodone, carbetapentane and pharmaceutically acceptable salts thereof as a second drug.
